SEQUENCE LISTING (3 sequences)

SEQ ID NO: 1 - protein, 414 aa, Homo sapiens
MSEYIRVTEDENDEPIEIPSEDDGTVLLSTVTAQFPGACGLRYRNPVSQCMRGVRLVEGILHAPDAGWGNLVYVVNYPKDNKRKMDETDASSAVKVKRAVQKTSDLIVLGLPWKTTEQDLKEYFSTFGEVLMVQVKKDLKTGHSKGFGFVRFTEYETQVKVMSQRHMIDGRWCDCKLPNSKQSQDEPLRSRKVFVGRCTEDMTEDELREFFSQYGDVMDVFIPKPFRAFAFVTFADDQIAQSLCGEDLIIKGISVHISNAEPKHNSNRQLERSGRFGGNPGGFGNQGGFGNSRGGGAGLGNNQGSNMGGGMNFGAFSINPAMMAAAQAALQSSWGMMGMLASQQNQSGPSGNNQNQGNMQREPNQAFGSGNNSYSGSNSGAAIGWGSASNAGSGSGFNGGFGSSMDSKSSGWGM

SEQ ID NO: 2 - DNA, 20 bp, Artificial Sequence
  synthetic construct; forward primer
tttggagcgctgcgaagttg

SEQ ID NO: 3 - DNA, 18 bp, Artificial Sequence
  synthetic construct; reverse primer
agctgtggttccgtgttg

(19) United States
(12) Patent Application Publication
(10) Pub. No.: US 2015/0143553 A1
(43) Pub. Date: May 21, 2015

(54) Methods And Compositions Comprising A Drosophila Model Of Amyotrophic Lateral Sclerosis

(71) Applicant: The Arizona Board of Regents on Behalf of The University of Arizona, Tucson, AZ (US)

(72) Inventors: Daniela Zarnescu, Tucson, AZ (US); Patricia S. Estes, Tucson, AZ (US)

(21) Appl. No.: 14/508,610

(22) Filed: Oct. 7, 2014

Related U.S. Application Data
(60) Provisional application No. 61/905,048, filed on Nov. 15, 2013.

(51) Int. Cl. A61K 49/00; A01K 67/033

(52) U.S. Cl. CPC A61K 49/0008; A01K 67/0339; A01K 2267/0331; A01K 2207/15
USPC 800/3; 800/9; 536/28.7

(57) ABSTRACT

In an aspect, the invention relates to a method of screening for a therapeutic for amyotrophic lateral sclerosis. In an aspect, the invention relates to transgenic Drosophila. In an aspect, the invention relates to a Drosophila model of amyotrophic lateral sclerosis. This abstract is intended as a scanning tool for purposes of searching in the particular art and is not intended to be limiting of the present invention.

### CROSS-REFERENCE TO RELATED APPLICATIONS

This application claims benefit of U.S. Provisional Application No. 61/905,048, filed Nov. 15, 2013. Application No. 61/905,048, filed Nov. 15, 2013, is hereby incorporated herein by reference in its entirety.

### REFERENCE TO SEQUENCE LISTING

The Sequence Listing submitted Oct. 7, 2014 as a text file named “37435_{—}0001U2_Sequence_Listing.txt,” created on Oct. 7, 2014, and having a size of 4,276 bytes is hereby incorporated by reference pursuant to 37 C.F.R. §1.52(e)(5).

### BACKGROUND

Amyotrophic Lateral Sclerosis (ALS) is an adult onset, progressive neurological disorder characterized by selective degeneration and death of motor neurons in the motor cortex and the spinal cord. Approximately 10% of all ALS cases are inherited and have been linked to a number of genes including superoxide dismutase (SOD1) and more recently C9ORF72. However, 90% of the known ALS cases are sporadic and remain poorly understood. The ALS pathology includes ubiquitin positive cytoplasmic bodies, which have been shown to contain a 28 kDa fragment corresponding to the C-terminus domain of TDP-43 protein together with the full length TDP-43. Several missense mutations have been identified in TDP-43, the majority of which lie within the C-terminal region, indicating that this domain can be involved in the pathogenesis of ALS. Pathways and compounds with neuroprotective potential for TDP-43-associated phenotypes can be determined, which can impact a wide spectrum of ALS cases.

Despite advances in understanding the physiology and pathophysiology of amyotrophic lateral sclerosis, there is still a scarcity of compounds that are potent, efficacious, and safe in the treatment or amelioration of amyotrophic lateral sclerosis. These needs and other needs are satisfied by the present invention.

### SUMMARY

Disclosed herein is an in vivo method of screening for a therapeutic for amyotrophic lateral sclerosis, the method comprising administering a candidate therapeutic for amyotrophic lateral sclerosis to Drosophila larvae, wherein the larvae express a human TDP-43 transgene, and determining the survival of the larvae to an adult stage wherein the survival of the larvae indicates that the candidate therapeutic for amyotrophic lateral sclerosis is a therapeutic for amyotrophic lateral sclerosis.

Disclosed herein are a transgenic Drosophila comprising a human TDP-43 gene.

Disclosed herein are therapeutics for amyotrophic lateral sclerosis identified using a disclosed method.

### BRIEF DESCRIPTION OF THE FIGURES

FIG. 1 shows a schematic representation of Drosophila (dTDP-43) and human TDP-43 (huTDP-43). Protein domains are as indicated. NLS indicates Nuclear Localization Signal, NES indicates Nuclear Export Signal, RRM1 and RRM2 indicate RNA Recognition Motifs 1 and 2, and G-rich indicates Glycine rich domain (dark green).

FIG. 2 shows a schematic representation of the dTDP-43 gene located on the second chromosome. dTDP-43 gene structure, P-element insertions, predicted isoforms, RNAi target sequence (GD6943) and Df(2R)or-BR6 are as indicated.

FIGS. 3A-3D show a comparative analysis of dTDP-43 and wild-type larval NMJs. (A) shows a wild-type NMJ (w1118) labeled with the synaptic vesicle marker CSP (red) and the membrane marker HRP (green). Type 1b (arrowhead) and 1s (arrow) synaptic boutons. (B, C) are representative images of dTDP-43 mutant NMJs that show different degrees of reduced complexity compared to wild-type. (B) TBPH^{EY10530}/Df(2R)or-BR6 (EY10530/Df). (C) TBPH^{EY1053} homozygotes (EY10530). (D) Loss of dTDP-43 results in significantly less complex NMJs (Pᵥₐₗᵤₑ<0.05). Genotypes are as indicated. dTDP-43 bracket-loss of function alleles; RNAi bracket—RNAi lines driven by the motor neuron Gal4 driver c380 (31).

FIG. 4 shows that dTDP-43 was required for adult climbing behavior. The climbing index of dTDP-43 mutant adults (P(SUPor)TBPH^{KG08578}) was significantly altered during the 30 day trial period. These data represent averages of two independent trials in which >100 males were used per genotype. Similar results were obtained for the P(EPgy2)TBPH^{EY10530} allele.

FIG. 5 shows an amino acid sequence comparison between Drosophila (fly) and human (hu) TDP-43. RNA Recognition domains (RRM1 and RRM2) are shown in blue font. C-terminus domain is shown in green font (darker green corresponds to the Glycine-rich domain within the C-terminus). Amino acids that were mutated in human patients and conserved between the fly and human proteins are shown in red. Underlined residues correspond to human mutations in non-conserved amino acids. Asterisk marks the beginning of the C-terminus fragment that has been identified within cytoplasmic inclusions.

FIG. 6 shows that wild-type and various mutant forms of fly and huTDP-43 resulted in rough eyes accompanied by loss of pigmentation and necrosis. Genotypes were as indicated. Bottom right panel: Western blot probed for GFP and myc tags (top) shows various transgenes running at their predicted sizes: 85 kD for GFP-dTDP, 72 kD for huTDP-YFP and 33 kD for myc(6×)-dTDP Cterm. The asterisk indicates a smaller product of GFP-dTDP, possibly due to degradation. Tubulin was used as a loading control (bottom).

FIG. 7 shows wild-type and mutant TDP-43 aggregates in photoreceptor neurons. Larval eye imaginal discs showing the localization of various forms of TDP-43 as marked by GFP, mCherry or myc tags. Theus, the abnormal localization and the presence cytoplasmic (red arrows) and axonal aggregates (white arrows) were due to dTDP G298S and wild-type dTDP expression (compare to GFP NLS control). Genotypes are as indicated. Images shown are single confocal slices or 2-3 slice projections. Phalloidin marks cell shapes and axons. GFP and mCherry were imaged following a short fixation. Myc marks the C terminus domain, which appears entirely cytoplasmic (due to the lack of the NLS).

FIG. 8 shows wild-type and mutant TDP-43 aggregates in motor neurons. Ventral ganglia (top panels) showing the localization of various forms of TDP-43 as marked by GFP or mCherry tags. Bottom panels show high magnification view of midline motor neurons. Two different gains were used to show the abnormal perinuclear and cytoplasmic localization of wild-type dTDP and dTDP G298S in comparison to the GFP NLS control (red arrows). GFP and mCherry were imaged after a short fixation. Genotypes are as indicated.

FIG. 9 shows that mutant TDP-43 overexpression and TDP-43 RNAi but not the C terminus of dTDP-43 affected adult locomotor behavior. Climbing index measured as described (FIG. 4). Genotypes and testing days are as shown. Ten flies were used per genotype.

FIG. 10 shows a dominant modifier genetic screen in the eye identified two candidate genomic regions as TDP-43 interactors. Genotypes are as indicated. Suppression of necrosis and roughness by Of 4956 and 7144 occurred. Some loss of pigmentation remained.

FIG. 11 shows a drug screen for compounds that rescue TDP-43 mediated neurotoxicity in vivo. FDA approved drugs from the Prestwick collection (200 mM in DMSO) are mixed with yeast-cornmeal based food and a bromophenol blue (as a mixing indicator) at a final concentration of 50 μM. Larvae expressing TDP-43 (wild-type or mutant, as shown) are raised on drug containing food. In the absence of drugs, TDP-43 expression is lethal at the pupal/pharate stage. Drugs that rescued TDP-43 induced lethality (anywhere from 1-12 adults) were considered as candidates for therapeutic studies.

FIG. 12 shows that TDP-43 co-localizes with PABP in neuronal stress granules. Genotypes indicated on the left. Stainings indicated on the top. Arrowheads indicate granules containing both TDP-43 and PABP (right panels). TDP-43 was visualized via YFP tag. Cell bodies were saturated to allow visualization of the smaller RNA granules within neurites.

FIG. 13 shows that TDP-43 co-fractionated with insoluble complexes. Cellular fractionation of adult head tissue showed that TDP-43 was found in all cellular fractions including the soluble (LS, Low Salt), Triton-X100 (TX, nonionic detergent) as well as sarkosyl (SK, ionic detergent) and detergent insoluble fraction (U, urea). All TDP variants exhibited high molecular weight forms corresponding to posttranslational modifications (top arrowheads, full-length) as well as truncated C terminal fragments (bottom arrowhead, C-terminus). Tubulin was used as a loading control. TDP-43 transgenes used are C-terminal tagged with YFP. Western blot was performed using anti-GFP or tubulin antibodies as appropriate.

FIGS. 14A-14H show NMJ morphology and locomotor function were altered when TDP-43 was overexpressed in motor neurons. (A-F) D42 Gal4 driven TDP-43 resulted in decreased number of synaptic boutons (CSP, HRP, as indicated). (G) Quantification of synaptic boutons indicated a reduction in synapse size due to TDP-43 overexpression. (H) Larvae expressing TDP-43 in motor neurons exhibited a significant increase in larval turning time, a well-established behavioral assays used to determine locomotor function. Student's T test was used to calculate significance. *=Pᵥₐₗᵤₑ of <0.5; **=P_{value of}<0.01; ***=Pᵥₐₗᵤₑ of <0.001. Scale bar in (A): 30 μm.

FIGS. 15A-15H show that the ratio between presynaptic active zones (AZ) and post synaptic glutamate receptors was affected by motor neuron expression of TDP-43. (A-H) D42 driven TDP-43 preferentially increased the number of presynaptic active zones (AZ) per bouton (quantification in FIG. 15G) without a similar increase in postsynaptic GluR (except for wt TDP, FIG. 15H). Stainings as indicated. Student's T test was used to calculate significance. *=Pᵥₐₗᵤₑ<0.5; **=Pᵥₐₗᵤₑ<0.01; ***=Pᵥₐₗᵤₑ<0.001. Scale bar in (A): 5 μm

FIGS. 16A-16F show locomotor activity was reduced and sleep patterns were altered in adults expressing TDP-43. (A-F) TDP-43 variants (as shown) were expressed with D42 Gal4. Measurements using DAMs show that locomotor activity was significantly reduced overall (A) and total sleep was significantly altered (B). WT TDP-43 expressing flies slept overall longer than the mutant variants. During the day (C, D) as well as during nighttime (E, F), TDP-43 expression resulted in significantly more sleep episodes that last shorter amounts of time. To determine significance, a non-parametric Wilcox test was calculated in R. In these experiments, TDP-43 transgenics with lower levels of expression that do not affect adult viability were used. This is in contrast to the transgenics employed in the drug screen where high levels of expression lead to adult lethality.

FIG. 17 shows that the insulin signaling pathway modulated TDP-43's neurotoxicity in vivo. Constitutively active PI3K and S6K overexpression enhanced while Akt reduction by RNAi mitigated TDP-43 induced neurodegeneration. Genotypes as indicated. GMR Gal4 was used to drive expression in the retina. Overexpression of DP 110^{CAAX} and S6K^{STDETE} alone do not exhibit visible phenotypes (top row), indicating that the observed enhancements were synergistic and not additive.

FIG. 18 shows that the insulin signaling pathway and the molecular targets of candidate anti-diabetic drugs are conserved in Drosophila. Sulfonylureas target ATP-dependent potassium channels K_{ATP} comprised of Inner Rectifier Potassium channel lrk3 in association with the sulfonylurea receptor (Sur). As a result of K_{ATP} activation on beta cells, the pancreas underwent a surge in Ca²⁺ that mimics glucose entry and released more insulin. In Drosophila, there are seven insulin like peptides (shown as dILPs), which initiate a signaling cascade upon binding to the insulin receptor (lnR). For simplicity, only core components of the pathway are shown. Biguanides such as Metformin and Phenformin stimulate AMPK, an energy sensor, which in turn inhibits TOR activation. Shaded grey boxes indicate various cellular outcomes of the insulin/PI3K/Akt/TOR signaling.

FIG. 19 shows a summary of TDP-43 variant specific phenotypes in motor neurons and glia. TDP indicates wild type human TDP-43. D169G indicates a substitution from D to G at position 169 in human TDP-43. G298S indicates a substitution from G to S at position 298 in human TDP-43. A315T indicates a substitution from A to T at position 315 in human TDP-43. N345K indicates a substitution from N to K at position 345 in human TDP-43,

FIG. 20 shows a summary of TDP-43 phenotypes. TBPH indicates wild type Drosophila TDP-43. TBPH A315T indicates a substitution from A to T at position 315 in Drosophila TDP-43. hTDP-43 indicates wild type human TDP-43. hTDP-43 A315T indicates a substitution from A to T at position 315 in human TDP-43.

FIGS. 21A-21H, 21A′-21F′ show the overexpression of human wild-type and A315T mutant TDP-43 led to neurodegeneration in the adult retina. (A-F) show that the expression of hwt and hA315T resulted in normal surface phenotypes at 25° C. (B and C compared with A). Both hTDP-43 transgenes exhibited strong surface phenotypes when expressed at higher levels (29° C., see E and F) compared with controls (D). Genotypes are as indicated. Anterior right, dorsal up. (A′-F′) the corresponding plastic sections indicate that regardless of the surface phenotype, the retinas underwent cell loss. Note large areas of cell loss within the retina (arrows). Adult eyes shown are from 1-2-day-old flies. (G) is the western blot analyses showing hTDP-43 expression. Note similar levels of TDP-43 expression for the wild-type (hwt) and A315T (hA315T) transgenes as well as increased expression levels in an additional hA315T line (hA315T HE). Genotypes are as indicated, on top. Blotting antibodies are as indicated on the right. Tubulin was used as a loading control. (H) shows the quantification of relative protein levels from Western blot analysis. Scale bar (A′): 30 μm.

FIGS. 22A-22F, 22D′-22F′, 22D″-22F″ show that the overexpression of TDP-43 in larval eye imaginal discs led to altered cytoplasmic localization and axonal aggregates. (A-F) are single-confocal slices (1 mm each) showing hTDP-43 localization when expressed in the developing retina with GMR-Gal4. hTDP-43 visualized via individual fluorescent tags [as indicated (B, C and E-F″), compare with GFP-NLS (A, D-D″)]. Filamentous actin labeled with phalloidin (phall), and DNA stained with Hoechst (as indicated). Note TDP-43 aggregates in axons (arrowheads). (D′-F′) High magnification views of optic stalk show TDP-43 aggregates in axons (white arrowheads, D′-F′), whereas GFP-NLS remains restricted to nuclei (D′). (D″-F″) High-magnification insets showing the localization of GFP-NLS (D″) and TDP-43 wt and A315T in relation to the nucleus (E″-F″). Stainings as indicated. Note that hwt forms more pronounced aggregates than hA315T, which appears more diffusely distributed (asterisk). Individual nuclei are circled in red. Red arrowheads indicate some amount of depletion from the nucleus hwt. Scale bar (A): 50 μm.

FIGS. 23A-23E, 23A′-23E′, 23A″-23E″ show the subcellular localization of TDP-43 in motor neurons. (A-E) show D42-Gal4-driven expression of GFP-NLS (A, control) and TDP-43 variants (B-E) in ventral ganglia of third instar larvae. Genotypes are indicated on the top, and stainings are shown on the left. The images shown represent projections of 1 mm confocal slices. GFP (or YFP) and RFP indicate tags used to visualize TDP-43 variants, and DNA visualized using Hoechst. (A′-E″) High-magnification views of ventral ganglia shown in (A)-(E). Both human transgenes (wt and A315T) remained restricted to the nucleus (white arrows in B and B′, and C and C′). Note cell with peripheral nuclear localization in hTDP-43 wt (white arrowhead in B and B′). Wild-type TBPH translocated to the cytoplasm and forms axonal aggregates (red arrows in D′). The fly A315T mutant protein was mostly restricted to the nucleus (white arrows in E and E′). Hoechst staining of the samples is shown in (A′)-(E′) labels DNA. Scale bars: 30 μm in (A), 15 μm in (A′).

FIGS. 24A-24I, 24A′-24H′, 24A″-24H″ show that the NMJ morphology was altered by overexpression of TDP-43 variants. (A-H″) Larval NMJs at muscles 6/7, abdominal segment A3 (A-D″) and abdominal segment A6 (E-H″). Genotypes are shown on the left, and stainings are indicated on the top. Selected terminal type 1b boutons (marked with asterisks) are shown in (A′-D′) and (A′-D′, HRP only), and likewise for A6 in (E′-H′) and (E′-H′, HRP only). Arrowheads indicate thinning of the HRP-stained neuronal membrane. Arrows indicate satellite boutons. D42-Gal4-driven overexpression of TDP-43 (wild-type and A315T) affects various aspects of synaptic morphology [see (I) for quantitative analyses at A3, which include a high expressing hA315T transgene (A315T HE)]. t-test was used to determine statistical significance. ***P<0.001; **P<0.01; *P<0.05. Scale bar (A): 45 μm.

FIGS. 25A-25D show that the overexpression of TDP-43 variants acted as a dominant negative and affected locomotor activity and survival. (A) Larvae expressing D42-driven human wild-type, hA315T mutant TDP-43 (including hA315T HE) and TBPH RNAi transgenes (alone or in combination) took significantly longer to turn over following a ventral-up inversion. Genotypes are as indicated. (B) hTDP-43 expression or TBPH RNAi in motor neurons caused a dramatic decrease in adult survival. (C and D) Adult climbing assays performed on the adult survivors show severe motor impairment at both 18° C. (C) and 25° C. (D). Student's t-test was used to determine statistical significance. ***P<0.001; **P<0.01; *P<0.05.

FIGS. 26A-26G, 26A′-26D′, 26E1′-26E2′, 26E1″-26E2″, 26F′-26G′, 26F″-26G″ show the motor neuron apoptosis due to TDP-43 overexpression. (A-D) show TUNEL staining marks apoptotic cells. Genotypes/treatment are indicated on top, and stainings are shown on the left. (A and A′) RFP-NLS expressing ventral ganglia treated with HCl exhibits widespread TUNEL staining (B and B′) RFP-NLS expressing ventral ganglia shows no indication of cell death (negative control). (C and C′) Wild-type TBPH expression resulted in some apoptotic cells within the ventral ganglia (arrows, compare C′ with A′ and B′). (D and D′) Few apoptotic cells were detected in A315T TBPH expressing ventral ganglia (arrows). (E) GFP NLS expression in adult motor neurons using D42 Gal4. Thoracic segments T1, 2, 3 as shown. Arrows (E-G) indicate areas where motor neurons are located. Note: The region marked by red inset in (E) is shown at high magnification for different thoracic ganglia (assayed for apoptosis) in (E1′)-(G″). (E1′ and E1″) TUNEL staining in the T1/T2 region of a GFP-NLS adult thoracic ganglia treated with HCl (positive control). (E2′ and E2″) GFP-NLS expression does not induce apoptosis (negative control). (F′-G″) Adult thoracic ganglia expressing hwt (F′ and F″) and hA315T mutant (G′ and G″). Motor neurons visualized via fluorescent protein tag when either hwt (F) or hA315T mutant TDP-43 (G) is expressed with the D42 driver. Note the dramatic reduction in motor neurons as a result of hwt expression (F). TUNEL assays indicate the presence of apoptotic cells (arrows) when hwt (F′ and F″) or, to a lesser extent, A315T mutant hTDP-43 (G′ and G″) were expressed in adult motor neurons. Scale bar: 70 μm (A), 125 μm (E) and 70 μm (E1′).

FIGS. 27A-27K show that TDP-43 toxicity is modulated by the proteasome, HSP70 activities and the apoptosis pathway. (A and E) Compound eyes expressing hwt (A) or A315T mutant hTDP-43 (hA315T, E) at 25° C., in 15-day-old adults, exhibit age-dependent depigmentation. Genotypes are as indicated on the left, and interacting genes are on the top. All transgenes were expressed with GMR-Gal4. (B and F) Coexpression of pros 13 enhancedthe depigmentation phenotype due to TDP-43 overexpression (arrows). (C, D, G and H) Coexpression of Hsp70 (C and G) or the p35 caspase inhibitor (D and H) alleviated the eye depigmentation phenotypes due to TDP-43 overexpression. All comparisons were performed with similarly aged flies. (I-K) show that the overexpression of prosβ, Hsp70 or p35 alone does not lead to visible eye phenotypes.

FIGS. 28A-28Y show that the overexpression of mutant TDP-43 leads to age- and dose-dependent neurodegeneration in the adult retina. (A-L) GMR Gal4 expression of TDP-43 D169G (D-F), G298S (G-I) and N345K (G-L) variants resulted in age-dependent loss of pigment compared with GMR Gal4 driver controls (A-C) at 25° C. Note that D169G depigmentation was milder than in G298S and N345K variants. (M-X) TDP-43 variants D169G TDP-43 (P-R), G298S TDP-43 (S-U) and N345K TDP-43(V-X) exhibited stronger surface phenotypes, including necrosis, compared with controls (M-O) when expressed at higher levels (29° C.). Genotypes and ages are indicated. Anterior right, dorsal up. (Y) Western blot analyses showing TDP-43 expression in several transgenic lines. For each mutant variant multiple lines were characterized, two of which are shown. Genotypes are indicated on top; blotting antibodies are indicated on the left. Arrowheads on the right indicate full-length and C-terminus truncation of TDP-43. Tubulin was used as a loading control.

FIGS. 29A-29I show that TDP-43 forms axonal aggregates in developing eyes. (A-H) Confocal images (single slices, 1 μm each or 2-3 slice projections) showing GFP NLS (A,E) or TDP-43 variant (B-D, F-H) localization when expressed in the developing eye neuroepithelium with GMR-Gal4. TDP-43 visualized via individual fluorescent YFP tags (indicated as GFP). Filamentous actin labeled with phalloidin, DNA stained with Hoechst 33342 as indicated. Note TDP-43 puncta in axons (arrowheads in B-D, F-H and insets); compare with GFP NLS controls (arrowheads in A, E and inset). (I) Cellular fractionation of adult head tissue shows TDP-43 distribution complexes ranging from highly soluble (LS, low salt) to insoluble (U, urea). Triton X-100 (TX) and sarkosyl (SK) correspond to non-ionic and ionic detergent soluble fractions, respectively. TDP-43 was detected using anti-GFP antibodies. Tubulin was used as a loading control. Scale bar: 75 μm.

FIGS. 30A-30Y show that the TDP-43 expression in motor neurons or glia led to cytoplasmic aggregates and nuclear morphology defects. (A-F) TDP-43 expressed with D42 Gal4 was visualized using anti-GFP antibodies (B-F) and compared with D42 Gal4>GFP NLS controls (A). Motor neurons within the ventral ganglia were labeled by GFP; DNA was stained with Hoechst 33342. (G-I) Mutant variants but not wild-type TDP-43 alter nuclear shape by increasing eccentricity (G), decreasing the form factor (H) and increasing compactness (I) compared with D42 Gal4 driver controls. (J-P) TDP-43 expressed in glia with repo Gal4 was visualized using anti-GFP antibodies (K-P) and compared with repo Gal4>GFP NLS controls stained with Hoechst 33342 to visualize DNA (J). Note the presence of TDP-43 puncta in the cytoplasm (K-P). (Q-V) TDP-43 was expressed in puncta (arrows) within glial cells enveloping the neuromuscular junction synapse labeled by CSP and HRP. (W-Y) Glial expression caused relatively mild changes in nuclear shape, primarily in D169G and N345K compared with repo Gal4 driver controls. Eccentricity, form factor and compactness are shown as means±s.e.m.; ***P<0.001. Scale bars: 20 μm.

FIGS. 31A-31I show that TDP-43 is expressed in dynamic cytoplasmic puncta in primary motor neurons. (A-F) TDP-43 expressed using D42 Gal4, visualized with anti-GFP antibody, localizes to distinct puncta within neurites (arrows). Neuronal membranes were labeled with anti-HRP antibodies. (G) Velocity quantification of TDP-43-containing puncta (mean, maximum and minimum velocities). (H) Quantification of total and net distances for TDP-43 puncta. (I) FRAP indicates that TDP-43 is mobile within neurites. Note that wild-type kinetics were significantly different to those of mutant TDP-43 (see text for discussion). Values show means±s.e.m. Scale bar: 15 μm.

FIGS. 32A-32P show that TDP-43 variants affect the growth and function of the neuromuscular junction synapse. (A-F) Neuromuscular junctions in larvae expressing TDP-43 driven by D42 Gal4 were labeled by CSP (synaptic vesicle marker) and HRP (neuronal membrane marker). (G) Quantification of synaptic boutons indicates a reduction in synaptic size due to TDP-43 overexpression. (H) Larvae expressing TDP-43 in motor neurons were impaired in larval turning behavior. (I-N) Neuromuscular junctions in larvae expressing TDP-43 driven by repo Gal4 were labeled by CSP (synaptic vesicle marker) and HRP (neuronal membrane marker). (O) Quantification of synaptic boutons indicated an increase in synaptic size due to TDP-43 overexpression. (P) Larvae expressing TDP-43 in glia were impaired in larval turning behavior. Values show means±s.e.m. All comparisons were performed using D42 Gal4 driver controls and t-test was used to calculate significance; *P<0.5, **P<0.01, ***P<0.001. Scale bars: 30 μm.

FIGS. 33A-33P show that the active zones and postsynaptic glutamate receptor distribution were differentially affected at the neuromuscular junction by TDP-43 expression in motor neurons versus glia. (A-F) The presynaptic marker Bruchpilot (visualized with NC82 antibodies) and postsynaptic marker glutamate receptor (GluR) were used to label neuromuscular junctions in larvae expressing TDP-43 in motor neurons (staining and genotype as indicated). (G,H) Quantification of NC82 puncta indicated a significant increase in the number of presynaptic active zones per bouton (G), which was not accompanied by a similar increase in postsynaptic GluR (except for wild-type TDP, see H). (I-N) The presynaptic marker Bruchpilot (visualized with NC82 antibodies) and postsynaptic marker GluR label neuromuscular junctions in larvae expressing TDP-43 in glia (staining and genotype as indicated). (O,P) Quantification of NC82 puncta showed no effect on the number of presynaptic active zones per bouton (O), but a significant increase in postsynaptic GluR expression (P). Values show means±s.e.m. All comparisons were performed using D42 Gal4 driver controls and t-test was used to calculate significance; *P<0.5, **P<0.01, ***P<0.001. Scale bars: 5 μm.

FIGS. 34A-34L show that TDP-43 overexpression affected sleep and locomotor activity in adult flies. (A-F) TDP-43 variants were expressed in motor neurons using D42 Gal4. Measurements using DAMs showed that locomotor activity is significantly reduced overall (A) and total sleep is significantly altered, with wild-type TDP-43 leading to longer overall sleep than the mutant variants (B). During the day (C,D) as well as during the night (E,F), TDP-43 expression resulted in significantly more sleep episodes (D,F) that last shorter intervals of time (C,E). (G-L) TDP-43 variants were expressed in glial cells using repo Gal4. Measurements using DAMs show variable effects on locomotor activity (G) and a significant reduction in total sleep (H). During the day (I,J) there were variable effects on sleep. During the night (K,L), TDP-43 expression in glia results in significantly more sleep episodes (L) that last shorter amounts of time (K). Values show means±s.e.m. All comparisons were performed using D42 Gal4 driver controls and significance was determined using a non-parametric Wilcox test calculated in R; *P<0.5, **P<0.01, ***P<0.001.

FIG. 35 shows the structure of Pioglitazone.

FIG. 36 shows the structure of Troglitazone.

FIGS. 37A-37G show that pioglitazone rescued several aspects of TDP-43 dependent toxicity in motor neurons. (A) Drug screen strategy. Overexpression of TDP-43 (wild type or mutants) in motor neurons was lethal at the pupal or pharate adult stage. In screening the Prestwick collection of FDA approved drugs, this phenotype was used to identify adult survivors with fully extended wings (rescue of lethality). (B, C) Pioglitazone rescued TDP-43 dependent pupal lethality. Wild type TDP-43 (TDP^{WT}, B) or disease associated G298S mutant (TDP^{G298S}, C) expressed in motor neurons using D42-Gal4 driver result in >95% pupal lethality. D42>TDP larvae were grown on fly food containing different concentrations of Pioglitazone or DMSO (as indicated). Surviving adults were normalized to pupae numbers and plotted on the Y axis, as shown. Error bars indicate SEM. (D, E) Pioglitazone rescued larval locomotor defects caused by TDP-43 overexpression in motor neurons. D42>TDP^{WT} (D) and D42>TDP^{G298S} (E) Larvae grown on fly food containing different concentrations of Pioglitazone or DMSO (as indicated) were assayed for locomotor function using larval turning assays. Note that at the concentration of 1 μM, pioglitazone significantly rescued locomotor dysfunctions for both TDP^{WT} and TDP^{G298S}. Error bars indicate SEM. Student's T test was used to assess statistical significance. ***—Pᵥₐₗᵤₑ<0.001. (F, G) Pioglitazone did not improve lifespan of TDP-43 expressing flies. 1 μM pioglitazone was administered during development (F) or to adults (G) as indicated. Kaplan-Meier survival analysis shows no significant change in lifespan under either condition when compared to DMSO-fed controls.

FIGS. 38A-38D show that TDP-43 dependent toxicity in glia, but not in muscles, was partially mitigated by pioglitazone (A-B) Larval locomotor defects were mitigated by Pioglitazone when TDP-43 is expressed in glia, but not in muscles. TDP-43 (wild type or mutant, as indicated) was expressed in glia using Repo-Gal4 driver (A), or in muscles using BG487-Gal4 driver (B). Turning assay on larvae raised on fly food containing 1 μM pioglitazone or DMSO shows that pioglitazone can improve glial toxicity, but not that in muscles. Error bars indicate SEM. Student's T test was used to assess statistical significance. *—Pᵥₐₗᵤₑ<0.05. (C-D) Pioglitazone did not improve survival of Repo>TDP flies. 1 μM Pioglitazone was administered during development (C) or to adults (D) as indicated. Note that Kaplan-Meier survival analysis shows that developmentally administered pioglitazone results in significantly reduced lifespan for TDP^{WT} flies (C), while not affecting the lifespan under any other condition. *** indicates Pᵥₐₗᵤₑ<0.001.

FIGS. 39A-39D show that pioglitazone partially improved toxicity caused by FUS, but not SOD1 expression in motor neurons. (A-B) Larval locomotor defects caused by FUS, but not SOD1 expression in motor neurons, were improved by pioglitazone. Human FUS (A) and SOD1 (B), wild type or mutant (as indicated) were expressed in motor neurons using D42-Gal4 driver. Note that 1 μM pioglitazone only improved FUS-dependent larval locomotor defects, but not that caused by SOD1. Error bars indicate SEM. Student's T test was used to assess statistical significance. ** and *** indicate Pᵥₐₗᵤₑₛ<0.01 and <0.001, respectively. (C-D) Pioglitazone did not improve survival of D42>FUS flies. 1 M Pioglitazone was administered during development (C) or to adults (D) as indicated. Note that Kaplan-Meier survival analysis shows that developmentally administered pioglitazone results in significantly reduced lifespan for FUS^{WT} flies (C), while not affecting the lifespan under any other condition. * indicates Pᵥₐₗᵤₑ<0.05.

FIGS. 40A-40B show that E75 and E78 the Drosophila PPAR homologs are targets of pioglitazone. Flies expressing TDP^{WT} or TDP^{G298S} were crossed with E75 LOF (A) or E78 RNAi (B) virgin females on fly food containing 1 μM pioglitazone or DMSO (as indicated). Larval turning assay was done on larvae expressing TDP-43 or E75 LOF/E78 RNAi alone or together. Pioglitazone rescued TDP-dependent neurotoxicity, which was abolished in the context of E75 LOF and E78 RNAi. Error bars indicate SEM. Student's T test was used to assess statistical significance. *** and * indicate Pᵥₐₗᵤₑₛ<0.001 and <0.05, respectively.

FIGS. 41A-41D shows the metabolites altered by pioglitazone in the context of TDP^{WT}. N-acetylglutamine (A), Pyruvate (B), 4-hydroxybutyrate (C) and ALCAR (D) are shown as examples of restoration, restoration trend, worsening and no change by pioglitazone, respectively (see also FIG. 43). Genotypes and experimental conditions, as indicated. DMSO—vehicle control, PGZ—pioglitazone containing food.

FIG. 42 is a proposed model for the neuroprotective effect of pioglitazone. Pioglitazone (or Thiazolidinediones, in general) activated the nuclear receptor PPARγ homolog E75/E78 in vivo in Drosophila. This neuroprotection was imparted to TDP-43 and FUS, but not SOD1 fly models of ALS. Furthermore, the improvement was tissue-specific. In the model disclosed herein, some but not all aspects of cellular metabolism were restored by pioglitazone while others were worsened. Additionally, TDP^{WT} and TDP^{G298S} exhibited distinct metabolic alterations, implying differential mechanisms of disease pathophysiology.

FIG. 43 shows the effect of pioglitazone on TDP^{WT} specific metabolic alterations. Summary of pioglitazone specific biochemical alterations (TDP^{WT}-PGZ/TDP^{WT}-DMSO) shared with TDP^{WT} larvae (TDP^{WT}-RF/w¹¹¹⁸-RF). Metabolic pathways, biochemicals, experimental conditions, and restoration, no change or worsening effects, as indicated. Biochemicals altered by DMSO alone (TDP^{WT}-DMSO/TDP^{WT}-RF), as noted. Red and green colored cells indicate statistically significant means that are increased and decreased, respectively. Light red and light green colored cells indicate upward or downward trends, respectively, that although do not reach statistical significance but were considered in the analyses. N-acetyl glutamine was significantly restored by pioglitazone, while 9 other biochemical showed trends towards restoration. The changes for four metabolites were not ascertained due to confounding DMSO effects. Eight metabolites remained unchanged by pioglitazone treatment, with six being accompanied by DMSO effects, which made the interpretation difficult. Finally, six metabolites showed a trend towards worsening, as indicated. PGZ—pioglitazone containing food, DMSO—vehicle control, RF—regular food.

FIG. 44 shows the effect of pioglitazone on TDPG298S specific metabolic alterations. Summary of pioglitazone specific biochemical alterations (TDPG298S-PGZ/TDPG298S-DMSO) shared with TDPG298S larvae (TDPG298S-RF/w1118-RF). Metabolic pathways, biochemicals, experimental conditions, restoration and no change, as indicated. No biochemical were altered by DMSO alone (TDPG298S-DMSO/TDPG298S-RF). Red and green colored cells indicate statistically significant means that are increased and decreased, respectively. Light red and light green colored cells indicate upward or downward trends, respectively. Six biochemicals showed trends towards restoration and four metabolites remained unchanged by pioglitazone treatment. No metabolites showed any worsening effects. PGZ—pioglitazone containing food, DMSO—vehicle control, RF—regular food.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

### A. Disclosed Compositions and Methods

Disclosed herein are compositions useful in an in vivo method of screening for a therapeutic for amyotrophic lateral sclerosis. Disclosed herein are compositions useful in methods of administering a candidate therapeutic for amyotrophic lateral sclerosis to Drosophila larvae. Disclosed herein are compositions useful in a method of determining the survival of the larvae to an adult stage when administered a candidate therapeutic. Disclosed herein are compositions useful in developing, creating, and utilizing transgenic Drosophila. For example, disclosed herein are transgenes used in Drosophila. In an aspect, a transgenic Drosophila can comprise a human TDP-43 gene. In an aspect, a transgenic Drosophila can comprise a human TDP-43 gene comprising one or more mutations. In an aspect, a transgenic Drosophila can comprise a human TDP-43 gene comprising one or more substitutions. In an aspect, one or more mutations of a human TDP-43 transgene can correspond to a D169G, G298S, A315T, or N345K amino acid substitution.

Disclosed herein are methods for screening for a therapeutic for amyotrophic lateral sclerosis. In an aspect, disclosed is an in vivo method of screening for a therapeutic for amyotrophic lateral sclerosis, the method comprising administering a candidate therapeutic for amyotrophic lateral sclerosis to Drosophila larvae, wherein the larvae express a human TDP-43 transgene, and determining the survival of the larvae to an adult stage, wherein the survival of the larvae indicates that the candidate therapeutic for amyotrophic lateral sclerosis is a therapeutic for amyotrophic lateral sclerosis. In an aspect, the larvae can express a human TDP-43 transgene in one or more motor neurons. In an aspect, a human TDP-43 transgene can comprise one or more substitutions. In an aspect, a human TDP-43 transgene can comprise one or more mutations. In an aspect, one or more mutations in a human TDP-43 transgene can correspond to a D169G, G298S, A315T, or N345K amino acid substitution. In an aspect, a mutation can correspond to a D169G substitution. In an aspect, a mutation can correspond to a G298S substitution. In an aspect, a mutation can correspond to a A315T substitution. In an aspect, a mutation can correspond to a N345K substitution.

Disclosed are in vivo methods of screening for a therapeutic for amyotrophic lateral sclerosis, the method comprising administering a candidate therapeutic for ALS to Drosophila larvae, wherein the larvae express a human TDP-43 transgene; and assaying for amelioration of one or more sign or symptom associated with ALS, wherein the amelioration of one or more sign or symptom indicates that the candidate therapeutic for ALS is a therapeutic for ALS.

Assaying for amelioration of one or more sign or symptom associated with ALS can involve assaying for the sign or symptom prior to administration of a candidate therapeutic and assaying for amelioration of the sign or symptom after administration of a candidate therapeutic, wherein amelioration of the sign or symptom after administration compared to the sign or symptom prior to administration indicates that the candidate therapeutic for ALS is a therapeutic for ALS.

Signs or symptoms associated with ALS can be phenotypic or genotypic. In some aspects, phenotypic signs or symptoms can include, but are not limited to the presence of ketone bodies, such as, 4-hydroxybutyrate (GHB) or pyruvate. Amelioration, or a decrease, in ketone bodies or pyruvate can indicate that a candidate therapeutic for ALS is a therapeutic for ALS. Therefore, disclosed are in vivo methods of screening for a therapeutic for amyotrophic lateral sclerosis, the method comprising administering a candidate therapeutic for ALS to Drosophila larvae, wherein the larvae express a human TDP-43 transgene; and assaying for the presence of ketone bodies, wherein a decrease in ketone bodies indicates that the candidate therapeutic for ALS is a therapeutic for ALS.

Disclosed are in vivo methods of screening for a therapeutic for amyotrophic lateral sclerosis, the method comprising administering a candidate therapeutic for ALS to Drosophila larvae, wherein the larvae express a human TDP-43 transgene; and assaying for amelioration of one or more sign or symptom associated with ALS, wherein the amelioration of one or more sign or symptom indicates that the candidate therapeutic for ALS is a therapeutic for ALS, wherein assaying for amelioration of one or more sign or symptom associated with ALS comprises examining neuromuscular junctions, characterizing locomotion, quantifying motor neuron cell death, examining eye neuroepithelium, identifying cytoplasmic inclusions, or a combination thereof.

Disclosed herein are therapeutics for amyotrophic lateral sclerosis. In an aspect, a disclosed therapeutic can ameliorate one or more signs or symptoms associated with amyotrophic lateral sclerosis. In an aspect, one or more signs or symptoms associated with amyotrophic lateral sclerosis can comprise phenotypic signs or symptoms. In an aspect, a disclosed method for screening for a therapeutic for amyotrophic lateral sclerosis can comprise one of the following: examining neuromuscular junctions, characterizing locomotion, quantifying motor neuron cell death, examining eye neuroepithelium, and identifying cytoplasmic inclusions. In an aspect, a disclosed method for screening for a therapeutic for amyotrophic lateral sclerosis can comprise a combination of the following: examining neuromuscular junctions, characterizing locomotion, quantifying motor neuron cell death, examining eye neuroepithelium, and identifying cytoplasmic inclusions.

Disclosed herein are Drosophila models of amyotropic lateral sclerosis. In an aspect, Drosophila models of amyotropic lateral sclerosis comprise a human TDP-43 gene. In an aspect of the disclosed Drosophila models of amyotropic lateral sclerosis comprising a human TDP-43 gene, TDP-43 can exhibit functional interactions with one or more components of the insulin signaling pathway. Components of the insulin signaling pathway can include, but are not limited to, the following: InR (Insulin Receptor), PI3K (PI3 Kinase), PTEN (phosphatase and tensin homolog), PDK1 (phospho-inositide kinase 1), Akt, FoxO, SIRT (sirtuin), TSC1, 2 (tuboerous sclerosis 1, 2), GSK (glycogen synthase kinase), MAPK, AMPK, TOR (Target of rapamycin), Rictor, Raptor, S6K (S6 kinase), 4E-BP1 (eukaryotic translation initiation factor 4E binding protein 1), EIF4A (eukaryotic translation initiation factor 4A), EIF4E (eukaryotic translation initiation factor 4E), EIF2 (eukaryotic translation initiation factor 2), PPAR gamma, and FABP (Fatty acid binding protein).

For example, in an aspect, TDP-43 can interact in vivo with InR (Insulin Receptor), PI3K (PI3 Kinase), PTEN (phosphatase and tensin homolog), PDK1 (phospho-inositide kinase 1), Akt, FoxO, SIRT (sirtuin), TSC1, 2 (tuberous sclerosis 1, 2), GSK (glycogen synthase kinase), MAPK, AMPK, TOR (Target of rapamycin), Rictor, Raptor, S6K (S6 kinase), 4E-BP1 (eukaryotic translation initiation factor 4E binding protein 1), EIF4A (eukaryotic translation initiation factor 4A), EIF4E (eukaryotic translation initiation factor 4E), EIF2 (eukaryotic translation initiation factor 2), PPAR gamma, or FABP (Fatty acid binding protein).

In an aspect, TDP-43 can interact in vivo with a combination of one or more of InR (Insulin Receptor), PI3K (PI3 Kinase), PTEN (phosphatase and tensin homolog), PDK1 (phospho-inositide kinase 1), Akt, FoxO, SIRT (sirtuin), TSC1, 2 (tuboerous sclerosis 1, 2), GSK (glycogen synthase kinase), MAPK, AMPK, TOR (Target of rapamycin), Rictor, Raptor, S6K (S6 kinase), 4E-BP1 (eukaryotic translation initiation factor 4E binding protein 1), EIF4A (eukaryotic translation initiation factor 4A), EIF4E (eukaryotic translation initiation factor 4E), EIF2 (eukaryotic translation initiation factor 2), PPAR gamma, and FABP (Fatty acid binding protein).

In an aspect, an interaction between TDP-43 and one or more of the components of the insulin signaling pathway can be sequential. In an aspect, an interaction between TDP-43 and one or more of the components of the insulin signaling pathway can be concurrent or concomitant. In an aspect, some of the interactions between TDP-43 and one or more of the components of the insulin signaling pathway can be sequential and some of the interactions between TDP-43 and one or more of these components can be concurrent or concomitant. In an aspect, such interactions can occur once or can occur repeatedly.

### 1. DEFINITIONS

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the amino acid abbreviations are conventional one letter codes for the amino acids and are expressed as follows: A, alanine; B, asparagine or aspartic acid; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine.

“Peptide” as used herein refers to any peptide, oligopeptide, polypeptide, gene product, expression product, or protein. For example, a peptide can be an enzyme. A peptide is comprised of consecutive amino acids. The term “peptide” encompasses naturally occurring or synthetic molecules.

In general, the biological activity or biological action of a peptide refers to any function exhibited or performed by the peptide that is ascribed to the naturally occurring form of the peptide as measured or observed in vivo (i.e., in the natural physiological environment of the protein) or in vitro (i.e., under laboratory conditions).

The term “enzyme” as used herein refers to any peptide that catalyzes a chemical reaction of other substances without itself being destroyed or altered upon completion of the reaction. Typically, a peptide having enzymatic activity catalyzes the formation of one or more products from one or more substrates. Such peptides can have any type of enzymatic activity including, without limitation, the enzymatic activity or enzymatic activities associated with enzymes such as those disclosed herein.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the terms “transformation” and “transfection” mean the introduction of a nucleic acid, e.g., an expression vector, into a recipient cell including introduction of a nucleic acid to the chromosomal DNA of said cell. The art is familiar with various compositions, methods, techniques, etc. used to effect the introduction of a nucleic acid into a recipient cell. The art is familiar with such compositions, methods, techniques, etc. for both eukaryotic and prokaryotic cells. The art is familiar with such compositions, methods, techniques, etc. for the optimization of the introduction and expression of a nucleic acid into and within a recipient cell.

As used herein, the term “subject” refers to the target of administration, e.g., an animal. In an aspect, the subject of the herein disclosed methods can be a Drosophila. In an aspect, the subject of the herein disclosed methods can be mammal, a fish, a bird, a reptile, or an amphibian Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, the subject is a mammal. A patient refers to a subject afflicted with a disease or disorder. The term “patient” includes human and veterinary subjects. In some aspects of the disclosed methods, the subject has been diagnosed with a need for treatment for amyotrophic lateral sclerosis, such as, for example, prior to the administering step.

As used herein, the term “treatment” refers to the medical management of a subject or a patient with the intent to cure, ameliorate, stabilize, or prevent a disease, pathological condition, or disorder, such as, for example, ALS. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as ALS). In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is amyotrophic lateral sclerosis.

As used herein, the term “prevent” or “preventing” refers to precluding, averting, obviating, forestalling, stopping, or hindering something from happening, especially by advance action. In an aspect, prevent or preventing refers to the ameliorating of one or more signs and symptoms associated with ALS. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by compositions or methods disclosed herein. For example, “diagnosed with amyotrophic lateral sclerosis” means having been subjected to a physical examination by a person of skill, for example, a physician, and found to have a condition that can be diagnosed or treated by a compound or composition that alleviates or ameliorates one or more symptoms associated with amyotrophic lateral sclerosis.

As used herein, the phrase “identified to be in need of treatment for a disorder,” or the like, refers to selection of a subject based upon need for treatment of the disorder. For example, a subject can be identified as having a need for treatment of a neurodegenerative disorder (e.g., amyotrophic lateral sclerosis) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. In an aspect, administering can refer to oral administration, such as, in food. Administration can be continuous or intermittent. In various aspects, a preparation can be administered therapeutically; that is, administered to treat an existing disease or condition. In further various aspects, a preparation can be administered prophylactically; that is, administered for prevention of a disease or condition, such as, for example, amyotrophic lateral sclerosis.

The term “contacting” as used herein refers to bringing a disclosed compound and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the term “determining” can refer to measuring or ascertaining a quantity or an amount or a change in expression and/or activity level, e.g., of a nucleotide or transcript or polypeptide. For example, determining the amount of a disclosed transcript or polypeptide in a sample as used herein can refer to the steps that the skilled person would take to measure or ascertain some quantifiable value of the transcript or polypeptide (i.e., TDP-43) in the sample. The art is familiar with the ways to measure an amount of the disclosed nucleic acids, transcripts, polypeptides, etc.

As used herein, the term “level” refers to the amount of a target molecule in a sample, e.g., a sample from a subject. The amount of the molecule can be determined by any method known in the art and will depend in part on the nature of the molecule (i.e., gene, mRNA, cDNA, protein, enzyme, etc.). The art is familiar with quantification methods for nucleotides (e.g., genes, cDNA, mRNA, etc.) as well as proteins, polypeptides, enzymes, etc. It is understood that the amount or level of a molecule in a sample need not be determined in absolute terms, but can be determined in relative terms (e.g., when compares to a control (i.e., a non-affected or healthy subject or a sample from a non-affected or healthy subject) or a sham or an untreated sample).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

By “modulate” is meant to alter, by increase or decrease. As used herein, a “modulator” can mean a composition that can either increase or decrease the expression level or activity level of a gene or gene product such as a peptide. In an aspect, a peptide can be TDP-43. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition.

As used herein, “EC₅₀,” is intended to refer to the concentration or dose of a substance (e.g., a compound or a drug) that is required for 50% enhancement or activation of a biological process, or component of a process, including a protein, subunit, organelle, ribonucleoprotein, etc. EC₅₀ also refers to the concentration or dose of a substance that is required for 50% enhancement or activation in vivo, as further defined elsewhere herein. Alternatively, EC₅₀ can refer to the concentration or dose of compound that provokes a response halfway between the baseline and maximum response. The response can be measured in an in vitro or in vivo system as is convenient and appropriate for the biological response of interest. For example, the response can be measured in vitro using cultured motor neuron cells or in an ex vivo organ culture system with isolated motor neuron cells. Alternatively, the response can be measured in vivo using an appropriate research model such as a Drosophila or rodent, including mice and rats. The Drosophila or rodent can be an inbred strain with phenotypic characteristics of interest such as, for example, obesity or diabetes. As appropriate, the response can be measured in a transgenic or knockout Drosophila or rodent, wherein a gene or genes has been introduced or knocked-out, as appropriate, to replicate a disease process (such as amyotrophic lateral sclerosis).

As used herein, “IC₅₀,” is intended to refer to the concentration or dose of a substance (e.g., a compound or a drug) that is required for 50% inhibition or diminution of a biological process, or component of a process, including a protein, subunit, organelle, ribonucleoprotein, etc. IC₅₀ also refers to the concentration or dose of a substance that is required for 50% inhibition or diminution in vivo, as further defined elsewhere herein. Alternatively, IC₅₀ also refers to the half maximal (50%) inhibitory concentration (IC) or inhibitory dose of a substance. The response can be measured in an in vitro or in vivo system as is convenient and appropriate for the biological response of interest. For example, the response can be measured in vitro using cultured motor neuron cells or in an ex vivo organ culture system with isolated motor neurons. Alternatively, the response can be measured in vivo using an appropriate research model such as a Drosophila or a rodent, including mice and rats. The mouse or rat can be an inbred strain with phenotypic characteristics of interest such as, for example, obesity or diabetes. As appropriate, the response can be measured in a transgenic or knockout Drosophila or rodent, wherein a gene or genes has been introduced or knocked-out, as appropriate, to replicate a disease process (i.e., ALS).

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Disclosed are the components to be used to prepare a composition of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

The present invention can be understood more readily by reference to the following detailed description of the invention and the Examples included therein.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### B. Examples

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### A. Amyotrophic Lateral Sclerosis (ALS)

ALS is a progressive neurodegenerative disorder that affects motor neurons and leads to paralysis and respiratory failure followed by death within 2-5 years of diagnosis. ALS is the third most common form of neurodegenerative cause of adult death after Alzheimer's and Parkinson diseases (1). ALS is a devastating fatal neurodegenerative disorder for which there is no cure. With a lifetime risk of dying from ALS currently estimated at 1/400-1/1,000 (1), any insights into the genetic pathways and potential therapies are likely to improve the lives of millions and alleviate the immeasurable public health issues associated with this devastating disease.

Annually, over 30,000 patients are afflicted with ALS in the United States. About 20% of all ALS patients also exhibit FrontoTemporal Degeneration (Banks et al., 2008). Familial ALS (fALS) affects 10% of patients and has been linked to several genes, the most common of which is C90RF72 (DeJesus-Hernandez et al., 2011; Renton et al., 2011). The remaining 90% of ALS cases are sporadic (sALS) and remain poorly understood, although some loci have been linked to both fALS and sALS (Alexander et al., 2002). These include C90RF72, SOD1, alsin, senataxin, VAMP/synaptobrevin-associated protein B, P150 dynactin, angiogenin, Fused in Sarcoma (FUS), and TAR DNA Binding Protein (TDP-43) (Beleza-Meireles and Al-Chalabi, 2009; Lagier-Tourenne and Cleveland, 2009). Based on the known functions of these loci and extensive phenotypic studies in model organisms, ALS appears to be the result of defects in several cellular processes and is accompanied by dysregulation of energy homeostasis (Beleza-Meireles and Al-Chalabi, 2009; Lagier-Tourenne and Cleveland, 2009). There is no cure for ALS.

Significant efforts have been made by numerous laboratories in the past several years to study ALS, primarily by using SOD1 models, which are representative of less than 1% of ALS cases. Thus, candidate therapeutic compounds selected mainly using SOD1 models have failed to identify therapeutically active compounds in human populations. This highlights the idea that other genetic and environmental mechanisms are involved in the pathology of ALS, including TDP-43, which is linked to a significant fraction of the patient population. As described herein, Drosophila model based on TDP-43 bears remarkable similarities to ALS and the in vivo drug screen has already generated therapeutic leads. The development of therapeutic strategies for ALS demands a concerted effort on both basic and translational research fronts. While traditionally there has been a low interest on the part of pharmaceutical companies, there is a renewed effort in tackling “rare disease” such as ALS.

### b. TDP-43

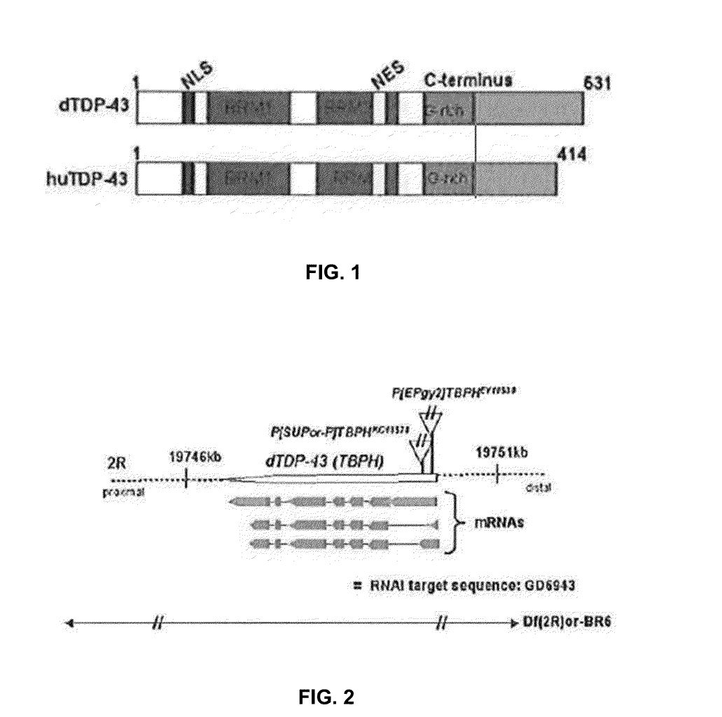

TDP-43 is a conserved RNA binding protein involved in several cellular processes. TDP-43 has been initially identified as a nucleic acid binding protein that regulates HIV gene expression by binding to its TAR DNA element, hence its original name, TAR DNA Binding Protein (TARDP, (18)). TDP-43 is ubiquitously expressed and co-localizes with SMN proteins in the nucleus. Its cellular functions include transcriptional repression, splicing, miRNA biogenesis, apoptosis and cell division (1). TDP-43 associates with RNA granules and co-purifies with β-actin and CaMKII mRNAs in cultured neurons. TDP-43 co-localizes with Fragile X protein and Staufen in an activity dependent manner, indicating that it can regulate synaptic plasticity in vivo by controlling the transport and splicing of synaptic mRNAs (19). TDP-43 protein consists of two RNA recognition motifs (RRM1 and 2) as well as a Glycine-rich domain within the C terminus (FIG. 1 and ref 20). In vitro assays have demonstrated that TDP-43 binds with high affinity UG-rich sequences, consistent with its role in mRNA splicing (21).

In an aspect, human wild-type TDP-43 can have the amino acid sequence: MSEYIRVTED ENDEPIEIPS EDDGTVLLST VTAQFPGACG LRYRNPVSQC MRGVRLVEGI LHAPDAGWGN LVYVVNYPKD NKRKMDETDA SSAVKVKRAV QKTSDLIVLG LPWKTTEQDL KEYFSTFGEV LMVQVKKDLK TGHSKGFGFV RFTEYETQVK VMSQRHMIDG RWCDCKLPNS KQSQDEPLRS RKVFVGRCTE DMTEDELREF FSQYGDVMDV FIPKPFRAFA FVTFADDQIA QSLCGEDLII KGISVHISNA EPKHNSNRQL ERSGRFGGNP GGFGNQGGFG NSRGGGAGLG NNQGSNMGGG MNFGAFSINP AMMAAAQAAL QSSWGMMGML ASQQNQSGPS GNNQNQGNMQ REPNQAFGSG NNSYSGSNSG AAIGWGSASN AGSGSGFNGG FGSSMDSKSS GWGM (SEQ ID NO:1). In an aspect, human wild-type TDP-43 can have the amino acid sequence set forth by GenBank Accesssion No. NP_{—}031401, AAH95435.1, or AAH71657.1, for example.

Pathological studies have identified the RNA binding protein TDP-43 as a component of cytoplasmic aggregates in neurons, glia and muscles, in ALS as well as Fronto-Temporal Lobar Degeneration, Alzheimer's and Inclusion Body Myositis (Maekawa et al., 2009; Neumann et al., 2006; Tan et al., 2007; Weihl et al., 2008). TDP-43's cellular functions are complex and reflect its ability to regulate several RNA targets at the level of splicing, transport and translation (Freibaum et al., 2010; Polymenidou et al., 2011; Sephton et al., 2011; Tollervey et al., 2011). Recently, several TDP-43 mutations have been identified in ALS patients (Kabashi et al., 2008; Rutherford et al., 2008; Sreedharan et al., 2008). Given its presence in cytoplasmic inclusions and the identification of several mutations linked to motor neuron degeneration, TDP-43 has emerged as a common denominator for a significant fraction of ALS cases known to date (Banks et al., 2008; Neumann, 2009). TDP-43's ability to form aggregates, induce apoptosis and splicing function can be used as secondary assays.

Most TDP-43 mutations found in ALS patients represent amino acid substitutions that are thought to increase TDP-43 phosphorylation and target it for degradation (Kabashi et al., 2008; Rutherford et al., 2008; Sreedharan et al., 2008). Evidence has been provided that these missense mutations mimic a loss of function for TDP-43 and that the RNA binding domain is required to mediate neurotoxicity (Estes et al., 2011; Feiguin et al., 2009; Voigt et al., 2010). Given its presence in cytoplasmic inclusions and the identification of several mutations linked to neural degeneration, TDP-43 has emerged as a common denominator for the majority of ALS cases known to date (Banks et al., 2008; Neumann, 2009). Thus studies using this model of ALS based on TDP-43 can provide widely applicable insights into this devastating disease.

In recent years, the fruit fly Drosophila has emerged as a premiere genetic model for studying human disease. Using loss of function mutations in Drosophila TDP-43 (dTDP-43) as well as RNAi knock-down approaches, alterations were identified in the architecture of the larval neuromuscular junction, locomotor defects and retina neurodegeneration in adults. To further establish a Drosophila model, transgenics were generated expressing wild-type and mutant forms of fly and human TDP-43, which mimic the mutations found in human patients. Experiments show that overexpression of TDP-43 results in the formation of cytoplasmic aggregates, neuronal loss and locomotor defects. Taken together, these data indicate that loss of function for TDP-43 as well as overexpression of wild-type and mutant TDP-43 in Drosophila recapitulate several aspects of the disease pathology. Furthermore, immunolocalization and genetic rescue data indicate a loss of function mechanism for the disease. The defects due to altered TDP-43 function can be corrected by genetic and pharmacological intervention and the genes/compounds that rescue the TDP-43 phenotypes can provide new therapeutic approaches for ALS. Genetic screens have identified a number of candidate genomic regions that rescue the retinal degeneration due to TDP-43 misexpression in the eye.

Since TDP-43 is involved in the pathology of a majority of ALS cases and has recently been shown to also be a cause for the disease, studies of a TDP-43 based model can provide insights into a wide spectrum of ALS cases. There is data indicating that in flies, alterations in TDP-43 function lead to defects in neuromuscular junction architecture, adult locomotion and neurodegeneration. Several transgenic lines expressing wild-type and mutant forms of dTDP-43 and human TDP-43 (huTDP-43) that correspond to mutations found in human patients have been developed. Overexpression of these transgenes leads to the formation of cytoplasmic inclusions and leads to neuronal dysfunction and death. The data demonstrates that the fly model recapitulates several aspects of ALS pathology. Genetic screens can identify single gene mutations that can rescue or enhance the TDP-43 phenotypes. These genes can provide insights into the molecular mechanisms involved in ALS and may represent novel therapeutic targets. In addition, this approach has the potential to identify ALS loci that are yet to be discovered in human patients. TDP-43-based phenotypes can be used to screen for compounds that rescue the neuroanatomical and functional defects in the fly model.

There is data indicating that ALS can be successfully modeled in Drosophila using both loss of function and gain of function approaches. Transgenic lines, wild-type and several mutant forms of both the fly and human proteins, which can be expressed specifically in motor neurons and the eye neuroepithelium using the Gal4-UAS system have been produced. Phenotypic analyses including testing for cytoplasmic inclusions, neuroanatomical and functional studies can determine the extent to which different alterations in TDP-43 recapitulate the pathology associated with the human disease.

### c. Drosophila as a Model for Amyotrophic Lateral Sclerosis

Drosophila has proven to be an excellent model for neurodegenerative disorders (23-26). Recently, a fALS model has been developed in Drosophila using overexpression of wild-type and two mutant forms of human SOD1 (huSOD1), namely A4V and G85R that have been linked to rapid disease progression (27). Over-expression of wild-type and mutant huSOD1 in motor neurons led to progressive climbing defects in adults with no effect on survivorship. Drosophila SOD1 (dSOD1) overexpression had no effect on climbing, indicating that hSOD1 can be intrinsically toxic to motor neurons. Further phenotypic analyses indicated that huSOD1 overexpression leads to the formation of ubiquitin negative cytoplasmic inclusions that can account for the synaptic transmission defects in the giant fiber circuit. No retinal degeneration was observed in adults expressing either wild-type or mutant huSOD1. Taken together, these data indicate that the expression of huSOD1 in Drosophila recapitulates some, but not all, of the SOD1-linked ALS (27).

A Drosophila model of ALS has been developed based on TDP-43 (Estes et al., 2011). Remarkably, the expression of human TDP-43 mutations, which mimic those found in ALS, in the Drosophila nervous system leads to neuroanatomical and functional defects that resemble those reported in patients (Estes et al, 2013 and Estes et al., 2011). This model can be used to validate candidate anti-diabetic compounds identified in the primary screen) and to establish which components of the insulin signaling pathway can be critical for developing therapeutic strategies.

### d. TDP-43 Protein is a Component of Ubiquitin Positive Cytoplasmic Inclusions Found in ALS Patients

A hallmark of neurodegenerative disorders is the presence of pathological cytoplasmic inclusions within neurons or the surrounding glia. These inclusions are sites of protein aggregation that often times contain ubiquitin and in some cases have been shown to have a protective role while in others have been shown to be toxic. In 2006, Neumann and colleagues (3) reported the identification of TDP-43 protein in ubiquitin positive inclusions from ALS and a subset of FTLD patients. Since then, extensive pathological studies have identified TDP-43 as a common component of cytoplasmic inclusions found in all but familial SOD1 cases studied to date (4, 5) as well as other neurodegenerative disorders (1). Histological examinations of human tissue obtained at autopsy have defined distinct sub-types of TDP-43 positive cytoplasmic inclusions ranging in shape from filamentous to round aggregates that are present in neurons and sometimes in the surrounding glia (3, 6).

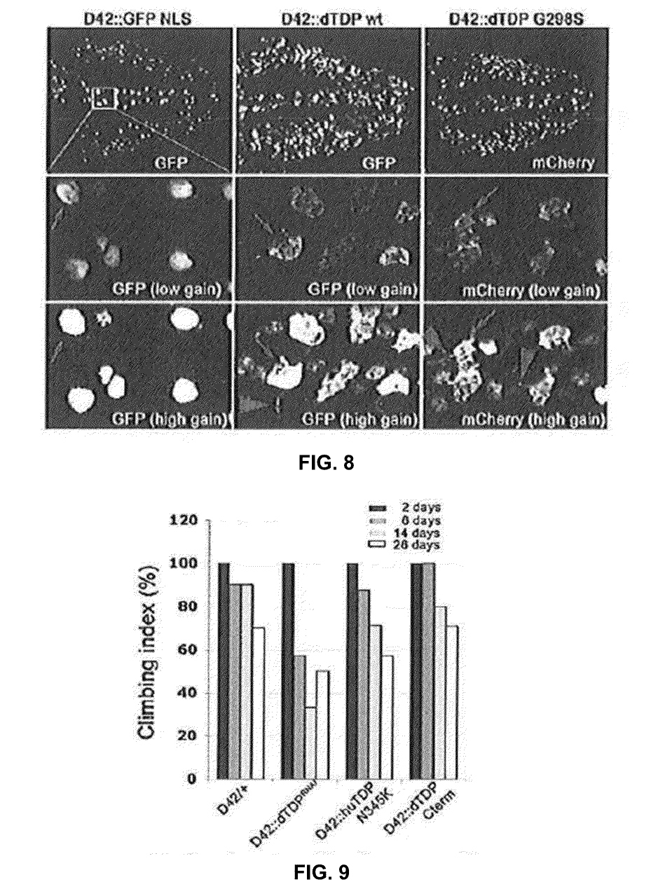

The formation of TDP-43 cytoplasmic inclusions has been correlated with a change in the subcellular distribution of TDP-43, which is normally nuclear (3, 7, and 8) (FIGS. 8 and 9). The density of cytoplasmic inclusions correlates with the number of TDP-43 positive bodies, indicating a direct relationship between TDP-43 and the pathology of ALS. Taken together, these data indicate that ALS is a TDP-43 proteinopathy, thus making TDP-43 a candidate for genetic screening and a target for therapeutic approaches.

### e. Mutations in TDP-43 can Cause ALS by Accelerating Protein Aggregation

In 2008, an avalanche of articles reported the identification of TDP-43 gene mutations in both familial and sporadic ALS patients of diverse ethnicities (9-15). Except for a single mutation found in the first RNA binding domain of TDP-43 (RRM1, FIG. 1), all other mutations found in ALS patients lie in the C-terminus, including the Glycine rich domain (FIG. 1). These data indicate that TDP-43 is not just another consequence of the disease but a causative agent with the potential of providing valuable insights into the pathology of neurodegeneration.

For example, FIG. 1 shows a schematic representation of Drosophila (dTDP-43) and human TDP-43 (huTDP-43). Homologs of TDP-43 have been identified in animal models including Drosophila, C. elegans. Structurally, the fly, worm and human proteins are comprised of similar domains (FIG. 1) and exhibit comparable specificity in binding UG repeats. However, only the Drosophila homolog (dTPD-43) has the ability to inhibit splicing in a manner similar to human TDP-43. Local pair-wise alignment indicates 38% amino acid identity between the worm and human proteins and 59% between the fly and its human counterpart (22). The structural and functional conservation between human and fly TDP-43 as well as the data described herein serve as the basis for the establishment of a Drosophila ALS model. In FIG. 1, NES means Nuclear Export Signal, RRM1 and RRM2 mean RNA Recognition Motifs 1 and 2, and G-rich means Glycine rich domain (dark green).

Recent studies using in vitro assays and a yeast model of TDP-43 proteinopathy showed that TDP-43 protein is intrinsically prone to aggregation (16). Structure-function studies demonstrated that the C-terminus domain of TDP-43 is critical for aggregation in vitro and is toxic in vivo. The introduction of ALS-linked TDP-43 mutations in yeast led to the formation of cytoplasmic foci that led to cell death. Furthermore, in vitro studies showed that the presence of C-terminus mutations led to an accelerated aggregation of the wild-type protein, which itself can form aggregates that resemble those found in ALS (16). A recent study reported that mutant TDP-43 accumulates in cytoplasmic foci in primary motor neuron cultures and in a zebrafish model (17). In contrast, expression of mutant TDP-43 in the mouse nervous system led to the accumulation of ubiquitin positive foci in motor neurons but without the aggregation of TDP-43 itself (8). Taken together, these studies demonstrate that mutations in the C-terminus domain of TDP-43 plays a critical role in the pathogenesis of ALS and animal and cellular models for the mutations found in human patients are likely to provide important insights into the mechanisms of disease.

### f. Identification of Novel Therapeutic Targets for ALS Through Genetic and Drug Screens in Drosophila

To discover novel TDP-43 interacting genes as well as potential therapeutic compounds for ALS genetic and drug screens can be performed based on two already identified phenotypes: (i) the adult locomotor defects and (ii) the adult eye neurodegeneration. All newly identified genes and compounds of interest can be validated through secondary tests including the presence of cytoplasmic inclusions, neuromuscular junction and any additional phenotypes discovered as a result of expressing mutant TDP-43.

First, the genetic screen can be performed. The Drosophila deficiency kit can be crossed with the TDP-43 mutants and tested for the dominant suppression or enhancement of the phenotypes described. As genomic regions of interest are identified, the resolution of the screen can be increased by testing smaller deletions and available point mutations until the individual loci that interact with TDP-43 are identified. With this approach, new genes can be identified that interact with TDP-43 during normal neuronal development and/or can be involved in the etiology of ALS.

Second, the drug screens can be performed. The Spectrum drug collection, which consists of 2,000 of which 50% are FDA-approved drugs, can be administered to flies expressing mutant TDP-43 in motor neurons and the ability of individual compounds to rescue the locomotor phenotypes can be tested. The Preswtick drug collection, which consists of 1,200 FDA-approved drugs, can be administered to flies expressing mutant TDP-43 in motor neurons and the ability of individual compounds to rescue the locomotor phenotypes can be tested. This approach can identify compounds that rescue TDP-43 phenotypes and thus offer potential therapies for ALS that can be rapidly moved into clinical trials.

### g. Loss of dTDP-43 Function LED to Defects in the in Larval Neuromuscular Junction Architecture

To develop a Drosophila model of ALS based on TDP-43, the effect of dTDP-43 mutations on the architecture of the larval neuromuscular junction (NMJ) were tested (29). Two P-element lines, P(SUPor-P)TBPH^{KG08578} and P(EPgy2)TBPH^{EY0530}, which are inserted in the dTDP-43 locus (FIG. 2) and do not affect the viability of the fly were obtained. These alleles are hypomorphic, as recently reported (6). A chromosomal deletion, Df(2R)or-BR6, which spans several kb of genomic DNA (estimated cytology: 59D5-59D10; 60B3-60B8) and encompasses several loci including dTDP-43 was also obtained. In addition, two different RNAi lines targeting the dTDP-43 open reading frame were obtained (FIG. 2). These RNAi constructs are under the control of the UAS promoter and thus can be used to knock-down dTDP-43 in motor neurons specifically, in contrast to the loss of function mutants, which affect the expression of dTDP-43 throughout the whole animal.

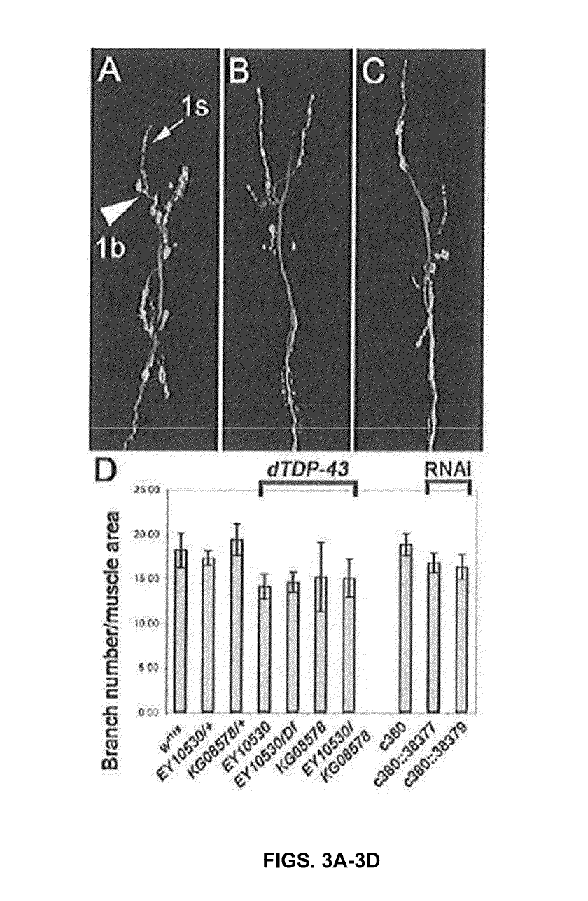

For example, FIG. 2 shows a schematic representation of the dTDP-43 gene located on the second chromosome. dTDP-43 gene structure, P-element insertions, predicted isoforms, RNAi target sequence (GD6943) and Df(2R)or-BR6 were as indicated. Next, the morphology of larval NMJs in different allelic combinations, including TBPH^{EY10530} and TBPH^{KG08578} homozygotes, TBPH^{EY0530}/Df(2R)or-BR6, TBPH^{EY10530}/TBPH^{KG08578} and RNAi knockdown in motor neurons (c380:: TBPH RNAi) compared to several controls, were examined. To determine whether dTDP-43 was required for the development of the larval NMJ, the total number of synaptic boutons as labeled by the presynaptic marker CSP (30), and the number of axonal branches were measured. The data indicate that loss of dTDP-43 had no effect on synaptic boutons but reduced axonal branching (FIG. 3). Feiguin et al. reported similar albeit more severe phenotypes with two null alleles, Δ23 and Δ142 (6). In addition, the nulls exhibited larval locomotor defects with spastic, uncoordinated movements (6). These phenotypes were rescued by either dTDP-43 or huTDP-43 expression in the null dTDP-43 mutant background (6). All of this data demonstrates that dTDP-43 was required for the proper development and function of the larval NMJs and that dTDP-43 and its human counterpart were functionally equivalent in the fly. Similar phenotypic studies can be conducted to determine the consequences of mutant TDP-43 expression at the NMJ.

FIG. 3 shows a comparative analysis of dTDP-43 and wild-type larval NMJs. (A) wild-type NMJ (w¹¹¹⁸) labeled with the synaptic vesicle marker CSP and the membrane marker HRP. Type 1b (arrowhead) and is (arrow) synaptic boutons. (B, C) Representative images of dTDP-43 mutant NMJs showed different degrees of reduced complexity compared to wild-type. (B) TBPH^{EY10530}/Df(2R)or-BR6 (EY10530/Df). (C) TBPH^{EY10530} homozygotes (EY10530). (D) Loss of dTDP-43 resulted in significantly less complex NMJs (Pᵥₐₗᵤₑ<0.05). Genotypes as indicated. dTDP-43 bracket indicates loss of function alleles; RNAi bracket indicates RNAi lines driven by the motor neuron Gal4 driver c380 (31).

### h. dTDP-43 was Required for Normal Climbing Behavior in Adults

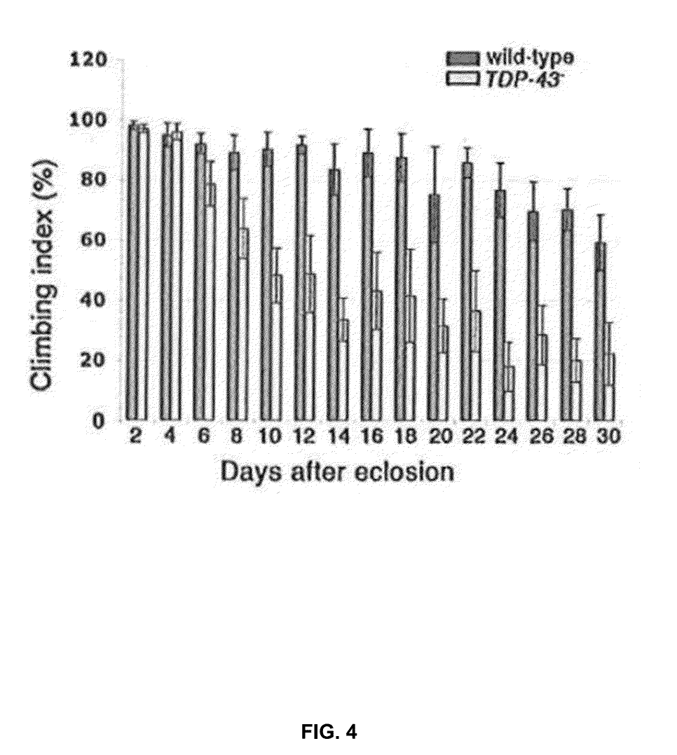

To further investigate the requirement of dTDP-43 in motor function a negative geotaxis climbing assay (32) was used that has been used to successfully assess motor neuron function in fly models of Parkinson's disease (33) and hereditary spastic paraplegia (34). As shown in FIG. 4, initially, dTDP-43 loss of function mutants showed a climbing ability comparable to that of age-matched wild-type control flies but approximately six days post-eclosion, they exhibited a decline. This continued to exacerbate during the thirty day testing period, when only 20% of mutant flies pass the climbing test compared to more than 60% wild-type flies. Similar results were obtained when TDP-43 was knocked-down by RNAi in motor neurons using the D42 Gal4 driver (35) (FIG. 9). These data indicate that the motor neuron dysfunction found in ALS patients is a manifestation of TDP-43 loss of function in neurons. This issue can be further addressed when various forms of TDP-43 are expressed in both wild-type and dTDP-43 mutants.

For example, FIG. 4 shows that dTDP-43 was required for adult climbing behavior. The climbing index of dTDP-43 mutant adults (P(SUPor)TBPH^{KG08578}) was significantly altered during the 30 day trial period. These data represent averages of two independent trials in which >100 males were used per genotype. Similar results were obtained for the P(EPgy2)TBPH^{EY10530} allele.

### i. Wild-Type and Mutant TDP-43 Expression in the Eye Neuroepithelium was Toxic

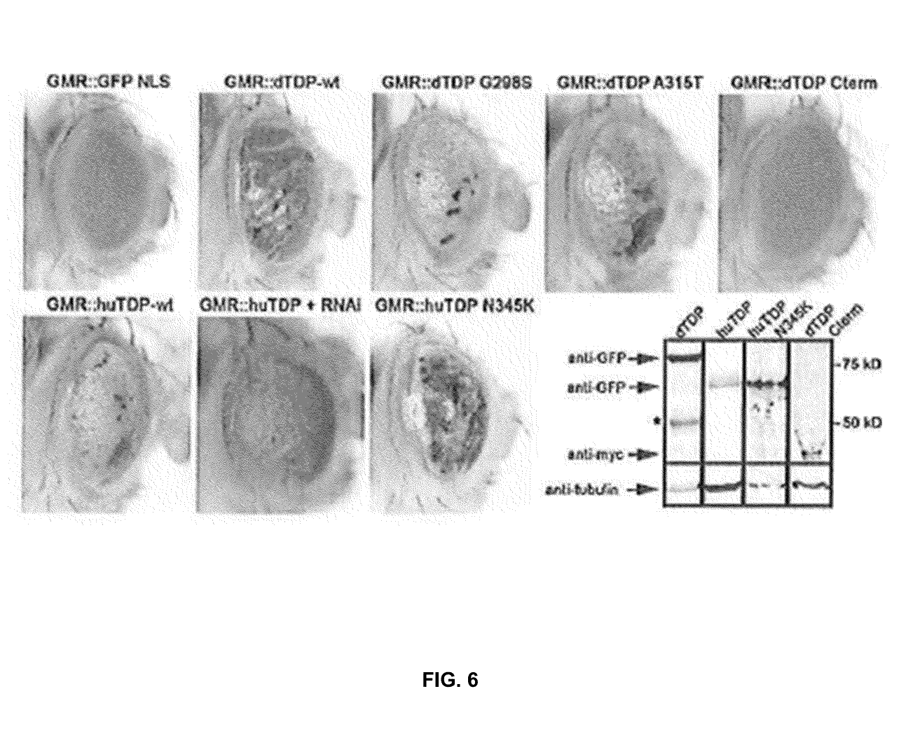

Loss of function mutations in dTDP-43 had no obvious effect on the morphology of the adult fly, including the eye (6). To determine whether TDP-43 was toxic wild-type and various forms of TDP-43 were expressed in the eye, which consists of hundreds of photoreceptor neurons as well as accessory cells and has been successfully used to model human neurodegenerative disorders (25, 38). In FIG. 6, wild-type and mutant TDP-43 expression resulted in a visible rough eye phenotype with loss of pigmentation, which was indicative of retinal neurodegeneration and cell loss. The expression of the C-terminus had no phenotype, indicating that this fragment alone was not toxic and thus its presence in ALS inclusions can simply be a consequence and not a cause of neuronal toxicity. The co-expression of huTDP-43 and TDP-43 RNAi alleviated the toxicity of huTDP-43 expressed alone. This was due to a reduction in the level of endogenous dTDP-43 (compare GMR::huTDP-43-wt to GMR::huTDP+RNAi in FIG. 6). Similar rescue experiments can be performed for all transgenes. Similar results were obtained with dTDP-43 RNAi, indicating that these toxic phenotypes mimic TDP-43 loss of function. Plastic sections can be used to confirm the presence of neurodegeneration in the retina.

For example, FIG. 6 shows wild-type and various mutant forms of fly and huTDP-43 result in rough eyes accompanied by loss of pigmentation and necrosis. Genotypes as indicated. Bottom right panel: Western blot probed for GFP and myc tags (top) shows various transgenes running at their predicted sizes: 85 kD for GFP-dTDP, 72 kD for huTDP-YFP and 33 kD for myc(6×)-dTDP Cterm. Asterisk indicates a smaller product of GFP-dTDP, possibly due to degradation. Tubulin was used as a loading control (bottom).

### j. Wild-Type and Mutant TDP-43 Aggregated in the Eye Neuroepithelium and Motor Neurons

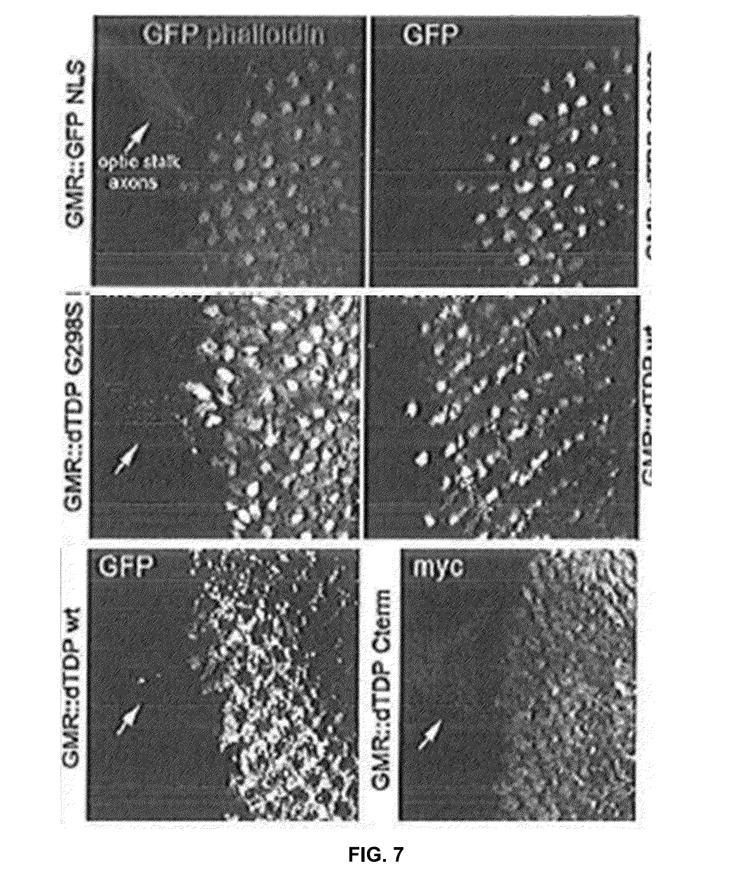

The data (FIGS. 3 and 6) indicate a loss of function mechanism for the TDP-43 neurotoxicity. The subcellular localization of wild-type and mutant TDP-43 was examined in photoreceptor (FIG. 7) and motor neurons (FIG. 8). FIG. 7 shows that wild-type and mutant TDP-43 aggregated in photoreceptor neurons. Larval eye imaginal discs showing the localization of various forms of TDP-43 as marked by GFP, mCherry or myc tags. The abnormal localization and the presence cytoplasmic (red arrows) and axonal aggregates (white arrows) due to dTDP G298S and wild-type dTDP expression (compare to GFP NLS control). Genotypes as indicated. Images shown are single confocal slices or 2-3 slice projections. Phalloidin marks cell shapes and axons. GFP and mCherry were imaged following a short fixation. Myc marks the C terminus domain, which appeared entirely cytoplasmic (due to the lack of the NLS).

As shown in FIG. 7, wild-type and mutant TDP-43 but not the C terminus accumulated in inclusions both in the cell body and in axons. This correlates with the phenotypes shown in FIG. 6 and indicates that TDP-43 toxicity was due to its aberrant cytoplasmic aggregation. Similarly, in motor neurons, wild-type and mutant TDP-43 accumulate in cytoplasmic aggregates (FIG. 8).

FIG. 8 shows that wild-type and mutant TDP-43 aggregated in motor neurons. Ventral ganglia (top panels) showing the localization of various forms of TDP-43 as marked by GFP or mCherry tags. Bottom panels show high magnification view of midline motor neurons. Two different gains were used to show the abnormal perinuclear and cytoplasmic localization of wild-type dTDP and dTDP G298S in comparison to the GFP NLS control (red arrows). GFP and mCherry were imaged after a short fixation. Genotypes as indicated.

Taken together, these data indicate that TDP-43 toxicity was linked to its abnormal accumulation in cytoplasmic aggregates and a partial depletion from the nucleus. The connection between TDP-43's abnormal subcellular localization and its toxicity can be tested using genetic rescue experiments. Specifically, whether reduction in endogenous TDP-43 rescues the overexpression of wild-type but not mutant TDP-43 can be tested and can determine how this correlates with changes in TDP-43's subcellular localization.

### k. Characterization of Adult Locomotor Defects

Proposed experiments can determine whether the lethality is due to motor neuron death induced by TDP-43 aggregation. The lethality issue can be overcome by using lower expressing transgenics and inducible Gal4 drivers (39). Here the climbing behavior of dTDP RNAi, HuTDP N345K and dTDP Cterm expression was tested in motor neurons using the D42 driver (35). These data show that dTDP RNAi and HuTDP N345K but not dTDP C term resulted in a reduced climbing index (FIG. 9).

FIG. 9 shows mutant TDP-43 overexpression and TDP-43 RNAi but not the C terminus of dTDP-43 affected adult locomotor behavior. Climbing index measured as described (FIG. 4). Genotypes and testing days as shown. Ten flies were used per genotype.

### l. Feasibility of Genetic Screens

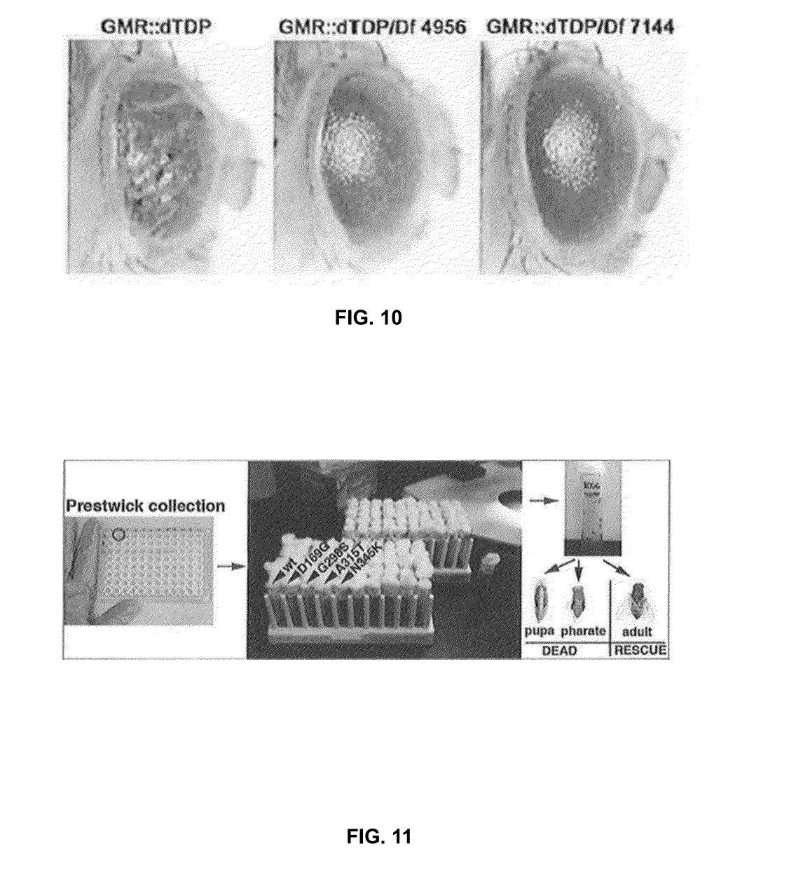

To test the feasibility of the genetic screens, a pilot screen was performed based on the rough eye phenotype caused by dTDP RNAi expression in the eye. Forty (40) second chromosome deficiencies were tested for their ability to dominantly modify the dTDP RNAi rough eye and found 4 suppressors and one enhancer. Next, whether these candidate deficiencies also modify the more pronounced phenotype due to wild-type dTDP overexpression in the eye was tested (FIG. 6). As shown in FIG. 10, two of these deficiencies, namely Df 4956 and 7144 were strong suppressors of the dTDP overexpression phenotype.

FIG. 10 shows that a dominant modifier genetic screen in the eye identifies two candidate genomic regions as TDP-43 interactors. Genotypes as indicated. Suppression of necrosis and roughness by Df 4956 and 7144 occurred. Some loss of pigmentation remained.

### m. Summary of Results of Example 1

A Drosophila model of ALS based on TDP-43 has been established. The Drosophila genome harbors a single TDP-43 homolog, namely dTDP-43 that is 59% identical in sequence to its human counterpart. Phenotypic analyses using three different mutant alleles show that loss of function for dTDP-43 results in morphological defects at the larval neuromuscular junction and motor dysfunction in the adult thus indicating that dTDP-43 was required for the proper development and function of motor neurons. These results were supported by RNAi experiments in which dTDP-43 was knocked-down specifically in motor neurons using the bipartite Gal4-UAS system (37). Additionally, Feiguin et al. (6) showed that both Drosophila and human TDP-43 expression in motor neurons can rescue the loss of function phenotypes in the fly model. Taken together, the results show that dTDP-43 was functionally conserved and was required for proper morphology and function in developing motor neurons. Data has been obtained indicating that wild-type and mutant TDP-43 formed cytoplasmic aggregates that correlate with neuronal toxicity in the eye and lethality in motor neurons. At least one mutant form of TDP-43 (hu N345K) resulted in reduced locomotor activity when expressed specifically in motor neurons.

### 2. Experimental Design for Example 1

### a. Establishment a TDP-43 Based Drosophila Model of ALS

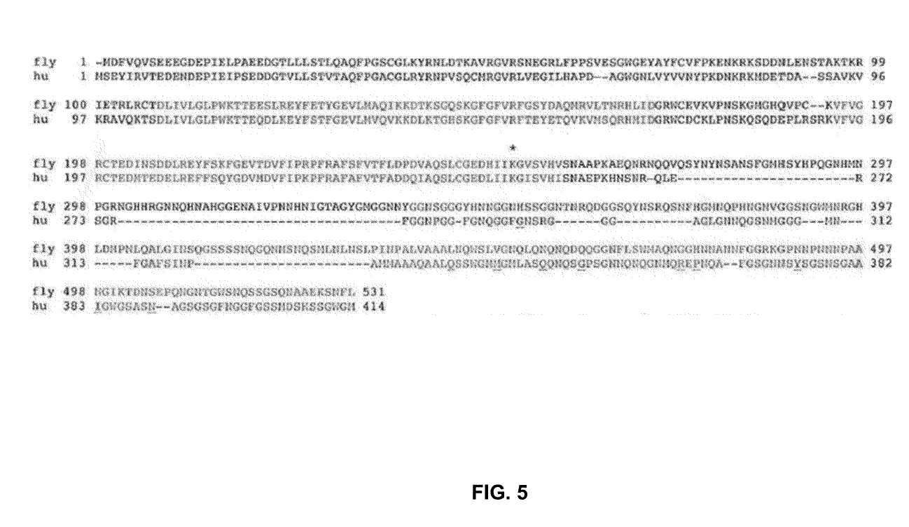

A “reverse translational” approach can be used to model ALS. To this end, constructs and subsequently, transgenic lines that express both wild-type and mutant forms of TDP-43, which mimic the mutations found in human patients were generated. Upon aligning the fly and human proteins (FIG. 5, adapted from ref 22), eight residues mutated in human patients that are conserved in the fly were identified. Of these, D169G lies within the RRM1 domain, while the other seven residues are located within the more divergent C-terminus domain of the protein. The remaining ten residues with corresponding mutations in human patients and not conserved in the fly, lie within the C-terminal domain.

For example, FIG. 5 shows an amino acid sequence comparison between Drosophila (fly) and human (hu) TDP-43. RNA Recognition domains (RRM1 and RRM2) shown in blue font. C-terminus domain shown in green font (darker green corresponds to the Glycine-rich domain within the C-terminus). Amino acids mutated in human patients and conserved between the fly and human proteins are shown in red. Underlined residues correspond to human mutations in non-conserved amino acids. Asterisk marks the beginning of the C-terminus fragment that has been identified within cytoplasmic inclusions.

While all known mutant forms of TDP-43 can provide insights into the mechanisms of the disease and can be generated, for practical reasons, those mutations that are conserved between fly and human and/or their toxicity has been established in yeast (16), D169G, G298S, A315T and N345K, were initially used. In addition, transgenics that express the C-terminal fragment linked to the pathology of a wide spectrum of ALS cases can be generated (36). To directly compare the toxicity of the fly and human proteins in vivo, both the fly and human proteins were manipulated. All transgenes can be expressed in a tissue specific manner using the bipartite Gal4-UAS system (37). To eliminate positional effects due to random insertion in the genome, multiple stocks for each transgene are established. These tools can be used to establish the full range of TDP-43 phenotypes in neurons.

The results indicate that loss of dTDP-43 recapitulated some of the neuromuscular and locomotor defects associated with ALS. To further establish a Drosophila model several transgenic lines expressing wild-type as well as mutant forms of TDP-43 that mimic the mutations found in human patients have been generated. Data using these new tools indicated that both wild-type and mutant TDP-43 expression in motor neurons and the eye neuroepithelium leads to cellular and organismal phenotypes that resembled the ALS pathology.

The transgenic lines (on average 10/construct) have been mapped and tested for expression and phenotypes as shown in the Results section. Next, whether the wild-type dTDP-43 and huTDP-43 (wild-type TDP-43) can rescue the dTDP-43 loss of function phenotypes can be tested (FIGS. 3 and 4). Experiments in the eye (FIG. 6) indicate that dTDP RNAi alleviates the toxicity of wild-type huTDP-43, likely by reducing endogenous dTDP-43 levels. The constructs are tagged with GFP (and/or mRFP), thus can ensure that the transgenically provided proteins are properly localized. For these experiments, the Gal4 driver D42 can be used to express wild-type TDP-43 in motor neurons into a dTDP-43 mutant background. The rescuing ability of the wild-type transgenes can be determined by comparison of the transgenics' larval NMJ morphology and climbing behavior to that of the dTDP-43 mutants (loss of function and RNAi).

### b. does Mutant TDP-43 Form Cytoplasmic Aggregates In Vivo?

ALS is linked to the mis-localization and aggregation of mutant or truncated TDP-43 into ubiquitin-positive cytoplasmic inclusions (9). To test whether this occurs in the model, mutant dTDP-43 and huTDP-43 as well as C-terminus TDP-43 can be expressed in motor neurons, then use immunohistochemistry to determine whether these aberrant TDP-43 forms aggregate in the neuronal cytoplasm as they do in yeast (16). The data showed that this is indeed the case (FIGS. 7 and 8). Whether ubiquitin-positive inclusions are present in the cytoplasm can also be tested and whether TDP-43 aggregates co-localize with ubiquitin as found in patient tissue samples can be tested. All mutant and truncated TDP-43 can be expressed in both wild-type and mutant dTDP-43 backgrounds. These tests can be performed with adults aged 0, 10, 20 and 30 days post-eclosion. Tests for the presence of ubiquitin-positive inclusions in the thoracic ganglia of RNAi knock-down adults were preformed, which exhibit locomotor defects but could not detect any. These results indicated that the cytoplasmic inclusions found in ALS patients are not a simple indicator of motor neuron dysfunction but rather a symptom of TDP-43 misfolding and/or misregulation. In addition, these data predicted that the expression of mutant and/or truncated TDP-43 was needed for the formation of cytoplasmic inclusions as recently found in yeast (16) and shown by the data (FIGS. 7 and 8). These experiments can be used in determining the causality between the expression of mutant TDP-43, the formation of cytoplasmic inclusions and the presence of ubiquitin and can firmly establish the extent to which this fly model recapitulates the human pathology.

To further evaluate the similarity to the inclusions present in human patients, the mutant TDP-43 expressing tissues can be subjected to velocity sedimentation (27). Briefly, whole males expressing mutant TDP-43 under the control of the panneuronal elavGal4 driver, can be homogenized in high salt buffer (750 mM NaCl, 50 mM Tris, 10 mM NaF, 5 mM EDTA), then subjected to 100,000 g centrifugation. The supernatant and the resuspended pellet can be analyzed by Western blot to determine the distribution of mutant TDP-43 between the soluble and insoluble fractions. These biochemical assays can determine the solubility of the different mutant forms of TDP-43 and can be performed in conjunction with climbing assays. Taken together, these experiments can determine the correlation between the cellular/molecular and functional phenotypes in this fly model. Importantly, these assays can serve as validation tests for any gene and/or compound identified.

### c. Test for Locomotor Defects Due to Expression of Mutant TDP-43 in Motor Neurons

To determine the functional consequences of mutant TDP-43 expression in motor neurons the different mutant transgenes can be expressed under the control of the D42 driver and perform climbing assays. Flies display a negative geotaxis response when given a mechanical stimulus (32). For the duration of the testing period, flies can be maintained on standard medium with fresh yeast that is changed every two to three days. Flies can be collected 0-8 hours after eclosion and can be separated by sex. 100 flies can be used for each genotype (10 flies each in 10 vials, 50 males and 50 females). All flies can be tested for their ability to perform this assay every two days. Each group of 10 flies can be placed within an empty vial with a circular mark demarcating 5 cm from the bottom of the vial. These flies can then be gently tapped to the bottom of the vial, and given 18 seconds to orient themselves and climb towards the top. After 18 seconds, the number of flies that have successfully performed this assay can be scored and recorded. Before each test is performed, the number of viable flies within each vial can be counted, and any dead flies can be discarded and no longer considered for the assay. The results can then plotted over time (30 days), and any defects in climbing ability can be compared against the appropriate control flies (FIGS. 4, 9). Paired student's t-tests can be performed to determine any statistically significant differences between mutant flies and controls. These assays can be used as validation tests for the screening assays.

### d. Regulation of Synaptic Transmission in the Giant Fiber Circuit

Muscle weakness is accompanied by reduced motor nerve conductance in a number of motor neuron diseases. To determine whether this also occurs in the fly model, defects in synaptic transmission due to alterations in TDP-43 (loss of function, RNAi knock-down and expression of mutant TDP-43) can be measured. The giant fiber (GF) system, a well-established motor circuit in Drosophila, which controls jump-flight escape behavior can be used (40). Importantly, the D42 Gal4 line used in the studies drives expression in several motor neurons including the components of the giant fiber system (27). The GF neuron (GFn) is located in the brain and descends to the thoracic ganglia where it excites the motor neuron TTM. GFn also excites the peripherally synapsing interneuron (PSI), which in turn excites five motor neurons, DLM. GFn activation and intracellular recordings from TTM muscles can be obtained from adult flies in a method similar to that described previously (40-42). Briefly, flies can be anesthetized by cooling down on ice and waxed, ventral side-down, onto a small podium in a Petri dish. The wings can be waxed down in an outward position. The GFs can be activated extracellularly with brain stimulation by two etched tungsten electrodes, one placed through each eye into the supraoesophageal ganglion. A pulse of ˜10-20 V for 0.03 msec from an AM Systems stimulator is sufficient to give the short latency associated with direct excitation. Slightly larger amplitude stimuli can be used to ensure that threshold is always exceeded. A tungsten electrode placed in the abdominal cavity serves as a ground. Saline-filled glass electrodes pulled to a resistance of 40-60 MΩ will be used to record intracellulary from the muscle fibers. Recordings can be amplified using an Axoclamp 2B amplifier and stored using pClamp software with a Digidata A/D interface (Molecular Devices). Each animal can be subjected to two standard tests: response latency and following frequency. For latencies, each fly will be given 10 single pulses. Measurements can be taken from the beginning of the stimulation artifact to the beginning of the EPSP. For following frequency, each animal can be given 10 pulses at 100 Hz. These experiments can determine whether alterations in TDP-43 using either loss of function mutations, RNAi knock-down or expression of mutant TDP-43 result in diminished motor conductance and thus resemble the features of human motor disease. In addition, this approach can be used as a validation assay for any gene or drug found.

### e. Test for Eye Phenotypes

To determine whether mutant TDP-43 also causes defects in the architecture of the neuroepithelium, the expression of the appropriate transgenes can be driven using GMR Gal4. Because the eye is sensitive to imprecise development, if any of these conditions causes even slight changes in cellular development the result can be a rough eye. Plastic sections can be used to determine the effects on eye morphology and immunohistochemistry experiments can establish the presence of cytoplasmic inclusions and/or neuronal death. These phenotypes can be used as validation tests for the screens.

### f. Identification of Novel Therapeutic Targets for ALS Through Genetic and Drug Screens in Drosophila

RNAi knock-down of dTDP-43 in motor neurons leads to locomotor defects and morphological abnormalities. In addition, wild-type and mutant TDP-43 expression leads to visible rough eyes accompanied by necrosis as well as locomotor defects. These two phenotypes can be used as the basis for genetic and drug screens with the goal of discovering novel TDP-43 interacting genes as well as potential therapeutic compounds for ALS. These phenotypes have been chosen because: (i) they arise from TDP-43 manipulation (loss or overexpression) in a subset of specific neurons, (ii) they are robust, (iii) they are easy to score and (iv) they are amenable to F1 screens. All newly identified genes and compounds of interest can be validated through secondary tests including the presence of cytoplasmic inclusions, neuromuscular physiology and any additional phenotypes that have already been established (FIGS. 3, 4, and 6-9) or are discovered as a result of expressing various forms of mutant TDP-43. This approach provides a good position for identifying genes that interact with TDP-43 during normal neuronal development and/or can be involved in the etiology of ALS. Furthermore, data obtained indicate the feasibility of the proposed approach by identifying two candidate genomic regions that interact with TDP-43 (FIG. 10).

### g. Genetic Screens

For the climbing behavior screen the following stock was built: w¹¹¹⁸; UAS TBPH RNAi 38379; D42 Gal4:: UAS TBPH RNAi 38377 (D42::double RNAi). For the rough eye screens, w¹¹¹⁸; GMR Gal4::UAS TBPH RNAi 38379; UAS TBPH RNAi 38377 (GMR Gal4::double RNAi) was built and a stock based on wild-type dTDP-43 overexpression is being built (FIG. 10). The Drosophila deficiency kit can be crossed with the climbing behavior and rough eye screen stocks individually. There are a total of 222 deletion stocks on the second and third chromosomes combined (110 and 112, respectively) which are estimated to cover 93% of euchromatin of the approximately 80% genome contained on these autosomes. In the F1 generation, whether individual deficiencies can modify (suppress or enhance) the eye phenotypes due to the expression of dTDP-43 in the eye can be tested. Since the eye screens are less labor intensive the screens using the eye phenotype can be used initially (FIG. 10). The candidate deficiencies can be validated based on their ability to also modify the climbing defect of D42::double RNAi adults at 0, 10, 20 and 30 days post eclosion. These assays can be performed as described herein. These flies can easily be obtained from a few small-scale crosses with 6-10 parents each. This first round of screening can be followed by a second confirmatory trial and validation tests. These candidate deficiencies can be validated by testing for their ability to suppress the phenotypes due to mutant TDP-43 expression, such as the presence of ubiquitin positive cytoplasmic inclusions, functional defects in the giant fiber motor circuit and anatomical defects at the neuromuscular junction. As genomic regions of interest are identified, coarse then fine scale genetic mapping can be performed by testing smaller deletions and available P-element insertions and/or point mutations until the individual loci that interact with TDP-43 is identified.

### h. Drug Screens

Pharmacological intervention can rescue the morphological and functional defects due to altered TDP-43 in neurons. The Spectrum drug collection (MicroSource Discovery Systems, Inc.), which includes 50% FDA approved drugs can be used as well as natural compounds. The Prestwick drug collection, which includes 1200 FDA approved drugs can be used as well.

In the first phase of the drug screen, the Spectrum collection or the Prestwick collection can be fed to Drosophila larvae that express dTDP-43 RNAi in motor neurons (D42::double RNAi) and can test for the ability of individual compounds to rescue the locomotor and neurodegeneration phenotypes, respectively. 12 homozygous embryos of the appropriate genotype can be collected and placed in 96 well plates containing standard yeast-cornmeal food supplemented with the Spectrum compounds at 40 μM, then allowed to develop to the pupal stage. These conditions have been successful previously, in a drug screen for compounds that rescue Fragile X phenotypes in Drosophila (44). Next, pupae can be carefully transferred to regular food vials containing yeast paste to which individual drugs have also been supplemented to 40 μM. Eclosing adults can be collected and tested using the climbing assays as described.

In the second phase of the drug screen, those compounds that rescue the climbing defect by at least 20% in phase 1, can be retested in duplicate at 10 μM, 20 μM, 40 μM, 50 μM and 60 μM to determine the optimum drug concentration, which can be based on the following criteria: (i) it should have the maximum “rescuing” effect on climbing defect in D42::double RNAi flies and (ii) it should have no effect on control flies (D42 driver alone). Thus only those compounds that have a specific effect on TDP-43 phenotypes but do not affect normal motor neuron development and function are selected for further testing.

In the second phase of the drug screen, triplicate testing of the drug concentration determined in phase 2 to be optimal for rescuing the TDP-43 motor neuron phenotypes can be performed. At this stage the candidate compounds can be tested in adults to determine their ability to rescue motor dysfunction without concerns for developmental effects.

During drug screen validations, the therapeutic potential of the candidate compounds can be confirmed by testing for their ability to suppress the phenotypes due to mutant TDP-43 expression, including the presence of cytoplasmic aggregates, functional defects in the giant fiber motor circuit and anatomical defects at the neuromuscular junction. These experiments can confirm the rescue efficacy and determine the statistical significance of the rescuing effect for any compound deemed to be of interest. Furthermore, the feasibility for adult stage intervention can be established and thus identify potential therapies for ALS that can be rapidly moved into clinical trials.

A Drosophila ALS model using both loss and gain of function paradigms can be established. The critical feature of this model is the use of TDP-43, which has been identified as a major component in most ALS-specific cytoplasmic inclusions. Through the use of genetic screens genes that interact with TDP-43 in neurons can be identified and can be unknown ALS loci. In addition, the pharmacological approach is likely to uncover therapeutic agents that will target the TDP-43 linked motor dysfunction, possibly by targeting cytoplasmic aggregates, a common denominator for most ALS cases known to date.

### i. References for Example 1

1. Banks, G. T., et al. (2008) TDP-43 is a culprit in human neurodegeneration, and not just an innocent bystander. Mamm Genome, 19, 299-305.
2. Beleza-Meireles, A. et al. (2009) Genetic studies of amyotrophic lateral sclerosis: controversies and perspectives. Amyotroph Lateral Scler, 10, 1-14.
3. Neumann, M., et al. (2006) Ubiquitinated TDP-43 in frontotemporal lobar degeneration and amyotrophic lateral sclerosis. Science, 314, 130-3.
4. Maekawa, S., et al. (2009) TDP-43 is consistently co-localized with ubiquitinated inclusions in sporadic and Guam amyotrophic lateral sclerosis but not in familial amyotrophic lateral sclerosis with and without SOD1 mutations. Neuropathology.
5. Tan, C. F., et al. (2007) TDP-43 immunoreactivity in neuronal inclusions in familial amyotrophic lateral sclerosis with or without SOD1 gene mutation. Acta Neuropathol, 113, 535-42.
6. Feiguin, F., et al. (2009) Depletion of TDP-43 affects Drosophila motoneurons terminal synapsis and locomotive behavior. FEBS Lett, 583, 1586-92.
7. Arai, T., et al. (2006) TDP-43 is a component of ubiquitin-positive tau-negative inclusions in frontotemporal lobar degeneration and amyotrophic lateral sclerosis. Biochem Biophys Res Commun, 351, 602-11.
8. Wegorzewska, I., et al. (2009) TDP-43 mutant transgenic mice develop features of ALS and frontotemporal lobar degeneration. Proc Natl Acad Sci USA, 106, 18809-14.
9. Daoud, H., et al. (2009) Contribution of TARDBP mutations to sporadic amyotrophic lateral sclerosis. J Med Genet, 46, 112-4.
10. Gitcho, M. A., et al. (2008) TDP-43 A315T mutation in familial motor neuron disease. Ann Neurol, 63, 535-8.
11. Kabashi, E., et al. (2008) TARDBP mutations in individuals with sporadic and familial amyotrophic lateral sclerosis. Nat Genet, 40, 572-4.
12. Kuhnlein, P., et al. (2008) Two German kindreds with familial amyotrophic lateral sclerosis due to TARDBP mutations. Arch Neurol, 65, 1185-9.
13. Pamphlett, R., et al. (2009) TDP-43 neuropathology is similar in sporadic amyotrophic lateral sclerosis with or without TDP-43 mutations. Neuropathol Appl Neurobiol, 35, 222-5.
14. Rutherford, N. J., et al. (2008) Novel mutations in TARDBP (TDP-43) in patients with familial amyotrophic lateral sclerosis. PLoS Genet, 4, e1000193.
15. Sreedharan, J., et al. (2008) TDP-43 mutations in familial and sporadic amyotrophic lateral sclerosis. Science, 319, 1668-72.
16. Johnson, B. S., et al. (2009) TDP-43 is intrinsically aggregation-prone and ALS-linked mutations accelerate aggregation and increase toxicity. J Biol Chem.
17. Kabashi, E., et al. (2009) Gain and loss of function of ALS-related mutations of TARDBP (TDP-43) cause motor deficits in vivo. Hum Mol Genet.
18. Ou, S. H., et al. (1995) Cloning and characterization of a novel cellular protein, TDP-43, that binds to human immunodeficiency virus type 1 TAR DNA sequence motifs. J Virol, 69, 3584-96.
19. Wang, I. F., et al. (2008) TDP-43, the signature protein of FTLD-U, is a neuronal activity-responsive factor. J Neurochem, 105, 797-806.
20. Johnson, B. S., et al. (2008) A yeast TDP-43 proteinopathy model: Exploring the molecular determinants of TDP-43 aggregation and cellular toxicity. Proc Natl Acad Sci USA, 105, 6439-44.
21. Buratti, E. et al. (2001) Characterization and functional implications of the RNA binding properties of nuclear factor TDP-43, a novel splicing regulator of CFTR exon 9. J Biol Chem, 276, 36337-43.
22. Ayala, Y. M., et al. (2005) Human, Drosophila, and C. elegans TDP43: nucleic acid binding properties and splicing regulatory function. J Mol Biol, 348, 575-88.
23. Lessing, D. et al. (2009) Maintaining the brain: insight into human neurodegeneration from Drosophila melanogaster mutants. Nat Rev Genet.
24. Lu, B. et al. (2009) Drosophila models of neurodegenerative diseases. Annu Rev Pathol, 4, 315-42.
25. Shulman, J. M., et al. (2003) From fruit fly to bedside: translating lessons from Drosophila models of neurodegenerative disease. Curr Opin Neurol, 16, 443-9.
26. Jin, P., et al. (2003) RNA-mediated neurodegeneration caused by the fragile X premutation rCGG repeats in Drosophila. Neuron, 39, 739-47.
27. Watson, M. R., et al. (2008) A drosophila model for amyotrophic lateral sclerosis reveals motor neuron damage by human SOD1. J Biol Chem, 283, 24972-81.
28. Lu, Y., et al. (2009) Frontotemporal dementia and amyotrophic lateral sclerosis-associated disease protein TDP-43 promotes dendritic branching. Mol Brain, 2, 30.
29. Budnik, V. (1996) Synapse maturation and structural plasticity at Drosophila neuromuscular junctions. Curr Opin Neurobiol, 6, 858-67.
30. Bronk, P., et al. (2005) The multiple functions of cysteine-string protein analyzed at Drosophila nerve terminals. J Neurosci, 25, 2204-14.
31. Bellen, H. J., et al. (2004) The BDGP gene disruption project: single transposon insertions associated with 40% of Drosophila genes. Genetics, 167, 761-81.
32. Le Bourg, E., et al. (1992) Hypergravity and aging in Drosophila melanogaster. 4. Climbing activity. Gerontology, 38, 59-64.
33. Feany, M. B., et al. (2000) A Drosophila model of Parkinson's disease. Nature, 404, 394-8.
34. Orso, G., et al. (2005) Disease-related phenotypes in a Drosophila model of hereditary spastic paraplegia are ameliorated by treatment with vinblastine. J Clin Invest, 115, 3026-34.
35. Gustafson, K., et al. (1996) Distinct expression patterns detected within individual tissues by the GAL4 enhancer trap technique. Genome, 39, 174-82.
36. Mackenzie, I. R., et al. (2008) The role of transactive response DNA-binding protein-43 in amyotrophic lateral sclerosis and frontotemporal dementia. Curr Opin Neurol, 21, 693-700.
37. Brand, A. H., et al. (1993) Targeted gene expression as a means of altering cell fates and generating dominant phenotypes. Development, 118, 401-15.
38. Bilen, J., et al. (2005) Drosophila as a model for human neurodegenerative disease. Annu Rev Genet, 39, 153-71.
39. Osterwalder, T., et al. (2001) A conditional tissue-specific transgene expression system using inducible GAL4. Proc Natl Acad Sci USA, 98, 12596-601.
40. Tanouye, M. A., et al. (1980) Motor outputs of giant nerve fiber in Drosophila. J Neurophysiol, 44, 405-21.
41. Gorczyca, M., et al. (1984) Identification of a cholinergic synapse in the giant fiber pathway of Drosophila using conditional mutations of acetylcholine synthesis. J Neurogenet, 1, 289-313.
42. Godenschwege, T. A., et al. (2002) Ectopic expression in the giant fiber system of Drosophila reveals distinct roles for roundabout (Robo), Robo2, and Robo3 in dendritic guidance and synaptic connectivity. J Neurosci, 22, 3117-29.
43. Zarnescu, D.C., et al. (2005) Fragile X protein functions with lgl and the par complex in flies and mice. Dev Cell, 8, 43-52.
44. Chang, S., et al. (2008) Identification of small molecules rescuing fragile X syndrome phenotypes in Drosophila. Nat Chem Biol, 4, 256-63.

### 3. Example 2

As described herein, the fruit fly Drosophila is a premiere genetic model for studying human disease. A Drosophila model of ALS has been developed based on the RNA binding protein TDP-43. Overexpression of human TDP-43 in neurons or glia leads to phenotypes that mimic ALS pathology including locomotor dysfunction and reduced survival. Using this model a drug screen was completed using 1200 FDA approved compounds and several compounds that rescue TDP-43 induced lethality in Drosophila were identified. Diverse drug classes ranging from anti-diabetics to anti-inflammatories to antibacterials were among those with neuroprotective potential in vivo. This study focuses on anti-diabetics and the mechanisms by which they rescue TDP-43 neurotoxicity. Although some drugs used for diabetes treatment (e.g., glitazones) and the insulin pathway have been previously explored for their therapeutic potential in neurodegeneration and aging, results have been mixed. To determine the role of insulin signaling in ALS and to assess its potential as a therapeutic target in neuromuscular disease the powerful genetic toolbox and ALS-like phenotypes of the Drosophila model can be used. This approach can be applied to wild-type or several mutant TDP variants, which can help develop allele specific therapeutics.

Determination of the neuroprotective effects of candidate anti-diabetic drugs in vivo. First, candidate anti-diabetic compounds can be administered at a range of concentrations to determine the optimal amount for rescuing TDP-43 induced lethality. Second, a battery of phenotypic assays can be used to determine what aspects of TDP-43 neurotoxicity are rescued by candidate anti-diabetic compounds. Third, combinations of different anti-diabetic drug categories can be used to test their ability to synergistically mitigate ALS phenotypes. These experiments can establish the efficacy of different known categories of anti-diabetic drugs, can determine a map of their phenotypic consequences in vivo and can assess the feasibility of developing therapeutic drug cocktails.

### a. Identification of Components of the Insulin/PI3K/Akt/TOR Signaling Pathway that Modulate the Neurotoxicity of TDP-43 In Vivo

The insulin pathway is dysregulated in a fly model based on TDP-43 and results indicate that TDP-43 exhibits genetic interactions with PI3K, Akt and S6K, three core components of the insulin pathway. Genetic interactions can be used to determine whether loss or gain of function for components of the insulin/PI3K/Akt/TOR signaling cassette can modulate known TDP-43 phenotypes in vivo. These experiments can establish critical nodes in the insulin signaling pathway that can serve as targets for modulating the neurotoxicity due to TDP-43.

A major confounding issue in drug discovery is the presence of off-target effects. The Drosophila TDP-43 model can be used to administer candidate drugs in the context of genetic alterations (loss or gain function) for the predicted molecular target of individual compounds or various components of the insulin pathway. These experiments can determine whether the candidate anti-diabetic compounds work through predicted components of insulin signaling or alternative targets. In addition, these results can identify specific segments within the insulin pathway that modulate the neuroprotective capacity of candidate compounds.

Using a 1200 panel of FDA approved drugs candidate anti-diabetic compounds have been identified that rescue the lethality due to TDP-43 overexpression in motor neurons, in vivo. These findings indicate that insulin signaling and energy homeostasis are dysregulated in the Drosophila model, which is consistent with previous literature reports that insulin resistance is associated with ALS and related neurodegenerative disorders. Indeed, genetic interaction experiments support the notion that the PI3K/Akt/TOR pathway is dysregulated upon TDP-43 overexpression in neurons. The rationale for testing the therapeutic potential of candidate anti-diabetic drugs in the Drosophila model is based on the following: (1) the fly model recapitulates several aspects of ALS pathology; (2) the insulin signaling pathway is conserved between flies and mammals; (3) drug screening is performed in vivo, in a relatively high-throughput manner; (4) the availability of a sophisticated genetic toolbox affords the ability to modulate gene dosage in a tissue specific manner (e.g., motor neurons); (5) the ability to perform in vivo target validation by administering drugs in the context of gene mutations for candidate drug targets. Taken together, these features combined with the short generation time and relatively inexpensive costs of fly cultures, provide a strong rationale for using the Drosophila model that has already generated therapeutic leads.

Significant efforts have been made to study ALS, primarily by using SOD1 models, which are representative of less than 1% of ALS cases. This can partially explain why candidate compounds selected mainly using SOD1 models have failed to identify therapeutically active compounds in human populations. Since TDP-43 is involved in the pathology of a majority of ALS cases and has been shown to also be a cause for the disease, studies of a TDP-43 based model can provide insights and strategies for a wide spectrum of ALS cases.

### b. Insulin Signaling was Linked to Reduced Energy Homeostasis and Aging, Two Major Risk Factors for ALS

Although neurodegenerative disorders such as Alzheimer's, Parkinson's, and Amyotrophic Lateral Sclerosis (ALS) have been linked to specific gene mutations, the onset of these diseases is clearly age dependent. It is thought that reduced tissue homeostasis and an increase in oxidative processes with age represent major risk factors for the decline in neuronal function and survival, which in turn affect locomotor function, sleep and cognition. All of these aging related processes have been shown to be intimately linked to insulin, which besides its effects on lowering glucose levels, has been shown to regulate lifespan, learning and memory (Cardoso et al., 2009). It is thought that energy homeostasis can be the key output of insulin signaling and dysregulation of this pathway is associated with a higher risk of neurodegeneration (Cardoso et al., 2009). Recently, glucose enrichment has been shown to rescue several worm models of neurodegeneration, however this neuroprotective effect came at the expense of a reduction in lifespan (Tauffenberger et al., 2012). These and other findings in the field support the notion that the insulin pathway acts as a “double edge sword” and underscore the importance of tightly regulating the output of insulin signaling (Cohen and Dillin, 2008).

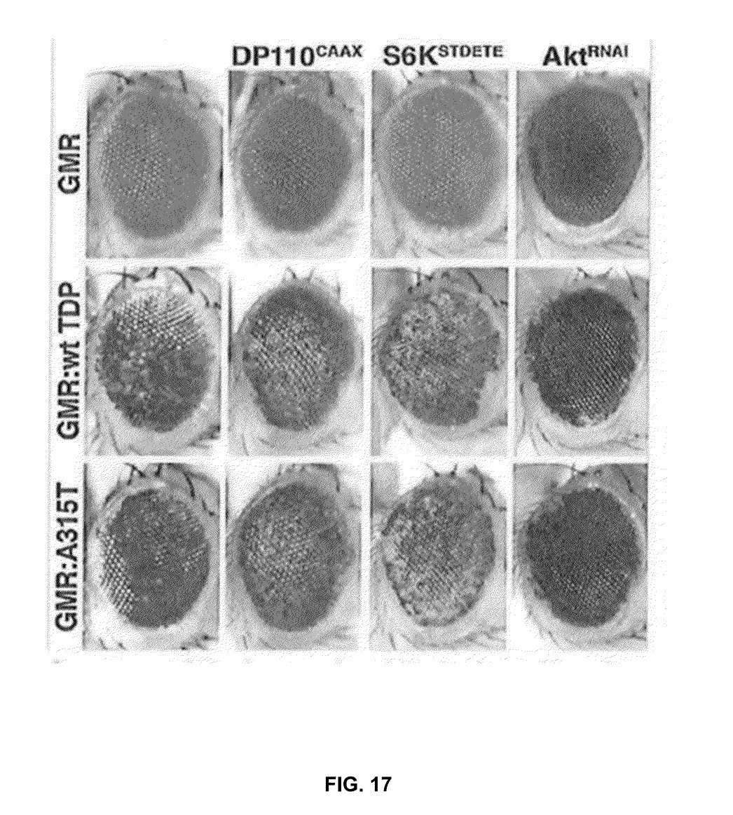

Using the Drosophila model of ALS, it was found that TDP-43 neurotoxicity has an aging component: neuronal loss and locomotor dysfunction are increased in older flies (Estes et al., 2011). Furthermore, functional interactions between TDP-43 and components of the insulin/PI3K/Akt/TOR pathway have been identified (FIG. 17). Together with the findings that select anti-diabetic drugs rescue TDP-43's neurotoxicity in vivo (Table 1), these results indicate that therapeutic strategies targeting the insulin signaling pathway can be effectively explored using a pharmacogenetic approach in this fly model.

### c. In Vivo Drug Screening Identified Several Classes of Compounds that Rescue TDP-43 Mediated Neurotoxicity

A drug screen aimed at rescuing the adult lethality caused by wild-type or mutant TDP-43 (D169G, G298S, A315T and N345K) overexpression in motor neurons has been performed (FIG. 11). 1200 FDA approved drugs from the Prestwick collection were fed to flies expressing TDP-43 at an initial concentration of 50 μM (as previously described in Chang et al., 2008). This screening strategy has several advantages including: (1) it is performed in vivo, (2) it is based on a robust phenotype (100% adult lethality), (3) it can lead to rapid identification of drugs that are already approved for use in humans and (4) it can inform the future development of small molecules with enhanced neuroprotective capabilities.

FIG. 11 shows that drug screen for compounds that rescue TDP-43 mediated neurotoxicity in vivo. FDA approved drugs from the Prestwick collection (200 mM in DMSO) are mixed with yeast-cornmeal based food and a bromophenol blue (as a mixing indicator) at a final concentration of 50 μM. Larvae expressing TDP-43 (wild type or mutant, as shown) are raised on drug containing food. In the absence of drugs, TDP-43 expression is lethal at the pupal/pharate stage. Drugs that rescued TDP-43 Induced Lethality (anywhere from 1-12 adults) were considered as candidates for future studies.

### d

TABLE 1
CANDIDATE COMPOUNDS IDENTIFIED IN PRIMARY
SCREENING IN THE DROSOPHILA MODEL

| | | Rescue Effect | | |
|---|---|---|---|---|
| | | (out of 15-20 | | Droso- |
| Drug category | Rescued | pupae) | Molecular | phila |
| (Drug name) | Transgene | (drug conc.) | target(s) | homolog |
| Sulfonyurea | Wild-type | 1-2 adults | 1. ATP-sensitive | 1. Irk3 |
| (Acetohexamide, | TDP-43, | (50 μM) | inward rectifier | |
| Gliquidone) | | | potassium | |
| | | | channel 1 | |
| | G298S | 6-9 adults | 2. Sulfonylurea | 2. Sur |
| | | (25 μM) | receptor | |
| Biguanide | Wild-type | 2 adults | AMPK | SNF1A |
| (Metformin, | TDP-43 | (25 μM) | | |
| Phenformin) | | | | |
| Thiazolidinedione | Wild-type | 1-2 adults | PPAR gamma | Nuclear |
| (Pioglitazone, | TDP-43 | 50 (μM) | (lipid/glucose | receptors |
| Troglitazone) | | | metabolism) | E75, E78 |
| | | | mitoNEET | |
| | | | (oxidative stress) | |

Using the drug screening strategy shown in FIG. 11, six drugs approved by the FDA for the treatment of diabetes mellitus have been identified (Table 1). These six drugs fall into three distinct categories: sulfonylureas (Acetohexamide and Gliquidone), biguanides (Metformin and Phenformin), and thiazolidinediones/PPAR inhibitors (Pioglitazone and Troglitazone).

Although the number of rescues obtained were ranging from 1-9 adult flies (out of 15-20 pupae), the results indicate that 50 μM, the concentration used in the primary screen may have been too high as it only rescued 1-2 adults. Indeed, secondary screening at lower concentrations indicate that 10-30 μM is better tolerated and generates more surviving adults (Table 1). The candidate anti-diabetic compounds can be further validated and optimized by using the screening strategy shown in FIG. 11 in conjunction with a battery of phenotypic assays. Table 2 shows the preliminary rescue results for various compounds and transgenes.

### e

TABLE 2
PRELIMINARY RESCUE RESULTS BY COMPOUND

| | | | |
|---|---|---|---|
| Meptazinol Hydrochloride | G298S | 1 Adult Normal Wing | 30 μM |
| Rimexolone | WT2 | 1 Adult Normal Wing | 30 μM |
| Risperidone | WT2 | 1 Adult Normal Wing | 30 μM |
| Xamoterol HemifiμMarate | G298S | 1 Adult Normal Wing | 30 μM |
| Etilifrine Hydrochloride | WT2 | 1 Adult Normal Wing | 30 μM |
| Acamprosate Calcium | D169G | 1 Adult Normal Wings | 50 μM |
| Flufenamic Acid | G298S | 1 Adult Normal Wings | 50 μM |
| Phenazopyridine | WT2 | 1 Adult Normal Wings | 30 μM |
| Hydrochloride | | | |
| Pyrantel Tartrate | G298S | 1 Adult Normal Wings | 50 μM |
| Morantel Tartrate | WT2 | 1 Adult Normal Wings | 50 μM |
| Hycanthone | D169G | 1 Adult Normal Wings | 50 μM |
| Fluticasone Propionate | WT2 | 1 Adult Normal Wings | 30 μM |
| Celecoxib | WT2 | 1 Adult Normal Wings | 50 μM |
| Ibutilide FμMarate | G298S | 1 Adult Normal Wings | 50 μM |
| Clotrimazole | WT2 | 1 Adult Normal Wings | 50 μM |
| Minocycline | N345K | 1 Adult Normal Wings | 50 μM |
| Sulfisoxazole | WT2 | 1 Adult Normal Wings | 50 μM |
| Dicloxacillin Sodium Salt | WT2 | 1 Adult Normal Wings | 50 μM |
| Sisomicin Sulfate | WT2 | 1 Adult Normal Wings | 50 μM |
| Cinoxacin | G298S | 1 Adult Normal Wings | 30 μM |
| Azlocillin Na Salt | WT2 | 1 Adult Normal Wings | 30 μM |
| Cefoxitin Sodium Salt | WT2 | 1 Adult Normal Wings | 30 μM |
| Roxitromycin | WT2 | 1 Adult Normal Wings | 30 μM |
| Sulbactam | WT2 | 1 Adult Normal Wings | 30 μM |
| Rufloxacin | WT2 | 1 Adult Normal Wings | 30 μM |
| Sulfanilamide | WT2 | 1 Adult Normal Wings | 50 μM |
| Prothionamide | WT2 | 1 Adult Normal Wings | 30 μM |
| Ticarcillin Sodiim | WT2 | 1 Adult Normal Wings | 30 μM |
| Azithromycin | WT2 | 1 Adult Normal Wings | 30 μM |
| Chenodiol | G298S | 1 Adult Normal Wings | 50 μM |
| Primidone | WT2 | 1 Adult Normal Wings | 30 μM |
| Amitryptiline Hydrochloride | G298S | 1 Adult Normal Wings | 50 μM |
| Trazodone | G298S | 1 Adult Normal Wings | 50 μM |
| Venlafaxine | G298S | 1 Adult Normal Wings | 50 μM |
| Protriptyline Hydrochloride | WT2 | 1 Adult Normal Wings | 30 μM |
| Minaprine Dihydrochloride | WT2 | 1 Adult Normal Wings | 50 μM |
| Pioglitazone | WT2 | 1 Adult Normal Wings | 50 μM |
| Repaglinide | WT2 | 1 Adult Normal Wings | 50 μM |
| Acetohexamide | WT2 | 1 Adult Normal Wings | 50 μM |
| Cyclizine Hcl | WT2 | 1 Adult Normal Wings | 50 μM |
| Ondansetron Hcl | WT2 | 1 Adult Normal Wings | 30 μM |
| Fluconazole | WT2 | 1 Adult Normal Wings | 50 μM |
| Propoxycaine Hcl | WT2 | 1 Adult Normal Wings | 30 μM |
| Bifonazole | G298S4 | 1 Adult Normal Wings | 30 μM |
| Pheniramine Maleate | G298S | 1 Adult Normal Wings | 50 μM |
| Orphenadrine | G298S | 1 Adult Normal Wings | 50 μM |
| Carbinoxamine Maleate Salt | WT2 | 1 Adult Normal Wings | 30 μM |
| Chlorpheniramine Maleate | WT2 | 1 Adult Normal Wings | 50 μM |
| Fexofenadine Hcl | WT2 | 1 Adult Normal Wings | 30 μM |
| Atorvastatin | WT2 | 1 Adult Normal Wings | 30 μM |
| Urapidil Hydrochloride | WT2 | 1 Adult Normal Wings | 30 μM |
| Prazosin Hydrochloride | WT2 | 1 Adult Normal Wings | 30 μM |
| Cyclopenthiazide | WT2 | 1 Adult Normal Wings | 30 μM |
| Penbutolol Sulfate | WT2 | 1 Adult Normal Wings | 30 μM |
| Oxprenolol Hcl | WT2 | 1 Adult Normal Wings | 30 μM |
| Ramipril | WT2 | 1 Adult Normal Wings | 30 μM |
| Carvedilol | WT2 | 1 Adult Normal Wings | 30 μM |
| Amrisentan | WT2 | 1 Adult Normal Wings | 50 μM |
| Pargyline Hydrochloride | WT2 | 1 Adult Normal Wings | 50 μM |
| Guanethidine | WT2 | 1 Adult Normal Wings | 50 μM |
| Beoprolol Fumarate | WT2 | 1 Adult Normal Wings | 50 μM |
| Doxazosin Mesylate | WT2 | 1 Adult Normal Wings | 30 μM |
| Methimazole | WT2 | 1 Adult Normal Wings | 30 μM |
| Ketoprofen | WT2 | 1 Adult Normal Wings | 50 μM |
| Felbinac | WT2 | 1 Adult Normal Wings | 30 μM |
| Deoxycorticosterone | WT2 | 1 Adult Normal Wings | 30 μM |
| Oxphenbutazone | WT2 | 1 Adult Normal Wings | 30 μM |
| Clofibrate | D169G | 1 Adult Normal Wings | 50 μM |
| Primaquine Diphosphate | WT2 | 1 Adult Normal Wings | 50 μM |
| Carbadox | WT2 | 1 Adult Normal Wings | 50 μM |
| Ronidazole | G298S | 1 Adult Normal Wings | 30 μM |
| Toremifene | WT2 | 1 Adult Normal Wings | 30 μM |
| Vatalanib | D169G | 1 Adult Normal Wings | 50 μM |
| Procyclidine HCL | WT2 | 1 Adult Normal Wings | 30 μM |
| Anthralin | WT2 | 1 Adult Normal Wings | 50 μM |
| Triflupromazine | G298S | 1 Adult Normal Wings | 50 μM |
| Sulpiride | WT2 | 1 Adult Normal Wings | 50 μM |
| Thioproperazine Dimesylate | WT2 | 1 Adult Normal Wings | 50 μM |
| Clozapine | G298S | 1 Adult Normal Wings | 50 μM |
| Piperacetazine | WT2 | 1 Adult Normal Wings | 30 μM |
| Chloroxine | D169G | 1 Adult Normal Wings | 50 μM |
| Hexachlorophene | WT2 | 1 Adult Normal Wings | 30 μM |
| Cloperastine HCl | WT2 | 1 Adult Normal Wings | 30 μM |
| Abacavir Sulfate | G298S | 1 Adult Normal Wings | 30 μM |
| Tracazolate Hydrochloride | WT2 | 1 Adult Normal Wings | 30 μM |
| Zaleplon | WT2 | 1 Adult Normal Wings | 50 μM |
| Zardaverine | WT2 | 1 Adult Normal Wings | 30 μM |
| Amrinone | WT2 | 1 Adult Normal Wings | 30 μM |
| Amifostine | G298S | 1 Adult Normal Wings | 50 μM |
| Gefitinib | G298S | 1 Adult Normal Wings | 50 μM |
| Iopromide | WT2 | 1 Adult Normal Wings | 30 μM |
| Luteolin | WT2 | 1 Adult Normal Wings | 30 μM |
| Amiprilose HCl | WT2 | 1 Adult Normal Wings | 30 μM |
| Methantheline Bromide | WT2 | 1 Adult Normal Wings | 30 μM |
| Eucatropine HCl | WT2 | 1 Adult Normal Wings | 30 μM |
| Meprylcaine HCl | WT2 | 1 Adult Normal Wings | 30 μM |
| Benoxinate Hydrochloride | WT2 | 1 Adult Normal Wings | 50 μM |
| Dibucaine | WT2 | 1 Adult Normal Wings | 50 μM |
| Dehydroisoandosterone 3- | WT2 | 1 Adult Normal Wings | 30 μM |
| Acetate | | | |
| Tyloxapol | D169G | 1 Adult Normal Wings | 30 μM |
| Mephenesin | G298S | 1 Adult Normal Wings | 50 μM |
| N-Acetyl-L-Leucine | WT2 | 1 Adult Normal Wings | 30 μM |
| Rolipram | WT2 | 1 Adult Normal Wings | 30 μM |
| Fipexide Hydrochloride | WT2 | 1 Adult Normal Wings | 50 μM |
| Hemicholinium Bromide | G298S | 1 Adult Normal Wings | 50 μM |
| Ethamivan | WT2 | 1 Adult Normal Wings | 50 μM |
| Butalbital | WT2 | 1 Adult Normal Wings | 50 μM |
| Chlormezanone | N345K | 1 Adult Normal Wings | 50 μM |
| Cyclobenzaprine HCl | D169G | 1 Adult Normal Wings | 50 μM |
| Drofenine HCl | WT2 | 1 Adult Normal Wings | 30 μM |
| Verteporfin | WT2 | 1 Adult Normal Wings | 30 μM |
| Oxybenzone | WT2 | 1 Adult Normal Wings | 30 μM |
| Estradiol Valerate | WT2 | 1 Adult Normal Wings | 30 μM |
| Salmeterol | WT2/D169G | 1 Adult Normal Wings × 2 | 30 μM |
| Estradiol 17 Beta | D169G, A315T | 1/1 Adult Normal Wings | 50 μM |
| Proglumide | WT2/G298S | 1 Adult Normal Wings, | 50 μM |
| | | 3 Adults Normal Wings | |
| Phenylpropanolamine | WT2 | 1 Adult: 1 Normal Wing | 50 μM |
| Hydrochloride | | 1 Folded Wing | |
| Acetylsalicylic Acid | WT2 | 1/1 Adult Normal Wings | 30 μM |
| Amcinonide | WT2 | 1/1 Adult Normal Wings | 30 μM |
| Acetylcysteine | D169G/G298S | 1/1 Adult Normal Wings | 30 μM |
| Ibudilast | WT2 | 1/1 Adult Normal Wings | 30 μM |
| Cefdinir | WT2 | 1/2 Adult Normal Wings | 50 μM |
| Romhexine Hydrochloride | G298S | 1/2 Adult Normal Wings | 30 μM |
| Ketorolac Tromethamine | WT2 | 2 Adult Normal Wings | 30 μM |
| BepheniμM | WT2 | 2 Adult Normal Wings | 30 μM |
| Hydroxynaphthoate | | | |
| Mepenzolate Bromide | G298S | 2 Adult Normal Wings | 50 μM |
| Sulfamethazine Na Salt | WT2 | 2 Adult Normal Wings | 30 μM |
| Cefepime Hydrochloride | WT2 | 2 Adult Normal Wings | 30 μM |
| Urosiol | WT2 | 2 Adult Normal Wings | 30 μM |
| Dichlorphenamide | WT2 | 2 Adult Normal Wings | 30 μM |
| Clofibric Acid | WT2 | 2 Adult Normal Wings | 30 μM |
| Quinapril Hcl | WT2 | 2 Adult Normal Wings | 30 μM |
| Cilnidipine | WT2 | 2 Adult Normal Wings | 30 μM |
| Fenoprofen Ca Salt | WT2 | 2 Adult Normal Wings | 30 μM |
| Dihydrate | | | |
| Alclometasone Dipropionate | WT2 | 2 Adult Normal Wings | 30 μM |
| R-Naproxen Na Salt | WT2 | 2 Adult Normal Wings | 30 μM |
| Suprofen | WT2 | 2 Adult Normal Wings | 30 μM |
| Prednicarbate | WT2 | 2 Adult Normal Wings | 30 μM |
| Levopropoxyphene | WT2 | 2 Adult Normal Wings | 30 μM |
| Napsylate | | | |
| Nizatidine | WT2 | 2 Adult Normal Wings | 30 μM |
| Imiquimod | WT2 | 2 Adult Normal Wings | 30 μM |
| Tetracaine Hydrochloride | A315T | 2 Adult Normal Wings | 50 μM |
| Methylatropine Nitrate | WT2 | 2 Adult Normal Wings | 30 μM |
| Benztropine Mesylate | WT2 | 2 Adult Normal Wings | 30 μM |
| Closantel | WT2 | 2 Adults Normal Wings | 50 μM |
| Troglitazone | WT2 | 2 Adults Normal Wings | 50 μM |
| Tioconazole | A315T | 2 Adults Normal Wings | 50 μM |
| Carteolol HCl | WT2 | 2 Adults Normal Wings | 50 μM |
| Fludrocortisone | WT2 | 2 Adults Normal Wings | 50 μM |
| Clobutinol HCl | WT2 | 2 Adults Normal Wings | 50 μM |
| Lamivudine | G298S | 2 Adults Normal Wings | 50 μM |
| Metrizamide | WT2 | 2 Adults Normal Wings | 50 μM |
| Raclopride | WT2 | 2 Adults Normal Wings | 50 μM |
| Ritodrine | WT2 | 2 Adults Normal Wings | 50 μM |
| Methenamine | WT2/G298S | 2/1 Adult Normal Wings | 30 μM |
| Valproic Acid | WT2/G298S | 2/1 Adult Normal Wings | 30 μM |
| Mephenytoin | WT2 | 2/1 Adult Normal Wings | 30 μM |
| R-(+)-Atenolol | WT2/D169G | 2/1 Adult Normal Wings | 30 μM |
| Parbendazole | WT2 | 2/1 Adult Normal Wings | 30 μM |
| Diclazuril | WT2/G298S | 2/1 Adult Normal Wings | 30 μM |
| ′Telmisartan | WT2 | 2/2 Adult Normal Wings | 30 μM |
| Cefamandole Na Salt | WT2 | 2/3 Adult Normal Wing | 30 μM |
| Zomepirac Na Salt | WT2 | 3 Adult Normal Wings | 30 μM |
| Stanozolol | D169G | 3 Adult Normal Wings | 50 μM |
| Niridazole | WT2 | 3 Adult Normal Wings | 50 μM |
| Sulfameter | WT2 | 3 Adult Normal Wings | 30 μM |
| Tosufloxacin Hydrochloride | WT2 | 3 Adult Normal Wings | 50 μM |
| Enoxacin | WT2 | 3 Adult Normal Wings | 50 μM |
| DicμMarol | WT2 | 3 Adult Normal Wings | 30 μM |
| Levetiracetam | A315T | 3 Adult Normal Wings | 50 μM |
| Terazosin HC1 | WT2 | 3 Adult Normal Wings | 30 μM |
| Ribavirin | WT2 | 3 Adult Normal Wings | 30 μM |
| Clodronate | WT2 | 3 Adult Normal Wings | 50 μM |
| Iopamidol | WT2 | 3 Adult Normal Wings | 30 μM |
| Vardenafil | WT2 | 3 Adult Normal Wings | 50 μM |
| Pentobarbital | A315T | 3 Adult Normal Wings | 50 μM |
| Liothyronine | WT2 | 3 Adult Normal Wings | 30 μM |
| Darifenacin Hydrobromide | WT2 | 3 Adult Normal Wings | 30 μM |
| Idebenone | WT2 | 3 Adults Normal Wings | 50 μM |
| Chlorprothixene | WT2 | 3 Adults Normal Wings | 50 μM |
| Homochlorcyclizine | WT2/A315T | 3 Adults Normal Wings, | 50 μM |
| | | 1 Adult Normal Wings | |
| Fleroxacin | WT2/G298S | 3/1 Adult Normal Wings | 30 μM |
| Lacidipine | WT2/G298S | 3/7 Adult Normal Wings | 50 μM |
| Clinafloxacin | WT2 | 4 Adult Normal Wings | 30 μM |
| Mesna | WT2 | 4 Adult Normal Wings | 50 μM |
| Tacrine Hydrochloride | WT2 | 4 Adults Normal Wings | 50 μM |
| Hydrate | | | |
| Melatonin | WT2 | 4 Adults Normal Wings | 50 μM |
| Gabazine | WT2/G298S | 4 Adults Normal Wings, | 50 μM |
| | | 2 Adults Straight Wings | |
| Entacapone | G298S | 5 Adult Normal Wings | 50 μM |
| Ethynyletradiol 3-Methyl | WT2 | 5 Adult Normal Wings | 30 μM |
| Ether | | | |
| (+,−)-Octopamine HCl | WT2 | 5 Adult Normal Wings | 30 μM |
| Hydrocortisone Base | WT2 | 5 Adults Straight Wings | 50 μM |
| Neostigmine Bromide | WT2 | 5 Adults Straight Wings | 50 μM |
| Alexidine Dihydrochloride | WT2 | 6 Adults Normal Wings | 30 μM |
| Meclozine Dihcl | D169G | 6 Adults Normal Wings | 50 μM |
| Hexylcaine HCl | WT2 | 6 Adults Normal Wings | 30 μM |
| Suloctidil | WT2 | 6 Adults Normal Wings | 50 μM |
| Gliquidone | WT2 | 7 Adult Normal Wings | 30 μM |
| Lithocholic Acid | WT2 | 7 Adult Normal Wings | 30 μM |
| Podophyllotoxin | WT2 | 8 Adult Normal Wings | 30 μM |
| Nilutamide | WT2 | 11 Adult Normal Wings | 30 μM |
| Perhexiline Maleate | N345K | 12 Adults | 50 μM |
| Dioxybenzone | WT2 | 14 Adult Normal Wings | 30 μM |

In Table 2, the following apply: WT=wild-type TDP-43 and D169G, G28S, A315T, and N345K are disease variants.

Table 3 shows a summary of the preliminary rescue results grouped by number of adults rescued.

TABLE 3
SUMMARY OF PRELIMINARY RESCUE RESULTS

| # Compounds | # Adults Rescued | % of Drugs |
|---|---|---|
| 203 | >1 | 16.92 |
| 123 | 1 | 10.25 |
| 80 | >2 | 6.67 |
| 41 | >3 | 3.42 |
| 21 | >4 | 1.75 |

### f

### g. TDP-43 Associated with Stress Granules in Cultured Motor Neurons

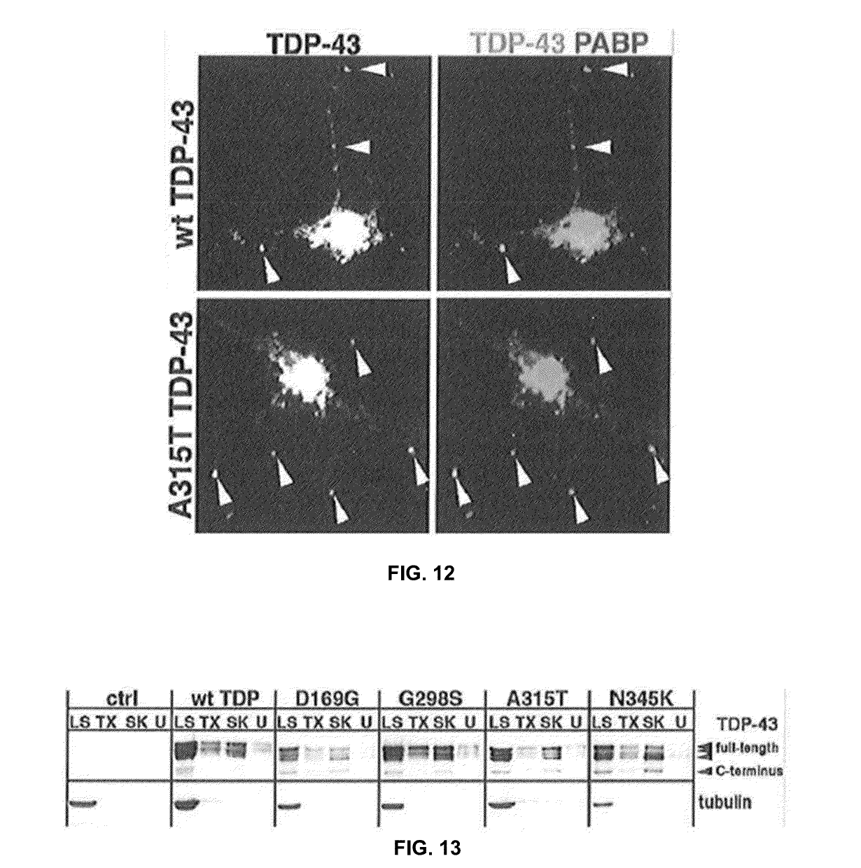

Recently, TDP-43 mutations have been shown to associate with stress granules in various cell lines subjected to cellular stress (Dewey et al., 2011; Liu-Yesucevitz et al., 2010; McDonald et al., 2011). RNA granules play critical roles in post-transcriptional gene expression and are comprised of large RNA/protein complexes that can be visualized as cytoplasmic puncta (FIG. 12). Stress granules are foci of RNA storage that form in response to cellular stress and correlate with translation inhibition. Using cultured motor neurons from Drosophila ventral ganglia, evidence that wt and A315T TDP-43 exhibited extensive co-localization with PolyA Binding Protein (PABP), a stress granule marker, has been obtained (FIG. 12). A315T appeared to co-localize nearly perfectly (arrowheads), while wild-type TDP-43 only partially overlapped with PABP. Recent evidence indicates that TDP-43 containing stress granules can mature into ubiquitin positive aggregates, a hallmark of ALS pathology (Parker et al., 2012). These results indicate that TDP-43's function extends beyond splicing and position TDP-43 as a player in multiple aspects of RNA regulation, including stress response in neurons.

FIG. 12 shows that TDP-43 co-localized with PABP in neuronal stress granules. Genotypes indicated on the left. Stainings indicated on the top. Arrowheads indicate granules containing both TDP-43 and PABP (right panels). TDP-43 was visualized via YFP tag. Cell bodies were saturated to allow visualization of the smaller RNA granules within neurites.

### h. TDP-43 Co-Fractionated with Detergent Insoluble Aggregates

A characteristic biochemical feature of ALS and related neurodegenerative disorders is the formation of detergent-insoluble TDP-43 aggregates and truncated C-terminus fragments (Neumann et al., 2006). To test whether the Drosophila model recapitulates this hallmark of ALS pathology flies expressing TDP-43 were subjected to cellular fractionations using buffers of increased solubilizing strength (Liachko et al., 2010) (FIG. 13).

FIG. 13 shows that TDP-43 co-fractionated with insoluble complexes. Cellular fractionation of adult head tissue shows that TDP-43 was found in all cellular fractions including the soluble (LS, Low Salt), Triton-X100 (TX, nonionic detergent) as well as sarkosyl (SK, ionic detergent) and detergent insoluble fraction (U, urea). All TDP variants exhibit high molecular weight forms corresponding to posttranslational modifications (top arrowheads, full-length) as well as truncated C terminal fragments (bottom arrowhead, C-terminus). Tubulin was used as a loading control. TDP-43 transgenes used are C-terminal tagged with YFP. Western blot was performed using anti-GFP or tubulin antibodies as appropriate. Indeed, all TDP variants including wild-type and four mutant forms linked to ALS in human patients exhibited some amount of insoluble TDP-43 (U, urea fraction) as well as a smaller C terminal fragment of approximately 25 kDa (bottom arrowhead in FIG. 13).

### i. TDP-43 Overexpression in Motor Neurons Altered Neuromuscular Junction (NMJ) Growth and Function

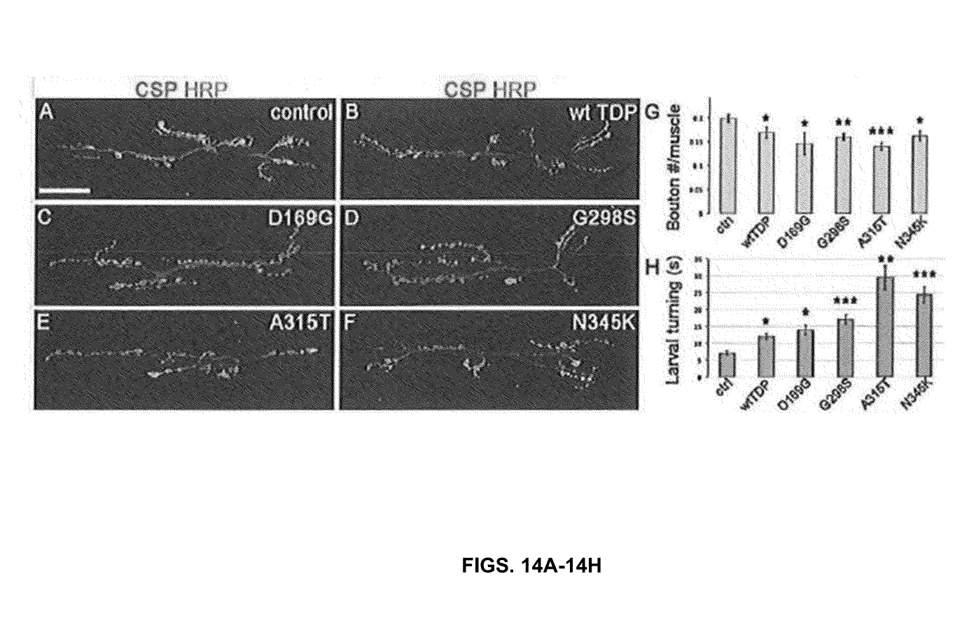

It has been shown that TDP-43 is required for the architecture of the larval NMJ (Estes et al., 2011; Feiguin et al., 2009; Li et al., 2010), a well-established model for studies of synaptic development and function (Koh et al., 2000). The larval NMJ synapse has been studied at muscles 6/7, which consists of structural varicosities referred to as synaptic boutons that form when motor neuron terminals innervate the surface of post-synaptic muscles (Koh et al., 2000). The pre-synaptic motor neuron membrane was labeled at the neuromuscular junction with Horseradish Peroxidase (HRP) antibodies and the synaptic vesicles within boutons with Cysteine String Protein antibodies (CSP, Ranjan et al., 1998) (FIG. 14A-F). Using these markers the effect of TDP-43 variants on the overall size of the synapse was measured by quantifying the total number of boutons (as marked by HRP). When expressed in motor neurons (with D42 Gal4) it was found that all TDP-43 variants (wild-type, D169G, G298S, A315T and N345K) resulted in smaller synapses as indicated by a reduced number of synaptic boutons (FIG. 14G). This anatomical phenotype is accompanied by an impairment in locomotor function as indicated by a significant increase in larval turning time compared to controls (FIG. 14H).

FIG. 14 shows that NMJ morphology and locomotor function were altered when TDP-43 is overexpressed in motor neurons. (FIG. 14A-F) D42 Gal4 driven TDP-43 results in decreased number of synaptic boutons (CSP, HRP, as indicated). (FIG. 14G) Quantification of synaptic boutons indicated a reduction in synapse size due to TDP-43 overexpression. (H) Larvae expressing TDP-43 in motor neurons exhibited a significant increase in larval turning time, a well-established behavioral assays used to determine locomotor function. Student's T test was used to calculate significance. *=Pᵥₐₗᵤₑ<0.5; **=Pᵥₐₗᵤₑ<0.01; ***=Pᵥₐₗᵤₑ<0.001. Scale bar in (FIG. 14A): 3 μm. Synaptic marker distribution was affected by TDP-43 expression in motor neurons

To further investigate the molecular mechanisms underlying TDP-43 neurotoxicity in motor neurons, the distribution of synaptic markers, including Bruchpilot, (a core component of active zones recognized by the NC82 antibody, Kittel et al., 2006; Wagh et al., 2006) and the glutamate receptor GluRIIC (Marrus and DiAntonio, 2004) was examined.

FIG. 15A-H shows that the ratio between presynaptic active zones (AZ) and post synaptic glutamate receptors was affected by motor neuron expression of TDP-43.

D42 driven TDP-43 preferentially increased the number of presynaptic active zones (AZ) per bouton (quantification in FIG. 15G) without a similar increase in postsynaptic GluR (except for wt TDP, FIG. 15H). Stainings as indicated. Student's T test was used to calculate significance. *=P value<0.5; **=P value<0.01; ***=P value<0.001. Scale bar in (FIG. 15A): 5 μm.

As shown in FIG. 15, TDP-43 overexpression leads to a significant increase in active zones, which correspond to areas of neurotransmitter release (NC82 stainings, FIG. 15A-G). In contrast, TDP-43 had no significant effect on GluR levels (as measured by quantitative confocal fluorescence), except for wild-type TDP (FIG. 15H). These data indicate that the function of NMJ synapses was impaired due to TDP-43 overexpression in motor neurons and provide an explanation for the larval turning defect shown in FIG. 14G.

### j. Sleep and Locomotor Activity were Impaired in Adult Flies Expressing TDP-43 in the Nervous System

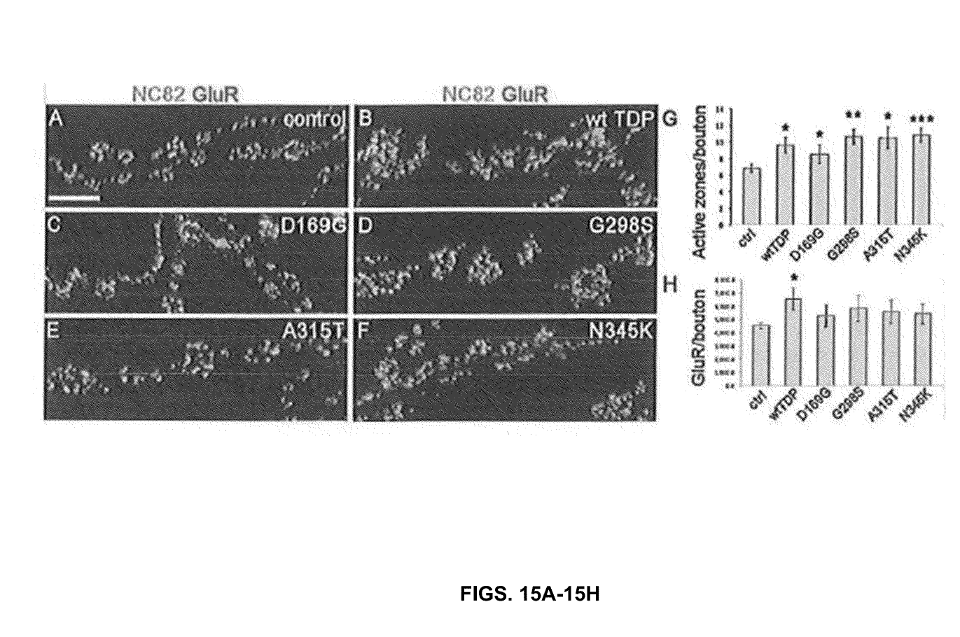

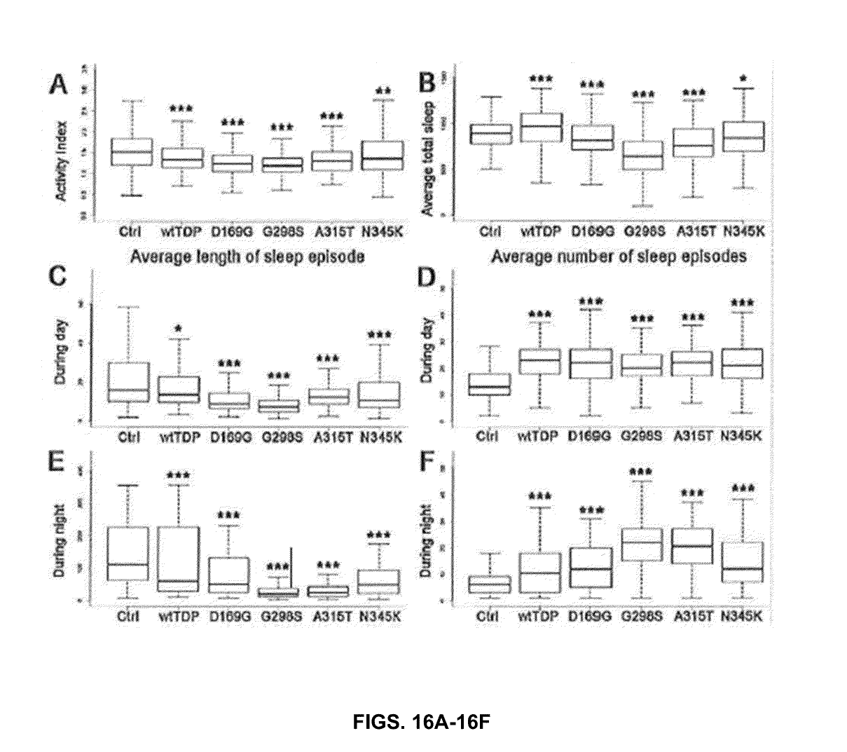

In addition to impaired motor function, ALS patients exhibit sleep disturbances. To determine whether TDP-43 affects sleep patterns in the fly model Drosophila Activity Monitors (DAMs) were used and quantified both locomotor and sleep activity in adult flies expressing TDP-43. DAMs can hold 32 long and narrow glass tubes filled with food on one end, plugged with cotton at the other end and housing one fly each. As a fly walks back and forth within its tube, it interrupts an infrared beam that crossed the tube at its midpoint and this interruption, detected by the onboard electronics, is added to the tube's activity count as a measure of fly activity. DAMs are kept at 25° C. in an incubator equipped with 12 hr alternating dark-light cycles. While each monitor can evaluate the behavior of 32 flies, several monitors can be simultaneously recorded from and thus hundreds of flies can be easily studied. This daily record provides a good measure of both the intensity of locomotor activity, and the relative periods of rest. A period of rest of 5 min or more is defined as sleep (Sehgal and Mignot, 2011). Activity can be evaluated as an average over a period of time for individuals or for populations of flies using Pysolo, a multi-platform software for analyzing sleep and locomotor activity in Drosophila (Gilestro and Cirelli, 2009). As shown in FIG. 16, TDP-43 expression alters both locomotor activity as well as sleep. While there are on average shorter sleep episodes both during day and night, adults expressing TDP-43 variants exhibit significantly more sleep episodes, consistent with the notion that their sleep is fragmented, possibly due to hyperexcitability.

FIG. 16 shows that locomotor activity was reduced and sleep patterns were altered in adults expressing TDP-43 (FIG. 16A-F). TDP-43 variants (as shown) were expressed with D42 Gal4. Measurements using DAMs show that locomotor activity was significantly reduced overall (A) and total sleep was significantly altered (B). WT TDP-43 expressing flies slept overall longer than the mutant variants. During the day (C, D) as well as during nighttime (E, F), TDP-43 expression resulted in significantly more sleep episodes that last shorter amounts of time. To determine significance, a non-parametric Wilcox test was calculated in R. In these experiments TDP-43 transgenics were used with lower levels of expression that do not affect adult viability. This is in contrast to the transgenics employed in the drug screen where high levels of expression lead to adult lethality (FIG. 11).

### k. the Insulin Signaling Pathway Modulates TDP-43's Neurotoxicity in Vivo

The ability of anti-diabetic drugs to rescue the lethality induced by TDP-43 overexpression in motor neurons indicates that the insulin signaling pathway is dysregulated in the fly model. Genetic interactions were performed between TDP-43 and select components of the insulin signaling pathway. Activated PI3K (DP110^{CAAX}) as well as constitutively active S6K^{STDETE}, a readout of TOR activation enhance the TDP-43 induced neurodegeneration in the retina (Barcelo and Stewart, 2002; Leevers et al., 1996) (FIG. 17). These data indicate that the PI3K/Akt/TOR pathway was hyperactivated due to TDP-43 overexpression. To further probe this scenario, an RNAi construct for Akt was overexpressed and it was found that attenuating the signaling pathway leads to a visible rescue of TDP-43 phenotypes in the eye. These experiments provided a rapid assessment of the potential for in vivo interactions between TDP-43 and the insulin pathway. These genetic interactions can be tested with additional components of the insulin/PI3K/Akt/TOR pathway in the retina as well as in motor neurons.

FIG. 17 shows that the insulin signaling pathway modulated TOP-43's neurotoxicity in vivo. Constitutively active PI3K and S6K overexpression enhanced while Akt reduction by RNAi mitigated TDP-43 induced neurodegeneration. Genotypes as indicated. GMR Gal4 was used to drive expression in the retina. Note that overexpression of DP110^{CAAX} and S6K5^{DETE} alone did not exhibit visible phenotypes (top row), indicating that the observed enhancements were synergistic and not additive.

### l. Summary of Results for Example 2

A Drosophila model of ALS based on TDP-43, an RNA binding protein linked to the majority of ALS cases known to date, has been developed. When expressed in motor neurons, TDP-43 generates phenotypes that recapitulate several hallmark features of the human disease (FIGS. 12-16). Using this model 1200, FDA approved drugs were screened and several anti-diabetic drugs were identified that rescue TDP-43 induced lethality (FIG. 11). These drugs fall into three distinct categories including sulfonylureas, biguanides and thiazolidinediones and target various aspects of insulin signaling to ultimately improve energy homeostasis (Table 1). These findings indicate that the insulin pathway is dysregulated due to TDP-43 overexpression in motor neurons and indicates that the insulin signaling pathway can be a therapeutic target. Indeed, genetic interaction experiments indicate that several components of the insulin/PI3K/Akt/TOR pathway exhibit functional interactions with TDP-43 in vivo (FIG. 17). To determine the role of the insulin pathway in ALS pathology and to establish the therapeutic potential of anti-diabetic drugs a combined pharmacogenetic approach in vivo, using the Drosophila model based on TDP-43 can be used.

### 4. Experimental Design for Example 2

### a. Determination of the Neuroprotective Effects of Candidate Antidiuretic Drugs In Vivo Rationale

An estimated 75% of human disease genes have homologues in Drosophila (Pandey and Nichols, 2011). Thus, disease modeling in the fly has led to the identification of important therapeutic leads for several human disorders including Fragile X syndrome (Chang et al., 2008), multiple endocrine neoplasia (Vidal et al., 2005) and Huntington's disease among others (reviewed in Rudrapatna et al., 2012). The integration of fly models into unbiased strategies for therapeutic discovery holds great promise due to the high degree of conservation between pathways, the availability of an unparalleled genetic toolbox and relative low costs compared to mammalian systems (reviewed in Pandey and Nichols, 2011). The fly model of ALS based on TDP-43 has these advantages, exhibits remarkable similarities with the human disease, and has proven fruitful in identifying promising candidate compounds such as anti-diabetic drugs that mitigate TDP-43's neurotoxicity in vivo.

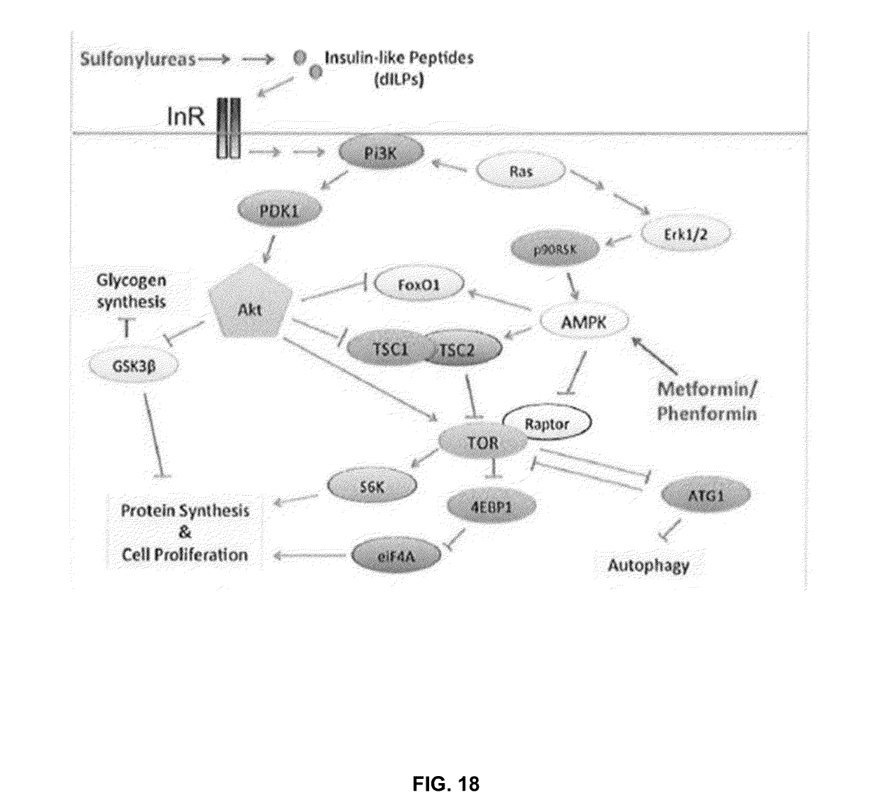

FIG. 18 shows that the insulin signaling pathway and the molecular targets of candidate anti-diabetic drugs are conserved in Drosophila. Sulfonylureas target ATP-dependent potassium channels K_{ATP} comprised of Inner Rectifier Potassium channel Irk3 in association with the sulfonylurea receptor (Sur). As a result of K_{ATP} activation on beta cells, the pancreas undergoes a surge in Ca²⁺ that mimics glucose entry and releases more insulin. In Drosophila, there are seven insulin like peptides (shown as diLPs), which initiate a signaling cascade upon binding to the insulin receptor (1 nR). For simplicity, only core components of the pathway are shown. Biguanides such as Metformin and Phenformin stimulate AMPK, an energy sensor, which in turn inhibits TOR activation. Shaded grey boxes indicate various cellular outcomes of the insulin/PI3K/Akt/TOR signaling.

First, optimization studies can be performed. To confirm the use of anti-diabetic drugs therapeutics for ALS, the optimum in vivo concentration of the compounds identified in primary screening is determined. Second, a battery of phenotypic assays can be used (FIGS. 12-16) to determine the efficacy of individual compounds in rescuing different aspects of TDP-43 neurotoxicity. Third, combinations of sulfonylureas and biguanides can be tested to determine whether they can act synergistically to rescue TDP-43 induced phenotypes. These experiments can determine the optimal concentration, can provide insights into the mechanism(s) by which the candidate anti-diabetic drugs act to rescue ALS phenotypes in the fly and can assess the feasibility of developing a therapeutic cocktail that targets insulin signaling and energy homeostasis.

The candidate compounds identified in primary screening (at 50 μM) can be tested for their ability to rescue TDP-43's adult lethality phenotype when delivered at concentrations ranging from 1 to 100 μM in fly food. These experiments can determine an optimal concentration that can generate the highest number of adults rescued upon TDP-43 expression in motor neurons using the D42 Gal4 driver (FIG. 11). A second round of optimization experiments can be conducted to determine additional drug concentrations around the best rescuing values and to establish a dose response curve. HPLC can be used to determine the precise concentration of the drug in the brain and the rest of the body.

Second, validation studies can be performed. A battery of phenotypes resulting from TDP-43 overexpression in motor neurons in vivo (Estes et al., 2011) and in vitro, in cultured neurons (FIGS. 12-16) has been established. These include TDP-43's association with neuronal stress granules (FIG. 12), with insoluble aggregates (FIG. 13), larval locomotor impairment (FIG. 14), defects in the architecture and function of neuromuscular junction synapses (FIGS. 14 and 15), sleep disturbances (FIG. 16) and neuronal death (Estes et al., 2011), all of which are prominent features of motor neuron disease. The ability of candidate compounds (at their optimal concentration) can be tested to alleviate individual aspects of TDP-43 phenotypes. In addition to the phenotypic assays described, a splicing assay for TDP-43 based on its known target, the CFTR gene (Buratti et al., 2004), can be developed.

Third, drug cocktails can be utilized. It is possible that sulfonylureas and biguanides have differential rescuing effects on individual TDP-43 phenotypes. A combination therapy can increase the rescuing ability of sulfonylureas or biguanides used separately. Different concentration combinations can be administered and their effect on rescuing TDP-43 toxicity can be determined using the established panel of phenotypic assays.

These experiments can establish what specific aspects of TDP-43 function (e.g., splicing) or motor neuron disease are mitigated by individual drugs and can inform the design of drug cocktails, based on anti-diabetics alone or anti-diabetics combined with other drug categories in the future. 50% or more of the primary compounds validate in secondary screening. Both sulfonylureas and biguanides have shown rescue activity in secondary testing (Table 1, rescue at 25 μM).

It is possible that there will be a limit to the rescuing ability of the candidate drugs because the drugs do not efficiently cross the blood brain barrier, which in Drosophila brains is represented by astrocyte-like surface glia (Stork et al., 2012). It has been shown that Metformin can cross the blood brain barrier (Chen et al., 2009) but it remains unclear whether sulfonylureas have similar properties. A partial rescue can also be due to the fact that individual drugs only mitigate subsets of phenotypes due to TDP-43 overexpression. It is formally possible that some phenotypes will be alleviated but others may be enhanced. Indeed, reports in the literature point to both positive and negative effects of Metformin in Alzheimer's disease (Chen et al., 2009). This underscores the importance of the comprehensive phenotypic panel of assays and can provide insights into combinatorial therapies that target individual phenotypes for an effective rescue. On this point, it has been reported that simultaneous administration of both insulin and Metformin is beneficial in Alzheimer's disease (Chen et al., 2009). These combination therapy experiments can determine whether this can be used as a therapeutic strategy in ALS as well.

It is also possible that the molecular targets of the candidate drugs are poorly conserved, which may limit the drugs' effectiveness. Although that does not seem the case for the candidate drugs, the genetic interaction experiments can determine the role of their known molecular targets (Table 1) in the disease pathology. In addition, the pharmacogenetic approach can determine whether the candidate anti-diabetic drugs work through the predicted molecular targets or have off-target effects.

### b. Identification of Components of the Insulin/PI3K/Akt/TOR Signaling Pathway that Modulate the Neurotoxicity of TDP-43 In Vivo

The insulin pathway has been previously linked to neurodegenerative disorders including ALS (Correia et al., 2012). The findings that three different categories of anti-diabetic drugs with distinct modes of action can mitigate TDP-43's neurotoxicity in vivo indicate that insulin signaling is dysregulated due to TDP-43 overexpression in motor neurons. Results show that the output of insulin signaling is enhanced in the Drosophila model (FIG. 17). Given that the insulin pathway is highly conserved in Drosophila (FIG. 18) and that the fly model recapitulates several hallmarks of ALS pathology, the findings are applicable in mammalian systems.

Western blots can be used to assess the levels of individual components in the insulin pathway in the fly model of ALS. Second, genetic interactions can be used to determine whether loss or gain of function for components of the insulin/PI3K/Akt/TOR signaling cassette modulate known TDP-43 phenotypes (FIGS. 12-16). These experiments can establish critical nodes in the insulin signaling pathway that can serve as targets for future therapeutic development.

To evaluate the levels and activation state of insulin signaling in flies expressing TDP-43 in motor neurons Western blots using antibodies against individual components of the pathway can be performed. For example, antibodies against dILPs, dFoxO, as well as commercially available antibodies against Akt, phosphoAkt and phospho-S6K can be used. Protein samples can be prepared from flies expressing TDP-43 variants in motor neurons and subjected to SDS-PAGE using standard techniques. These results can be compared with the effects of candidate anti-diabetic drugs on individual components of the insulin signaling pathway. This approach can determine which components of the pathway are key to the effect of this therapeutic strategy in vivo and will inform the genetic interaction experiments described herein.

Genetic interaction experiments can be performed. Standard genetic techniques can be used to reduce gene dosage or overexpress components of the insulin signaling pathway in the presence of TDP-43 variants in motor neurons. Gene dosage reduction can be accomplished either using standard loss of function mutations or RNAi approaches. Overexpression can be achieved using “UAS-gene of interest” constructs that can be co-expressed with TDP-43 variants using the D42 Gal4 motor neuron driver. A comprehensive panel of phenotypes, some of which are shown in FIGS. 12-16 and are routine in the laboratory can be performed to assess the suppressing or enhancing effect of insulin signaling components on TDP-43 neurotoxicity in vivo (Estes et al., 2011). These include aggregate formation, motor neuron death, neuromuscular junction (NMJ) morphology and locomotor impairment among others. These experiments can complement the results obtained with Western blots and can establish the ability of individual components to modulate specific aspects of TDP-43 neurotoxicity in vivo.

Western blots, genetic interaction approaches and phenotypic assays are routine in the art. Data shows that TDP-43's neurodegenerative phenotype in the retina is enhanced by co-expression of activated PI3K (via DP110^{CAAX}, its kinase subunit rendered constitutively active by targeting it to the plasma membrane, Leevers et al., 1996) or S6K (S6K^{STDETE}, Barcelo and Stewart, 2002). These findings are supported by an interaction with Akt, whose reduction by RNAi suppresses TDP-43 induced neurodegeneration (FIG. 17). Multiple RNAi lines can be used as well as classical loss of function alleles for each candidate gene of interest.

Altogether these results indicate that insulin signaling is dysregulated due to TDP-43 overexpression in the retina. The fact that biguanides such as Metformin and Phenformin were found to rescue TDP-43 induced lethality is consistent with their established action on AMPK. Indeed, AMPK activation inhibits TOR signaling, which is consistent with the genetic interaction results indicating that TDP-43 overexpression leads to an upregulation of PI3K7Akt/TOR signaling.

The action of sulfonylureas, which is to increase insulin release, appears to be in conflict with the genetic interaction results, which support a hyperactivation of the pathway due to TDP-43 overexpression. Overexpressing dILPs in the context of TDP-43 can determine if dILPs are indeed protective. Also, it is important to note that by administering sulfonylureas, there can be specific benefits to neuronal function and survival independent of insulin/dILP release but rather via neuronal specific Irk3 and Sur expression (Table 1, molecular targets). Immunostainings and Western blots for dILPs can also determine whether they are upregulated upon administering sulfonylureas as is the case in mammalian systems. This apparent paradox of sulfonylureas' neuroprotective effect underscores the importance of the combined approach that can determine the precise phenotypic benefits of the anti-diabetic drugs and of individual components of insulin signaling. It is also possible that feedback loops can be at play and the genetic interaction experiments can uncover such a scenario. Finally, it is possible that sulfonylureas simply improve insulin sensitivity and regulate energy homeostasis by acting on a target different from K_{ATP}. This could occur if K_{ATP} components, Irk3 and/or Sur are not expressed at the right levels or location in the fly, an issue, which can be addressed using the combined pharmacogenetic approach.

The identification of specific components of the insulin signaling pathway that exhibit functional interactions with TDP-43 can provide insights into what aspects of energy homeostasis are dysregulated in ALS and can serve to formulate future hypotheses about TDP-43's mechanisms of disease and therapeutic strategies. Furthermore, the use of multiple TDP-43 variants can identify mutation-specific interactions that can be used for the development of personalized therapeutic intervention.

A major confounding issue in drug discovery is the presence of off-target effects. The fly is an excellent model to address this important issue because candidate drugs can be administered in the context of both TDP-43 as well as gene mutations corresponding to the molecular target of candidate drugs. This combined pharmacogenetic approach avoids the use of additional chemical inhibitors that are traditionally used in vitro to silence molecular targets and tend to have their own off target effects. In addition, the disclosed approach can precisely answer the question of whether a drug acts through its known molecular target or through a different mechanism in vivo, which provides an unparalleled advantage to using the fly model for target validation.

Candidate drugs are administered at the optimal concentrations determined to Drosophila larvae or adults expressing TDP-43 in motor neurons, in the context of genetic alterations (loss or gain function) for the predicted molecular target of individual compounds or various components of the insulin pathway. For example, biguanides can be administered in the context of TDP-43 overexpression and AMPK loss of function mutations. Sulfonylureas can be administered in the context of TDP-43 overexpression and Irk3 and/or Sur loss of function mutations. Western blots can be used to evaluate the effect of these manipulations on the insulin/PI3K/Akt/TOR network.

These experiments can determine whether the candidate anti-diabetic compounds work through predicted components of insulin signaling or alternative targets. If biguanides work indeed through AMPK in the fly model, then AMPK reduction or expressing a non-activatable form of AMPK should block the rescuing effect of these drugs. Similarly, for sulfonylureas, by removing one or both components of K_{ATP}, their neuroprotection can be abolished if they indeed use this channel to rescue TDP-43's phenotypes. Inducible Gal4 lines that, in addition to tissue specificity, also allow for temporal control of gene expression can be used. Thus both TDP-43 and RNAi for AMPK, Irk3 or Sur can be co-expressed at adult stages, which can bypass any developmental requirement of the molecular targets of interest. These data can identify specific segments within the insulin pathway that can play a more important role in modulating the neuroprotective capacity of candidate anti-diabetic compounds.

### c. Identification of New Therapeutic Targets for ALS Through Drug Screening in Drosophila

The Prestwick collection of FDA approved drugs has been used to identify compounds that rescue the adult lethality phenotype due to TDP-43 overexpression in Drosophila motor neurons. The Drosophila model can be used for optimization and secondary validation testing of the candidate PPAR gamma agonists identified in the primary screening. To complement the drug screening approach, genetic interaction experiments can be performed between animals expressing TDP-43 in motor neurons, glia or muscles and mutants in the PPAR gamma and mitoNEET pathways. A battery of phenotypic assays can be used to determine what aspects of TDP-43 neurotoxicity are alleviated by pioglitazone and troglitazone and/or gene mutations and can inform the translational strategies to be performed.

### d. Assay Development and Molecule Validation to Transfer Concept Compound from Fly to Mammalian Systems

Using the extensive medicinal chemistry expertise and available libraries, the active molecules identified herein can be examined and new molecules can be identified that are related either in chemical structure or previously identified biological activity (i.e., chemical or biological “backscreening”). These newly identified molecules can be retested in the Drosophila model to identify improved activities or new chemical series. The Sanofi bioinformatics databases will be available to search and retrieve information pointing to active pathways hit by the active backscreen-derived compounds. Identifying, setting up and performing in vitro cell or biochemical assays that translate the activity found in the whole fly to rapid throughput assays can be performed. These experiments in the mammalian systems can be used to further develop the molecules while also checking back selectively with the fly model to confirm maintenance of the therapeutic phenotype.

### e. In Vivo Drug Screening Identifies Several Classes of Compounds that Rescue TDP-43 Mediated Neurotoxicity

A drug screen aimed at rescuing the adult lethality caused by wild-type or mutant TDP-43 (D169G, G298S, A315T and N345K, FIG. 11) expression in motor neurons has been performed. FDA approved drugs from the Prestwick collection are fed to flies expressing TDP-43 at an initial concentration of 50 μM (as previously described in Chang et al., 2008). This screen has several advantages including: (1) it is performed in vivo, (2) it is based on a robust phenotype (100% adult lethality), (3) it can lead to rapid identification of drugs that are already approved for use in humans and (4) it can inform the future development of new small molecules with enhanced neuroprotective capabilities.

FIG. 11 shows a drug screen for compounds that rescue TDP-43 mediated neurotoxicity in vivo. FDA approved drugs from the Prestwick collection (200 mM in DMSO) were mixed with yeast-cornmeal based food and a bromophenol blue (as a mixing indicator) at a final concentration of 50 μM. Larvae expressing TDP-43 (wt or mutant) were raised on drug containing food. In the absence of drugs, TDP-43 expression was lethal at the pupal/pharate stage. Drugs that rescue TDP-43 induced lethality (anywhere from 1-12 adults) were considered as candidates for future studies.

To date, over 960 compounds have been screened and several classes of drugs have been identified that mitigate TDP-43's neurotoxicity. Pioglitazone and troglitazone, two drugs that target the nuclear receptor PPAR gamma as well as mitochondrial associated protein, mitoNEET (Table 4) were subject to additional studies. These candidate drugs can be used to identify and synthesize new compounds that can be developed into future therapeutics for ALS.

First, new therapeutic targets for ALS can be identified through drug screening. An estimated 75% of human disease genes have homologues in Drosophila. Thus, disease modeling in the fly has led to the identification of important therapeutic leads for several human disorders including Fragile X syndrome (Chang et al., 2008), multiple endocrine neoplasia (Vidal et al., 2005) and Huntington's disease among others (reviewed in Pandey and Nichols, 2011; Rudrapatna et al., 2012). The integration of fly models into unbiased strategies for therapeutic discovery holds great promise due to the high degree of conservation between pathways, the availability of an unparalleled genetic toolbox and relative low costs compared to mammalian systems (reviewed in Pandey and Nichols, 2011). The fly model of ALS based on TDP-43 has all these advantages, exhibits remarkable similarities with the human disease and has already proven fruitful in identifying promising candidate compounds that mitigate TDP-43's neurotoxicity in vivo.

Using the drug screening strategy described in FIG. 11, several candidate compounds have been identified that modulate PPAR gamma and mitoNEET, two molecules involved in cellular metabolism (Table 4). The rather limited rescue, which in many cases is limited to one surviving adult, is likely because 50 μM is not the optimal concentration. Other compounds that have been rescreened have shown lower concentrations (e.g., 30 μM or less) generate more surviving adults.

### f

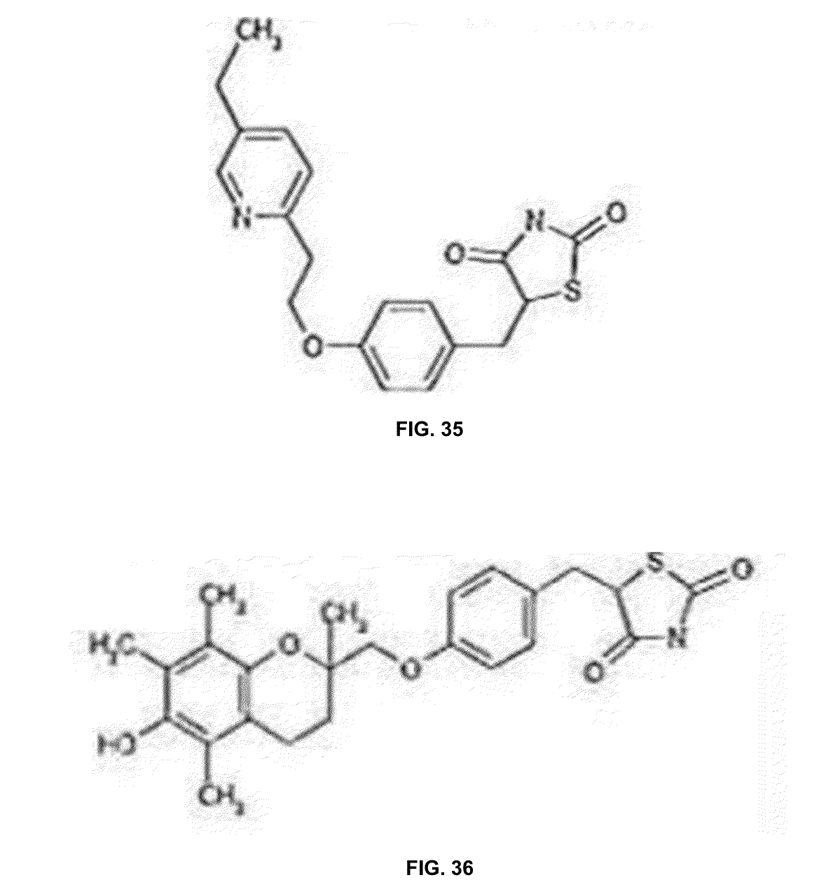

TABLE 4
CANDIDATE COMPOUNDS IDENTIFIED IN PRIMARY
SCREENING IN THE DROSOPHILA MODEL

| Drug name | Pathway | Transgene | Rescue Effect |
|---|---|---|---|
| Pioglitazone | PPAR gamma | wt | 1 adults |
| (FIG. 35) | (lipid/glucose metabolism) | | (out of 15-20 pupae) |
| | mitoNEET (oxidative stress) | | |
| Troglitazone | PPAR gamma | wt | 2 adults |
| (FIG. 36) | (lipid/glucose metabolism) | | (out of 15-20 pupae) |
| | mitoNEET (oxidative stress) | | |

First, to confirm their potential use as therapeutics for ALS, titration experiments can be performed to determine the optimum in vivo concentration of the compounds identified in primary screening. Second, a battery of phenotypic assays, most of which are already established can be used to determine the efficacy of individual compounds in rescuing different aspects of TDP-43 neurotoxicity. Third, genetic interaction experiments can be performed between TDP-43 and mutants in genes predicted to be molecular targets of the candidate compounds and the pathways they belong to. Fourth, additional, new molecules identified in the Sanofi databases can be back-tested in the fly model for their ability to rescue TDP-43 neurotoxicity.

First, the optimization studies. The candidate compounds identified in primary screening (at 50 μM) can be tested for their ability to rescue TDP-43's adult lethality phenotype when delivered at concentrations ranging from 1 to 100 μM in fly food. These experiments are expected to determine an optimal concentration that can generate the highest number of adults rescued upon TDP-43 expression in motor neurons using the D42 Gal4 driver (FIG. 11). A second round of optimization experiments can be conducted to determine additional drug concentrations around the best rescuing values. All experiments can be performed in duplicate. Once optimized, HPLC or mass spectrometry can be used to determine the precise concentration in the brain and the rest of the body.

Second, the secondary testing. A battery of phenotypes due to TDP-43 over-expression in motor neurons in vivo (Estes et al., 2011), in vitro and in glial cells (Estes and Zarnescu) has been established. These include larval locomotor defects, the formation of RNA stress granules/cytoplasmic inclusions, defects in the architecture of neuromuscular junctions and neuronal death, all of which are prominent features of motor neuron disease. The ability of candidate compounds (at their optimal concentration) to alleviate various individual aspects of TDP-43 phenotypes can be tested. In addition to the various phenotypic tests, a splicing assay for TDP-43 based on its known target, the CFTR gene (Buratti et al., 2004), can be developed. These experiments can establish what specific aspects of TDP-43 function (i.e., splicing) or motor neuron disease are mitigated by individual drugs and can inform the design of drug cocktails in the near future.

Third, the genetic interaction experiments. Drosophila harbors 18 nuclear receptor genes, including all six families found in mammals (King-Jones and Thummel, 2005). The closest homologs to vertebrate PPARs are the E75 and E78 loci in the fly, both of which are induced in response to ecdysone, a Drosophila steroid (Thummel and Chory, 2002) can be obtained. Mutants in E75 and E78 can be obtained from the various Drosophila stock centers or individual laboratories. For example, several loss and gain of function alleles are available for both E75 and E78. In vertebrates, PPAR gamma heterodimerizes with retinoid X receptors (RXRs) whose fly homolog, Hr38 (available at lybase.org/reports/FBgn0014859.html) has several loss and gain of function alleles available. These mutants can be crossed to TDP-43 expressing flies and can assess changes in TDP-43's neurotoxicity using the assays described herein. A similar strategy can be used to test for interactions between TDP-43 and the mitoNEET gene (CG1458, flybase.org/reports/FBgn0062442.html).

In addition, publicly available databases where additional information has been curated about specific assays in which the compounds of interest have been identified can be used. For example, troglitazone has also been identified as active in a high-throughput screen for modulators of cytochrome P450. This molecule has several homologs in the fly that can be tested for genetic interactions with TDP-43. A similar strategy can be employed to identify molecular targets of the candidate compounds in chemi- and bio-informatics databases. These genetic interaction experiments can determine which of the tested loci are better molecular targets in the TDP-43 model and can be critical for the design of future small molecules that can specifically interact with the correct target molecule. These experiments underscore the power of the genetic tools available in the fly model and can identify unique molecular targets that can be used for further therapeutic development.

Fourth, the testing of compounds. The fly model can also be used for testing new compounds identified through “backscreening” in the chemical and bioassay databases. These experiments can lead to the identification of new compounds and/or molecular targets. This is important for identifying compounds with a better ability to cross the blood brain barrier or less side effects than the current candidate drugs (Mandrekar-Colucci and Landreth, 2011). Compounds in public databases are placed there because they were active in some previous biological system not necessarily related to the current interest. A chemistry search of the Sanofi database against the structures of some of the most active compounds from the fly screen, including pioglitazone and troglitazone showed that there is a wealth of chemical matter that can be further tested in the fly system.

Medicinal chemists can use proprietary databases to identify an optimal selection of compounds based on the confirmed active “starter compounds” from the Prestwick collection in an effort to improve activity. This class of compounds is notoriously promiscuous in its action and it is possible that targets other than the immediately obvious PPAR gamma are relevant to the rescue effect in the fly. Therefore, in addition, alternative biological targets of the starter compounds (e.g., PPAR gamma and microNEET) can be mined from other databases to add unrelated chemical entities active in the same systems. These new molecules can be selected from several million compounds curated over many years from drug discovery projects in a variety of biological assays or synthesized for their chemical diversity. With this information, particular biological pathways or individual targets in mammalian systems can be focused on to confirm and further improve the chemical matter initially identified. A panel of relevant in vitro mammalian assays can be run with state-of-the art methods and equipment. Many such assays have been already developed. For example, a mouse cell-based PPAR gamma reporter gene assay is available to check that the rank order of compound potencies in fly parallel those in the mouse model.

All of the compositions and/or methods disclosed and claimed herein can be made and executed without undue experimentation in light of the present disclosure. It will be apparent to those skilled in the art that various modifications and variations can be made in the present invention without departing from the scope or spirit of the invention.

More specifically, certain agents which are both chemically and physiologically related can be substituted for the agents described herein while the same or similar results can be achieved. All such similar substitutes and modifications apparent to those skilled in the art are deemed to be within the spirit, scope and concept of the invention as defined by the appended claims.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### g. References for Example 2

The following references, to the extent that they provide exemplary procedural or other details supplementary to those set forth herein, are specifically incorporated herein by reference.
Alexander, M. D., et al. 2002. “True” sporadic ALS associated with a novel SOD-1 mutation. Ann Neurol 52, 680-683.
Banks, G. T., et al. 2008. TDP-43 is a culprit in human neurodegeneration, and not just an innocent bystander. Mamm Genome 19, 299-305.
Beleza-Meireles, A., et al. 2009. Genetic studies of amyotrophic lateral sclerosis: controversies and perspectives. Amyotroph Lateral Scler 10, 1-14.
Buratti, E., et al. 2004. Nuclear factor TDP-43 binds to the polymorphic TG repeats in CFTR intron 8 and causes skipping of exon 9: a functional link with disease penetrance. Am J Hum Genet 74, 1322-1325.
Chang, S., et al. 2008. Identification of small molecules rescuing fragile X syndrome phenotypes in Drosophila. Nat Chem Biol 4, 256-263.
Estes, P. S., et al. 2011. Wild-type and A315T mutant TDP-43 exert differential neurotoxicity in a Drosophila model of ALS. Human molecular genetics 20, 2308-2321.
Freibaum, B. D., et al. 2010. Global analysis of TDP-43 interacting proteins reveals strong association with RNA splicing and translation machinery. Journal of proteome research 9, 1104-1120.
Johnson, B. S., et al. 2009. TDP-43 is intrinsically aggregation-prone and ALS-linked mutations accelerate aggregation and increase toxicity. J Biol Chem.
Jones, J. R., et al. 2005. Deletion of PPARgamma in adipose tissues of mice protects against high fat diet-induced obesity and insulin resistance. Proceedings of the National Academy of Sciences of the United States of America 102, 6207-6212.
Kabashi, E., et al. 2008. TARDBP mutations in individuals with sporadic and familial amyotrophic lateral sclerosis. Nat Genet 40, 572-574.
King-Jones, K., et al. 2005. Nuclear receptors-a perspective from Drosophila. Nature reviews. Genetics 6, 311-323.
Lagier-Tourenne, C, et al. 2009. Rethinking ALS: the FUS about TDP-43. Cell 136, 1001-1004.
Lanson, N. A., Jr., et al. 2011. A Drosophila model of FUS-related neurodegeneration reveals genetic interaction between FUS and TDP-43. Human molecular genetics.
Lessing, D., et al. 2009. Maintaining the brain: insight into human neurodegeneration from Drosophila melanogaster mutants. Nat Rev Genet.
Li, Y., et al. 2010. A Drosophila model for TDP-43 proteinopathy. Proc Natl Acad Sci USA 107, 3169-3174.
Liachko, N. F., et al. 2010. Phosphorylation promotes neurotoxicity in a Caenorhabditis elegans model of TDP-43 proteinopathy. J Neurosci 30, 16208-16219.
Maekawa, S., et al. TDP-43 is consistently co-localized with ubiquitinated inclusions in sporadic and Guam amyotrophic lateral sclerosis but not in familial amyotrophic lateral sclerosis with and without SOD1 mutations. Neuropathology.
Mandrekar-Colucci, S., et al. 2011. Nuclear receptors as therapeutic targets for Alzheimer's disease. Expert Opin Ther Targets 15, 1085-1097.
Neumann, M., et al. 2009. Molecular Neuropathology of TDP-43 Proteinopathies. Int J Mol Sci 10, 232-246.
Neumann, M., et al. 2006. Ubiquitinated TDP-43 in frontotemporal lobar degeneration and amyotrophic lateral sclerosis. Science 314, 130-133.
Paddock, M. L., et al. 2007. MitoNEET is a uniquely folded 2Fe 2S outer mitochondrial membrane protein stabilized by pioglitazone. Proceedings of the National Academy of Sciences of the United States of America 104, 14342-14347.
Pandey, U. B., et al. 2011. Human disease models in Drosophila melanogaster and the role of the fly in therapeutic drug discovery. Pharmacol Rev 63, 411-436.
Polymenidou, M., et al. 2011. Long pre-mRNA depletion and RNA missplicing contribute to neuronal vulnerability from loss of TDP-43. Nature neuroscience 14, 459-468.
Ritson, G. P., et al. 2010. TDP-43 mediates degeneration in a novel Drosophila model of disease caused by mutations in VCP/p97. J Neurosci 30, 7729-7739.
Rosen, D. R. 1993. Mutations in Cu/Zn superoxide dismutase gene are associated with familial amyotrophic lateral sclerosis. Nature 364, 362.
Rudrapatna, V. A., et al. 2012. Drosophila cancer models. Developmental dynamics: an official publication of the American Association of Anatomists 241, 107-118.
Rutherford, N. J., et al. 2008. Novel mutations in TARDBP (TDP-43) in patients with familial amyotrophic lateral sclerosis. PLoS Genet 4, e1000193.
Sephton, C. F., et al. 2011. Identification of Neuronal RNA Targets of TDP-43-containing Ribonucleoprotein Complexes. J Biol Chem 286, 1204-1215.
Sreedharan, J., et al. 2008. TDP-43 mutations in familial and sporadic amyotrophic lateral sclerosis. Science 319, 1668-1672.
Stallings, N. R., et al. 2010. Progressive motor weakness in transgenic mice expressing human TDP-43. Neurobiology of disease 40, 404-414.
Tan, C. F., et al. 2007. TDP-43 immunoreactivity in neuronal inclusions in familial amyotrophic lateral sclerosis with or without SOD1 gene mutation. Acta Neuropathol 113, 535-542.
Thummel, C. S., et al. 2002. Steroid signaling in plants and insects-common themes, different pathways. Genes & development 16, 3113-3129.
Tollervey, J. R., et al. 2011. Characterizing the RNA targets and position-dependent splicing regulation by TDP-43. Nature neuroscience 14, 452-458.
Vidal, M., et al. 2005. ZD6474 suppresses oncogenic RET isoforms in a Drosophila model for type 2 multiple endocrine neoplasia syndromes and papillary thyroid carcinoma. Cancer research 65, 3538-3541.
Wegorzewska, I., et al. 2009. TDP-43 mutant transgenic mice develop features of ALS and frontotemporal lobar degeneration. Proc Natl Acad Sci USA 106, 18809-18814.
Weihl, C. C., et al. TDP-43 accumulation in inclusion body myopathy muscle suggests a common pathogenic mechanism with frontotemporal dementia. J Neurol Neurosurg Psychiatry 79, 1186-1189.
Xu, Y. F., et al. 2010. Wild-type human TDP-43 expression causes TDP-43 phosphorylation, mitochondrial aggregation, motor deficits, and early mortality in transgenic mice. The Journal of neuroscience: the official journal of the Society for Neuroscience 30, 10851-10859.
Zarnescu, D. C, et al. 2005. Come FLY with us: toward understanding fragile X syndrome. Genes Brain Behav 4, 385-392.

### 5. Example 3

The RNA-binding protein TDP-43 has been linked to amyotrophic lateral sclerosis (ALS) both as a causative locus and as a marker of pathology. Loss of function and overexpression studies have shown that alterations in TDP-43 dosage recapitulate hallmark features of ALS pathology, including neuronal loss and locomotor dysfunction. Disclosed herein is a direct in vivo comparison between wild-type and A315T mutant TDP-43 overexpression in Drosophila neurons. When expressed at comparable levels, wild-type TDP-43 exerted more severe effects on neuromuscular junction architecture, viability and motor neuron loss compared with the A315T allele. Some of these differences were compensated by higher levels of A315T expression, which indicated a direct correlation between dosage and neurotoxic phenotypes. Larval locomotion was a parameter that was more affected by the A315T allele than wild-type TDP-43. RNA interference and genetic interaction experiments indicated that TDP-43 overexpression mimicked a loss-of-function phenotype and indicated a dominant-negative effect. Furthermore, neuronal apoptosis did not require the cytoplasmic localization of TDP-43 and neurotoxicity was modulated by the proteasome, the HSP70 chaperone and the apoptosis pathway. The studies disclosed herein examined the phenotypic consequences of the A315T TDP-43 missense mutation.

Amyotrophic lateral sclerosis (ALS) is an adult-onset, progressive neurodegenerative disorder characterized by motor neuron dysfunction, which leads to paralysis and respiratory failure followed by death, which occurs generally within 5 years from diagnosis. About 20% of all ALS patients also exhibit fronto-temporal lobar degeneration, which is characterized by neurodegeneration of the frontal and temporal lobes (1). Approximately 10% of all ALS cases are inherited (familial ALS, fALS) and have been linked to a number of loci, including superoxide dismutase (SOD1), alsin (a GPTase), senataxin (a DNA/RNA helicase), VAMP/synaptobrevin-associated protein B, P150 dynactin, angiogenin, TAR DNA-binding protein (TDP-43) and FUsed in Sarcoma (Fus) (2-8). The remaining 90% of ALS cases are sporadic (sALS) and remain poorly understood.

Extensive pathological studies have identified TDP-43 as a common component of cytoplasmic inclusions found in almost all non-SOD1 cases of ALS studied to date (9-11) as well as in other neurodegenerative disorders. Histological examinations of human tissue obtained at autopsy have defined distinct subtypes of TDP-43-positive cytoplasmic inclusions ranging in shape from filamentous to round aggregates that are present in neurons and sometimes in the surrounding glia (12). TDP-43 gene mutations have been identified in both fALS and sALS patients of diverse ethnicities (3, 11, 13-18).

TDP-43 protein consists of two RNA recognition motifs (RRM1 and 2) as well as a glycine-rich domain within the C terminus (19). In vitro assays have demonstrated that TDP-43 binds with high-affinity UG-rich sequences, consistent with a role in mRNA splicing (20). Except for a single mutation found in the first RNA-binding domain of TDP-43, all other mutations found in ALS patients lie in the C-terminus, including the glycine-rich domain (3, 17, 18). These mutations are amino acid substitutions that are thought to increase TDP-43 phosphorylation and target it for degradation by the proteasome (3). The TDP-43 protein is ubiquitously expressed and co-localizes with Survival of Motor Neuron (SMN) and gemin proteins in the nucleus. Its cellular functions are just beginning to be understood and include transcriptional repression, splicing, miRNA biogenesis, apoptosis and cell division. In cultured neurons, TDP-43 associates with RNA granules and co-purifies with beta-actin and CaMKII mRNAs. Furthermore, TDP-43 co-localizes with fragile X mental retardation protein (FMRP) and Staufen in an activity-dependent manner, suggesting that TDP-43 may regulate synaptic plasticity in vivo by controlling the transport and splicing of synaptic mRNAs (21).

Disclosed herein is the direct comparison of a range of phenotypes produced by expressing wild-type and A315T mutant TDP-43 in the Drosophila nervous system. TDP-43 neurotoxicity was modulated by the proteasome, HSP70 chaperone and apoptotic pathways. Transgenic Drosophila expressing either fly or hTDP-43 variants in two neuronal models, the retina and motor neurons, were generated. TDP-43 expression in photoreceptor neurons led to the formation of cytoplasmic and axonal aggregates in developing retina. Adult eyes expressing TDP-43 variants exhibited progressive neuronal loss and neurodegeneration in a dose-dependent manner. When comparing transgenic lines that express TDP-43 at similar levels, the Drosophila variants were more potent than their human counterparts. Similar differential effects between fly and hTDP-43 as well as between wild-type and the A315T allele were found in motor neurons. Wild-type Drosophila TDP-43 (TBPH) expression in motor neurons led to a relocalization from the nucleus, where it is normally found, to the cytoplasm, where it formed visible aggregates. In contrast, the TBPH A315T mutant as well as the hTDP-43 variants remained restricted to the nucleus regardless of their level of expression (i.e., moderate versus high). For both Drosophila and human variants, wild-type TDP-43 expression was more detrimental than A315T to viability and motor neuron survival, as evidenced by earlier lethality and neuronal apoptosis phenotypes in the nervous system. When expressed at comparable levels with wild-type TBPH, the A315T TBPH variant was restricted to the nucleus and showed modest evidence for neuronal death. These results indicated a direct correlation between the amount of mislocalized (cytoplasmic) TDP-43 and apoptosis in motor neurons, analyses of the hTDP-43 transgenes indicate that cell death can occur in the absence of cytoplasmic aggregation and is likely due to neurotoxic effects caused by excess wild-type TDP-43 or the presence of the A315T allele.

Both the wild-type and A315T alleles of hTDP-43 remain restricted to the nucleus, expression of wild-type hTDP-43 (hwt) resulted in a dramatic loss of motor neurons, but expression of the hA315T mutant led to just a few cells expressing markers of apoptosis. Adult survival and climbing ability also appeared to be more severely affected by overexpression of wild-type TDP-43 than that of the A315T variant. In contrast, larval turning behavior was significantly more affected by expression of A315T than that of wild-type TDP-43. Taken together, wild-type and A315T mutant TDP-43 exerted differential toxicity. The wild-type allele had more dramatic effects on neuronal death, adult survival and climbing ability, whereas the A315T allele had a more pronounced effect on larval turning behavior, which requires complex motor neuron coordination across anterior-posterior and dorsal-ventral axes. In an aspect, there is a dose-dependent effect of TDP-43 expression as evidenced by the direct correlation between TDP-43 protein levels and neurotoxic phenotypes. RNA interference (RNAi) and genetic interaction experiments showed that wild-type and A315T over-expression mimic a loss-of-function phenotype and indicate a dominant-negative effect. Finally, genetic manipulations of proteasome function, the HSP70 chaperone and caspase inhibitors indicate that TDP-43 toxicity is modulated by proteasome-mediated degradation, protein folding, and the apoptosis pathways.

### a. Overexpression of Wild-Type and A315T Mutant TDP-43 in the Eye LED to Neurodegeneration Accompanied by Cell Loss

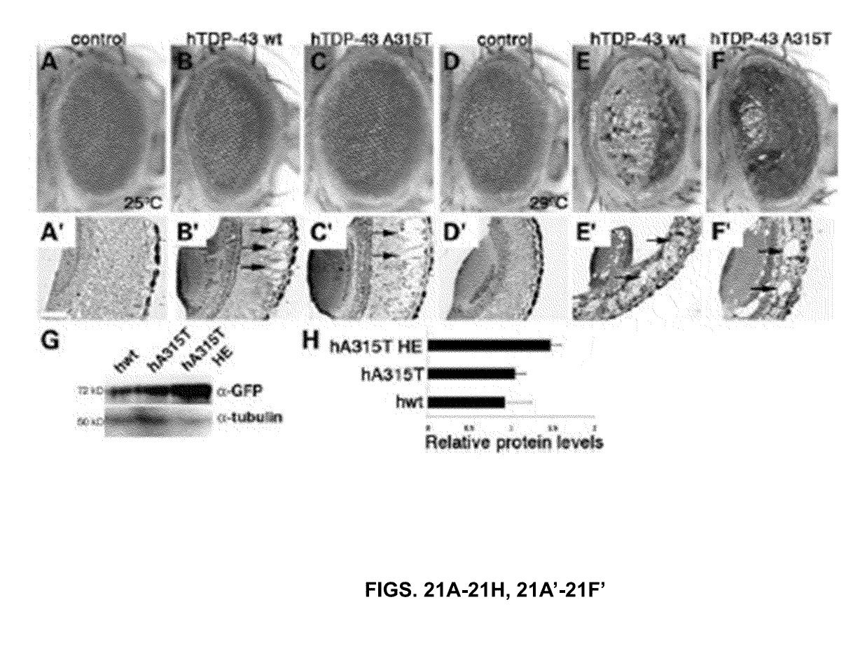

To directly compare the in vivo phenotypes of wild-type TDP-43 with the effects of individual missense mutations found in ALS patients (3), transgenic flies were generated expressing both wild-type and mutant TDP-43 under the control of the UAS promoter. This strategy allowed for tissue-specific expression during Drosophila development using the bipartite Gal4-UAS system (31). Using the GMR Gal4 driver, wild-type and A315T mutant human and TBPH variants were expressed in the developing retina. After surveying several transgenic lines for each TDP-43 variant, wild-type and A315T mutant-expressing lines (one of each) that are comparable in levels of expression were chosen (FIGS. 21G and H for hTDP-43). In addition, a higher expressing A315T line was chosen (hA315T HE, FIGS. 21G and H). As shown in FIG. 21A-C, expression of hTDP-43 (wild-type and A315T mutant) had no visible phenotype in the eye at 25° C. Despite the absence of surface phenotypes, retinal sections through the adult eyes exhibited evidence of cell loss and neurodegeneration (FIG. 21A′-C′), indicating that hTDP-43 expression is toxic in the retina. These results were substantiated by findings that when expressed at higher levels (by raising the flies at 29° C., which increases Gal4 activity), both wild-type and A315T mutant hTDP-43 expression led to visible signs of retinal neurodegeneration (FIG. 21D-F). Plastic sections through these retina showed clear evidence of cell loss (FIG. 21D′-F′). Similar results were obtained with additional hTDP-43 transgenic lines (including hA315T HE), indicating that the milder phenotypes at 25° C. are likely due to the presence of a threshold for hTDP-43 toxicity rather than a positional effect (due to the randomness of trans-gene insertion in the genome). A similar dose response was observed for wild-type TBPH, which led to severe eye neurodegeneration at 25° C. but was 100% lethal when overexpressed at higher levels (by raising the flies at 29° C.). Retinal cell loss had an aging component, as indicated by the finding that hTDP-43-expressing adults (both wild-type and A315T), despite showing little or no visible phenotypes at 25° C. when 1-3 days old (DO) (FIGS. 21B and C), they all exhibited a visible loss of pigmentation at 5 DO. This phenotype worsened as animals age (compare FIG. 21B with 27A and FIG. 21C with 27E).

These results indicated that when expressed at comparable levels, Drosophila and hTDP-43 overexpression had similar consequences, although the fly transgenes appeared to be more potent than their human equivalents. Indeed, flies harbor an additional, previously unreported TDP-43 homolog, namely CG7804, which is 41.6% identical to TBPH and 34.5% identical to hTDP-43. The presence of CG7804 indicate that the wild-type TBPH trans-gene is more toxic than hwt despite being expressed at comparable levels. These results also indicate that TDP-43 and its toxicity in vivo are dose- and age-dependent.

### b. Wild-Type and Mutant TDP-43 Accumulated in Axonal Aggregates in the Developing Eye

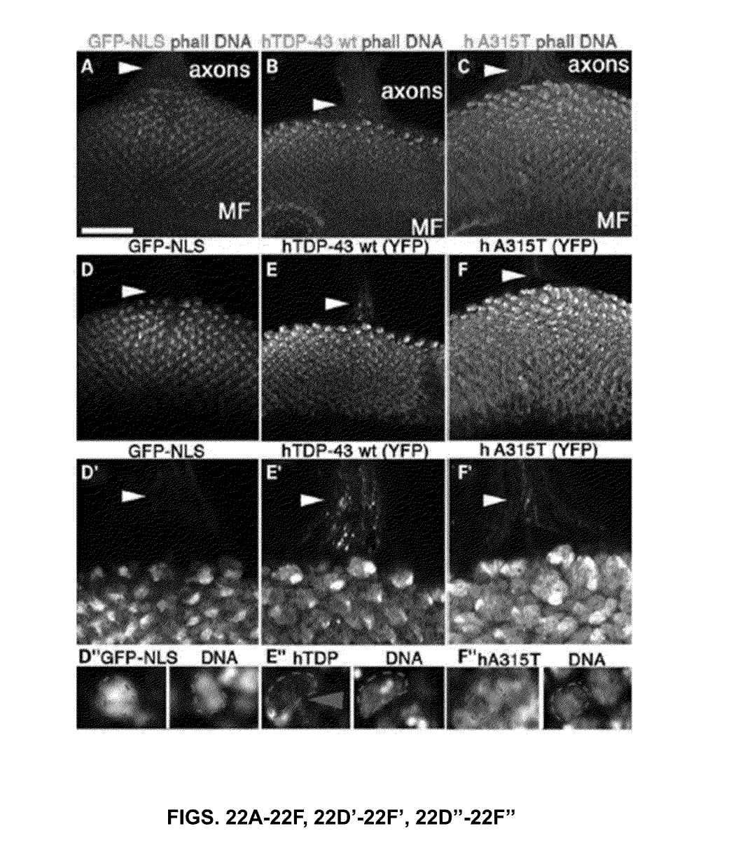

One of the hallmarks of ALS pathology is the accumulation of cytoplasmic inclusions containing TDP-43 (32). Previous reports include both the presence and absence of TDP-43 aggregates are present and absent in models ranging from yeast to flies, zebrafish, cultured neurons and mice (22-24, 27-29, 33, 34). Whether overexpression of wild-type TDP-43 and the A315T mutant transgenes altered the subcellular localization of TDP-43 and lead to the formation of cytoplasmic aggregates was explored. To this end, the distribution of individual, YFP-tagged hTDP-43 transgenes (wild-type and A315T) was compared with nuclear GFP. As seen in FIG. 22, both TDP-43 variants accumulated in axonal aggregates when expressed in developing eye discs (FIG. 22A-F′, arrowheads). When examining their subcellular distribution in comparison with GFP-NLS at high magnification, all TDP-43 variants used in this study exhibited some nuclear as well as some cytoplasmic presence (compare FIGS. 22D″ with E″ and F″). When comparing their subcellular localization, the A315T mutant appeared diffuse (asterisk in FIG. 22F″) in comparison with wild-type TDP-43, which exhibited a more particulate distribution (arrowhead in FIG. 22E″). Similar results were obtained expressing TBPH. These data indicate that all transgenes examined in this study exhibited some level of redistribution from the nucleus into the axons of the developing retina.

### c. Wild-Type TBPH Accumulated in Cytoplasmic Aggregates when Expressed in Motor Neurons

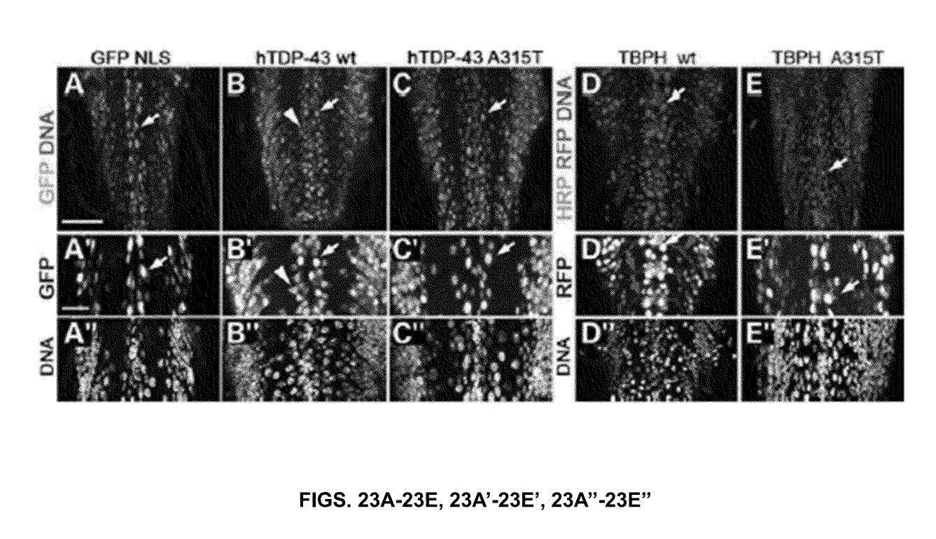

Next, the subcellular distribution of TDP-43 variants in motor neurons was compared. Using the D42 Gal4 driver (35), YFP-tagged wild-type and A315T hTDP-43 were expressed (FIGS. 23B-B″ and 23C-C″, respectively) as well as RFP-tagged, wild-type and A315T TBPH (FIGS. 23D-D″ and 23E-E″, respectively). Then, their localization was compared with that of nuclear GFP (GFP-NLS, FIG. 23A-A″) or RFP (RFP-NLS). These experiments showed that when expressed at similar levels, the hTDP-43 variants remained restricted to the nucleus (compare white arrows in FIG. 23A-C′). Although some cells expressing hwt exhibited a ring-like distribution at the edge of the nucleus (arrowheads in FIGS. 23B and 23B′), hA315T was clearly restricted to the nucleus (arrows in FIGS. 23C and 23C′). A fraction of wild-type TBPH redistributed to the cytoplasm and formed visible aggregates within the neuropil (red arrows in FIG. 23D′), whereas the TBPH A315T allele was mostly restricted to the nucleus (arrows in FIGS. 23E and 23E′). Thus, only wild-type TBPH exhibited a pronounced exit from the nucleus. These data indicate that motor neurons handled expression of Drosophila and human, both wild-type and A315T mutant TDP-43 differently than photoreceptor neurons in which all TDP-43 variants accumulated in axonal aggregates.

### d. Motor Neurons Expressing TDP-43 Variants Exhibited Morphological Defects at the NMJ Synapse

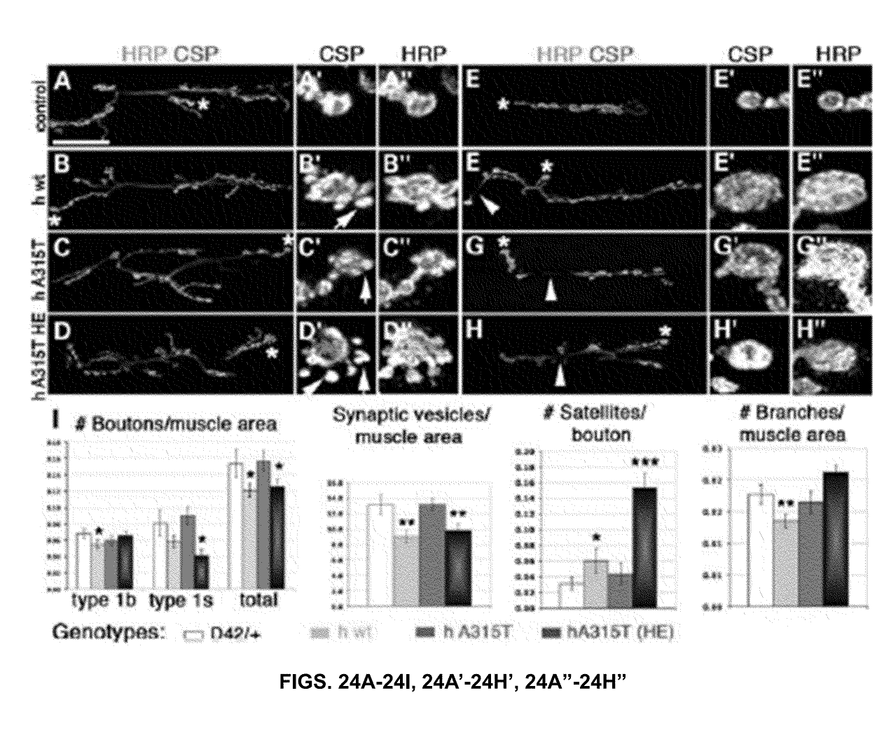

To determine whether TDP-43 overexpression had an impact on the ability of motor neurons to form synaptic connections, the morphology of the larval NMJ was examined (FIG. 24). The larval NMJ synapse at muscles 6/7 consists of structural varicosities referred to as type 1s and type 1b synaptic boutons that form when motor neuron terminals innervate the surface of post-synaptic muscles (36). The pre-synaptic motor neuron membrane at the NMJ was labeled with horseradish peroxidase (HRP) antibodies and the synaptic vesicles within boutons with cysteine string protein (CSP) antibodies (37) (FIGS. 24A-D″ and 24E-H″). Using these markers, the effect of TDP-43 variants on the overall size of the NMJ synapse was measured by quantifying the total number of boutons (as marked by HRP), the area occupied by synaptic vesicles (as indicated by CSP) as well as the number of satellite boutons and axonal branches (using both CSP and HRP labels) (FIG. 24I). First, quantification of the number of type 1s and 1b as well as the total number of boutons (1s plus 1b) per muscle area showed that overexpression of hwt led to a significant decrease in the total number of boutons (FIG. 24I). When equal levels of hA315T protein were expressed at the NMJ, there were no significant changes in bouton numbers (FIG. 24I). However, when higher levels of A315T (hA315T HE; FIGS. 21G and H) were expressed, there was a significant decrease in the number of synaptic boutons at the NMJ, similar to the effect of the lower expressing human wild-type TDP-43 line (FIG. 24I).

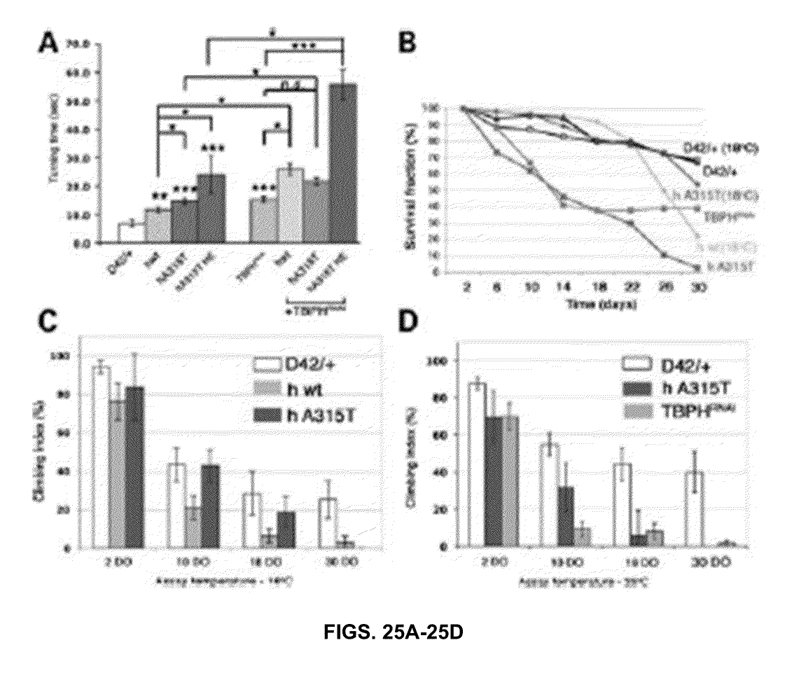

The total area occupied by synaptic vesicles were quantified and it was found that hwt leads to a significant decrease compared with controls (FIG. 24I). In addition, the morphological analyses revealed the presence of supranumerary satellite boutons (arrow in FIG. 24B′) due to expression of hwt. These structures represent abnormal synaptic growths and indicate potential defects in microtubule organization and intracellular trafficking. As with the total number of boutons, comparable levels of hA315T had no effect, but when the higher hA315T-expressing line (hA315T HE) was used, the area occupied by synaptic vesicles was decreased and the number of satellite boutons was increased, similar to the effect of hwt (FIG. 24I). These results indicate that TDP-43 can control synaptic function at the NMJ (FIG. 25A). Additional quantifications included the number of axonal branches per muscle area (FIG. 24I). hwt but not hA315T or hA315T HE led to a significant decrease in the number of axonal branches at the NMJ (FIG. 24I).

Also investigated was whether TDP-43-overexpressing larvae exhibited signs of motor neuron degeneration as reported in ALS patients. Neurodegeneration at the larval NMJ has been shown to manifest through the presence of synaptic ‘footprints’, i.e., structural remains of boutons that are positive for post-synaptic Dlg but lack presynaptic markers such as CSP and HRP (38). Because no obvious ‘footprints’ were found in the larval anterior segment A3, the more posterior segment A6 was also examined, which is thought to be more sensitive to motor neuron degeneration, presumably due to the increased length of the motor neuron axons. Although some thinning of the neuronal membrane (as indicated by HRP staining) was observed with all TDP-43 variants (arrowheads, FIG. 24E-H, no terminal boutons positive for Dlg but showing reduced CSP staining were found. Experiments using fly transgenes also showed that when comparable levels of wild-type and A315T mutant TBPH are expressed in motor neurons, the wild-type allele has a more pronounced effect on the size of the NMJ (as indicated by its effect on the total number of boutons per muscle area) and the number of satellite boutons. These data indicate that TDP-43 regulated NMJ morphology and that despite some differences between the effects of Drosophila and hTDP-43, the wild-type allele has more pronounced phenotypic effects at the NMJ than the A315T variant when expressed at similar levels. These results indicated that by expressing it at higher levels, hA315T generated toxicity comparable with that of hwt in regard to bouton numbers, synaptic vesicle area, and satellite boutons (FIG. 241).

### e. Locomotor Activity, Viability, and Survival were Impaired Regardless of the Presence of Detectable Tdp-43 Cytoplasmic Aggregates

The defects detected at the NMJ, which included the decrease in the area occupied by synaptic vesicles and the increased number of satellite boutons, indicated that overexpression of TDP-43 variants can lead to locomotor defects. As such, locomotor ability was assayed using larval turning assays (39). Crawling third instar larvae were gently rolled ventral side up, and the time needed to turn back to dorsal side up was recorded. As seen in FIG. 25A, both hwt and the hA315T allele led to a significant increase in the time needed to turn over and resume crawling on the ventral side. In this assay, the A315T mutant phenotype was significantly worse than that of hwt. The hA315T HE transgene leads to a higher level of impairment in larval motor coordination as measured by larval turning assays (FIG. 25A). Upon comparison with the turning behavior of TBPH RNAi in motor neurons (D42::TBPH^{RNAi}), overexpression of both hTDP-43 variants mimicked a loss-of-function phenotype (FIG. 25A). Similar results were obtained with the TBPH trans-genes, except that no significant differences were noted between the effect of wild-type and A315T.

To determine whether the TDP-43 variants act as loss- or gain-of function mutants, the hTDP-43 variants were coexpressed together with TBPH RNAi (FIG. 25A). Coexpression resulted in a significant downregulation of the endogenous Drosophila TBPH expression (note that TBPH^{RNAi} targets both Drosophila homologs, TBPH and CG7804). These experiments showed that the reduction of TBPH by RNAi enhanced the toxic effect of both wild-type and A315T hTDP-43 expression in motor neurons and indicated that hTDP-43 overexpression in motor neurons can act as a dominant negative, at least in regard to larval turning behavior.

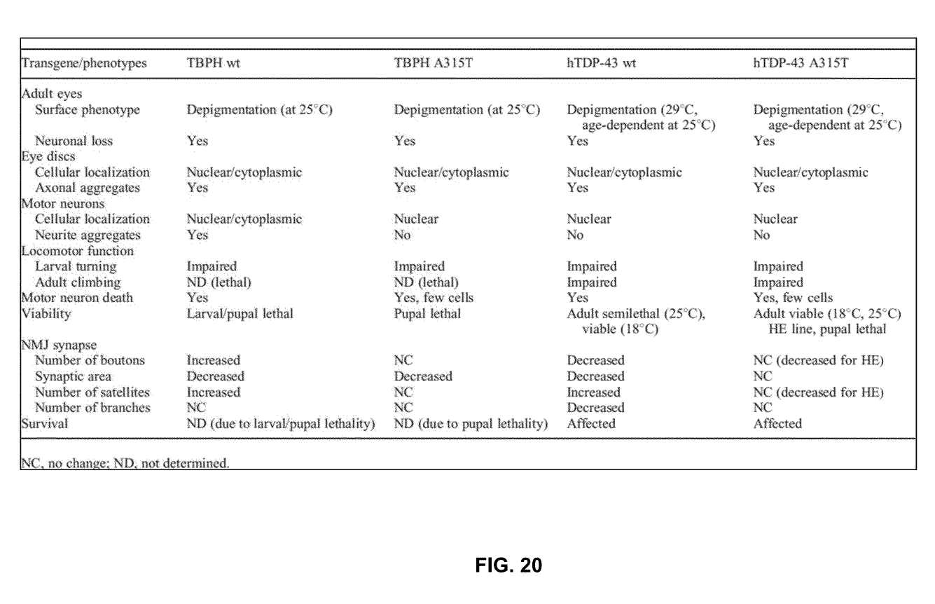

Expression of TDP-43 during development had dramatic effects on the viability of the fly. Wild-type and A315T TBPH expression in motor neurons leads to lethality at both the larval and pupal stages. Comparable levels of wild-type and A315T hTDP-43 were less toxic than those of Drosophila counterparts and exerted differential effects on viability and survival when expressed in motor neurons (FIG. 20 and FIG. 25B). Expression of hwt was semilethal at 25° C., but adults were viable at 18° C. When expressed at comparable levels, the hTDP-43 A315T mutant had no detectable effect on viability (adults were obtained at both 18 and 25° C.). Higher levels of A315T, namely hA315T HE expression, in motor neurons resulted in 100% pupal lethality at 25° C. Although viable adults were obtained with both wild-type and A315T mutant hTDP-43 expression, an effect on survival was observed. As seen in FIG. 25B, control flies exhibited a 30% decrease in survival 30 days after eclosion (at both 18° C. and 25° C.). Expression of hwt at 18° C. led to a 78% decrease in survival, whereas the A315T-expressing adults exhibited a 47% reduction in survival at 18° C. and 98% at 25° C. Because only few adults expressing hwt were obtained at 25° C., both survival and adult locomotor assays with the wild-type transgene were performed at 18° C. only (FIGS. 25B and C).

Next, the effects of wild-type and A315T mutant hTDP-43 on adult locomotor function were tested. Using climbing assays (40), it was found that at 18° C., control flies exhibited a progressive decline in their ability to climb (94.3% for 2 DO to 25.4% for 30 DO flies) (FIG. 25C), whereas expression of hTDP-43 variants had a more significant impact. hwt-expressing adults showed a decline in climbing ability from 76.3% at 2 DO to 3.1% at 30 DO. The A315T-expressing adults were comparable with controls except for the last time point (83.7% at 2 DO to 0% at 30 DO). At 25° C., the A315T allele showed a progressive and significant decline in climbing ability compared with controls (FIG. 25D). The impairment in locomotor function due to TDP-43 overexpression mimicked the effect of TDP-43 loss (using TBPH RNAi; FIG. 25D).

These results indicated that when expressed at comparable levels, hwt was more detrimental to viability, survival and adult climbing than the A315T allele. As with the larval turning results, these effects mimicked the phenotypes due to TDP-43 loss of function in motor neurons, indicating that TDP-43 overexpression can also act as a dominant negative in regard to survival and motor neuron function. hA315T was more detrimental than hwt for larval turning but not adult climbing behavior. These findings indicated that certain behaviors requiree more complex integration of neuromuscular function and were more sensitive to the A315T mutation than other phenotypes. These results indicated that A315T impaired neuromuscular function through a different mechanism than that of wild-type TDP-43.

### f. Cytoplasmic Aggregation of TDP-43 was not Required for Apoptotic Death in Motor Neurons

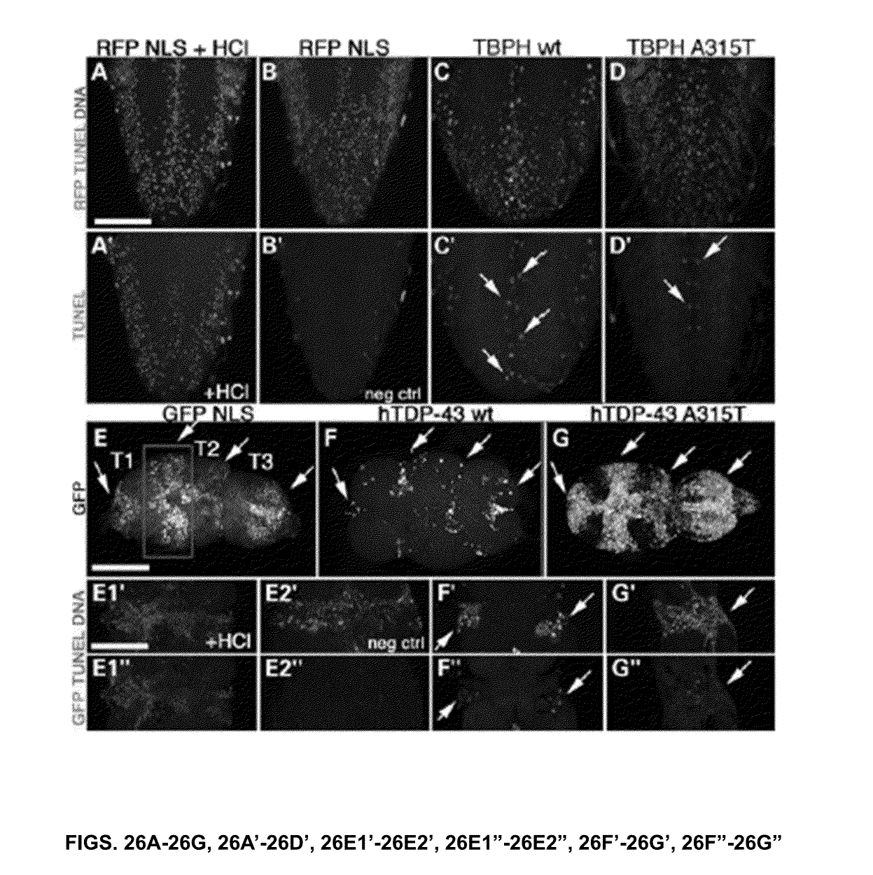

A hallmark of ALS pathology is motor neuron dysfunction accompanied by apoptotic death (41). Although in the retina there is clear evidence for cell loss (FIG. 21), transgenes also affect motor neuronal survival. Terminal dUTP nick end labeling (TUNEL) assays were performed in the larval ventral ganglia, where motor neurons reside. FIG. 26 shows that apoptosis was detected in several cells that coincide with wild-type TBPH-expressing motor neurons [compare FIGS. 26C and 26C′ with 26A and 26A′ (positive control) and 26B and 26B′ (negative control)]. Apoptotic death due to A315T TBPH expression in motor neurons was very modest (FIGS. 26D and 26D′). The human transgenes expressing wild-type and A315T mutant hTDP-43 at comparable levels did not show any evidence of apoptosis in the larval ventral ganglia. The larvae expressing hA315T HE contained a few TUNEL-positive cells and died as pupae.

Given that the hTDP-43 variant expression produced viable adults and that TBPH variants were lethal at earlier stages, cell death in the animals expressing hTDP-43 at comparable levels occurred later in development, i.e., in the adult nervous system. TUNEL assays in adult thoracic ganglia expressing either wild-type or A315T mutant hTDP-43 were performed. Indeed, as shown in FIG. 26F-F″, hwt expression resulted in a dramatic loss of motor neurons as evidenced by: (i) a visible reduction in the number of cells expressing the hTDP-43 transgene (FIG. 26F, compare with 26E) and (ii) the presence of TUNEL-positive cells that coincided with transgene expression (compare FIGS. 26F′ and F″ with controls in FIG. 26E1′-E2″). No obvious signs of cell loss were present in the context of hA315T expression (FIG. 26G, compare with 26E). TUNEL assays indicated the presence of a few apoptotic cells that coincide with hA315T-expressing cells (FIGS. 26G′ and 26G″). These results indicated that regardless of its subcellular localization (nuclear or cytoplasmic), TDP-43 overexpression lead to motor neuron death by apoptosis. These data also indicate that wild-type TDP-43 exerted higher toxicity than the A315T variant (FIG. 20).

### g. TDP-43 Toxicity was Modulated by the Proteasome and the HSP70 Chaperone Activities as Well as the Apoptosis Pathway

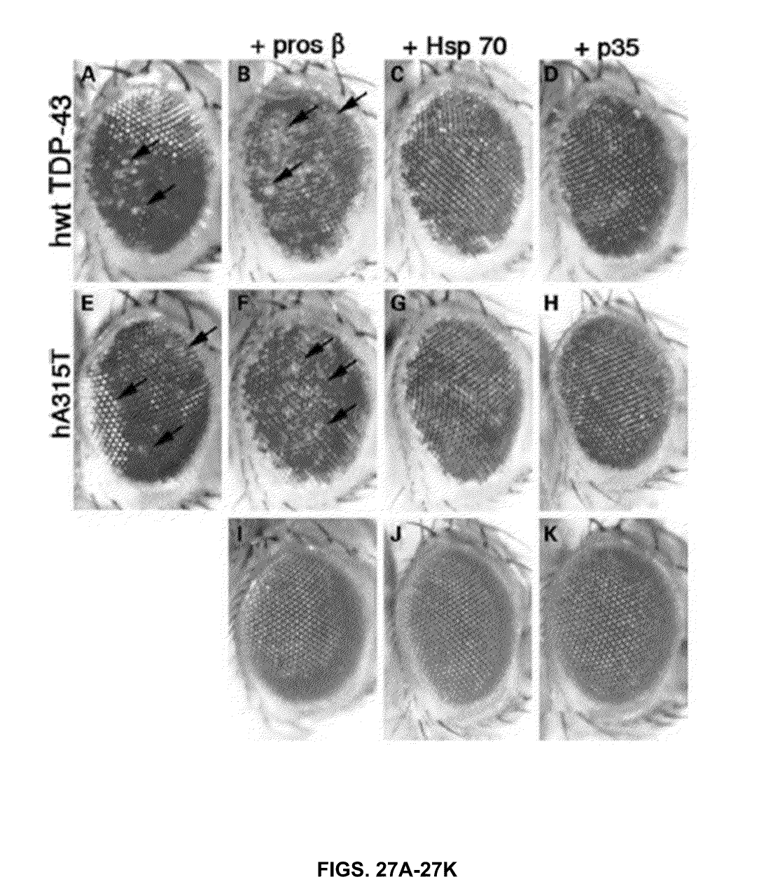

TDP-43 toxicity includes hyperphosphorylation of the mutated variants, followed by excessive targeting to the proteasome, which can become functionally overwhelmed (3, 30). To test whether the proteasome contributes to TDP-43 neurotoxicity in vivo, a dominant-negative form of the 132 subunit of the 20S proteasome complex—namely prosβ (42), was used. Overexpression of prosβ alone in the eye did not produce a visible phenotype (FIG. 27I), However, when coexpressed with wild-type and A315T mutant hTDP-43, it enhanced the depigmentation phenotype due to TDP-43 overexpression (FIGS. 27A and 27E with 27B and 27F, respectively). Although this enhancement was observed throughout adult life, the data shown were from 15-day-old adults when the hTDP-43 phenotypes are more clearly visible at 25° C.

Whether overexpression of the HSP70 chaperone rescued the eye phenotypes due to wild-type and A315T overexpression was tested. HSP70 was chosen due to its ability to rescue the toxicity of polyglutamine, α-synuclein and RNA-mediated neurodegeneration in Drosophila models, in some instances by alleviating the presence of protein aggregates (43-45). As seen in FIG. 27, over-expression of human HSP70 alleviated the depigmentation phenotype due to hTDP-43 (wild-type and A315T) expression in the eye (compare FIGS. 27C and 27G with 27A and 27E, respectively). These data indicated that TDP-43 toxicity can be due to aggregates that pose an overwhelming stress on the proteasome but can be mitigated by excess HSP70 chaperone activity.

Inhibition of apoptosis can mitigate the eye phenotypes due to wild-type and A315T overexpression. As seen in FIGS. 27D and 27H (compare with FIGS. 27A and 27E, respectively), overexpression of the p35 baculovirus protein, a caspase inhibitor (46), rescued the TDP-43 phenotypes in the adult eye. These results indicated that caspase-mediated apoptotic pathways mediated in part the toxicity due to TDP-43 overexpression in the eye.

### h. Summary of Results for Example 3

To uncover the consequences of TDP-43 mutations linked to ALS, a direct in vivo comparison of the neuronal and functional phenotypes resulting from overexpression of wild-type and the A315T mutant TDP-43 in the Drosophila nervous system was performed. Expression of either allele (wild-type or A315T) of TDP-43 in photoreceptor and motor neurons led to effects on viability, locomotor function and survival. In the developing retina, overexpression of the TDP-43 variants resulted in the formation of cytoplasmic and axonal aggregates accompanied by neuronal loss. The eye tissue showed the highest sensitivity to overexpression of TDP-43 and revealed the most dramatic alterations in the subcellular localization of TDP-43, resembling those found in postmortem ALS tissues (9, 11). When TDP-43 variants were expressed in motor neurons, several hallmark features of ALS occurred, including cytoplasmic aggregates, cell death, and defects in locomotor ability (FIG. 20). When expressed at comparable levels, wild-type TDP-43 was more toxic than the A315T mutant in regard to viability, survival, NMJ anatomy and adult climbing behavior but not for larval turning behavior.

By simply increasing expression of the mutant allele several, although not all, phenotypes resulting from overexpression of wild-type TDP-43 were recapitulated. These include effects on viability. Select NMJ features such as decreased synaptic area and malformed synaptic boutons that may be more sensitive to mutations in TDP-43 than others (e.g., axon branching). Thus, these studies show that TDP-43 was inherently toxic. These studies also show that wild-type and A315T mutant TDP-43 exerted differential toxicity when evaluated by a repertoire of cellular and behavioral assays. When expressed at comparable levels, the TBPH variants were more toxic than their human counterparts. These studies show that TDP-43 was required for the proper morphology of the NMJ synapse. The morphological analyses described herein revealed novel phenotypes, including the reduced area occupied by synaptic vesicles, which appeared to be most sensitive to alterations in TDP-43 (both wild-type and A315T) and indicated the presence of functional defects at the synapse. Indeed, larval turning assays, which rely on complex motor coordination along the anterior-posterior and dorsal-ventral axes of the larvae, showed that both the wild-type and A315T alleles affected locomotor ability. Larval turning was the only assay in which the A315T mutant showed higher toxicity than the wild-type allele. Additional evidence for TDP-43's effect on locomotor function was provided by the adult climbing assays. These results indicated that TDP-43 overexpression affected synaptic function.

These results indicate that cytoplasmic aggregates were not required for motor neuron death. Upon wild-type TBPH overexpression, motor neuron death was sparsely detected in larval ventral ganglia before lethality ensued. When hwt was overexpressed, adult motor neuron numbers were significantly reduced in thoracic ganglia and we found strong evidence for apoptotic death. In contrast, overexpression of A315T TBPH led to barely detectable apoptosis, and hA315T resulted in some cells positive for TUNEL staining Apoptosis detection was also marginal in the ventral ganglia of the higher expressing hA315T HE larvae.

The genetic interactions with the proteasome indicated that the insoluble TDP-43 products that arise from overexpression or the presence of missense mutations (3, 23, 30, 53) may normally be processed by the proteasome degradation machinery. By impairing the function of the proteasome, insoluble TDP-43 products cannot be cleared efficiently, which accounted for the enhanced toxicity observed in the eye. These results provided an in vivo demonstration for the involvement of the proteasome in TDP-43 neurotoxicity. Given the dramatic cell loss evident in the retina, inhibition of caspases through overexpression of the p35 baculovirus protein also alleviated the phenotype of TDP-43 overexpression in the eye.

In summary, these studies provide evidence that wild-type and mutant TDP-43 overexpression in Drosophila recapitulated hallmark aspects of ALS pathology. When expressed at comparable levels, wild-type TDP-43 exerted higher toxicity than the A315T allele, in regard to viability, survival, neuronal loss, and adult locomotor function but not larval locomotor behavior.

### 6. Experimental Design for Example 3

### A. Drosophila Genetics

All Drosophila stocks and crosses were kept on standard yeast/cornmeal/molasses food at 25° C. unless otherwise noted. TBPH cDNA GH09868 was obtained from the Drosophila Genome Project in the pOT2 vector. Following PCR amplification and site-directed mutagenesis (QuikChange II, Stratagene), the inserts (wild-type and A315T) were cloned into the pUAST destination vectors pTGW and pTRW (pUAST with N-terminal GFP and RFP, respectively; Drosophila Genome Resource Center), using the Gateway Technology (Invitrogen). hTDP-43, wild-type and A315T with YFP C-terminal tag (in pRS416 yeast expression vector, from A. Gitler) were cloned into the NotI and KpnI sites of the pUAST germline transformation vector. Transgenic lines were mapped and balanced using standard genetic techniques and include GFP TBPH wild-type, RFP TBPH wild-type and A315T, hTDP-43 wild-type YFP, hTDP-43 A315T YFP. Gal4 drivers used in this study include the eye-specific GMR Gal4 R13 and the motor neuron driver D42 Gal4 (35). TBPH RNAi lines were obtained from the Vienna Drosophila RNAi Center (lines w1118; P{GD6943}v38377 and w1118; P{GD6943}v38377) and used to generate a double-RNAi recombinant stock. GMR Gal4 wild-type hTDP-43 and GMR Gal4 hA315T stocks were generated using standard meiotic recombination techniques. For genetic interactions, the following stocks were used: (i) w1118; P{w[+mC]=UAS-Prosbeta2[1]}1B (dominant-negative form of the 20S proteasome core subunit); (ii) w1118; P{w[+mC]=UAS-Hsap\HSPA1L.W}53.1/CyO (human molecular chaperone Hsp70) and (iii) w[*]; P[w[+mC]=UAS-p35.H}BH2 (baculo-virus-derived apoptosis inhibitor).

### b. Western Blots

To determine the relative expression levels of the various TDP-43 transgenes used in this study, Drosophila heads were collected from adults expressing wild-type and A315T mutant, Drosophila or hTDP-43, using the GMR Gal4 driver. Following homogenization in 2× Laemmli buffer, the lysates were resolved on SDS-PAGE and then transferred to a PVDF membrane (Millipore). Drosophila and hTDP-43 transgenes were detected using rabbit polyclonal anti-GFP (Invitrogen) at 1/3000, rabbit polyclonal anti-TARDBP (Abcam) at 1/1250 or rabbit polyclonal anti-TBPH at 1/3000. Tubulin was used as a loading control and was detected using a mouse monoclonal anti-tubulin antibody (Millipore) at 1/1000. The secondary antibody used was goat anti-mouse- or goat anti-rabbit-conjugated HRP, as appropriate, at 1/1000 (Thermo Scientific). Proteins of interest were visualized using SuperSignal West Femto Substrate (Thermo Scientific). Protein levels were quantified using NIH Image software.

### c. Immunohistochemistry and Imaging

Adult fly eyes were imaged with a Leica MZ6 microscope equipped with an Olympus DP71 camera and controlled by Olympus DP Controller and Olympus DP Manager software. Individual images were processed using Adobe Photoshop CS2 (Adobe).

Larval NMJ preparations have been described previously (55). Briefly, wandering third instar larvae were filleted, pinned out on Sylgard dishes, fixed in 3.5% formaldehyde in PBS, pH 7.2, for 20 min and then permeabilized with 0.1% Triton X-100. Following treatment in a blocking agent consisting of 2% BSA and 5% NGS, the fillets were stained with anti-DCSP2 at 1/300 (DSHB), anti-HRP-FITC at 1/50 (Sigma) and anti-Dlg polyclonal at 1/900 (gift of Peter Bryant). Secondary antibodies at 1/1000 were from Molecular Probes. Larval muscles 6 and 7 were imaged in abdominal segments A3 and A6 on a Nikon PCM 2000 confocal microscope and were displayed as a projection of 1 mm serial sections. Type 1b, 1s, and satellite boutons as well as branches were manually counted and total CSP area was determined using Metamorph Image Analysis software (Universal Imaging). All measurements were divided by total muscle area to take into account variations in the size of the individual larvae.

Eye discs and larval ventral ganglia were dissected and prepared as above. Eye discs were stained with either rhodamine phalloidin (1/300), FITC phalloidin (1/150; Sigma) or Alexa 647 phalloidin (1/300) (Molecular Probes) as well as Hoechst 33342 (Invitrogen) at 1/10 000 and, if appropriate, anti-GFP-FITC (Rockland) at 1/200. Ventral ganglia were stained as above except that instead of phalloidin, they were stained with anti-HRP-FITC or TRITC (Sigma) at 1/50. They were imaged on a Zeiss Meta 510 confocal microscope and displayed as projections of 1 mm serial Z sections.

### d. Eye Sections

Adult eyes were embedded and sectioned as described previously (51). In brief, following the removal of the proboscis, adult heads were fixed in Trump's fixative (4% paraformaldehyde, 1% glutaraldehyde, 100 mM cacodylate buffer, pH 7.2, 2 mM sucrose, 0.5 mM EGTA) overnight. The following day, the samples were washed three times in 100 mM cacodylate buffer with 264 mM sucrose, then placed in a 0.5% 0.04 in 100 mM cacodylate buffer for 1 h and rinsed for 10 min in 100 mM cacodylate buffer with 264 mM sucrose. Following a dehydration protocol using ethanol and propylene oxide, samples were embedded in Embed 812 and baked at 65° C. overnight. Using a Reichert Jung Ultracut E Ultramicrotome, 1 mm thick plastic sections were cut and stained with 1% toluidine blue in 1% sodium borate. Images were acquired using a Zeiss Axioplan microscope equipped with an Olympus DP71 camera and controlled by Olympus DP Controller and Olympus DP Manager software.

### e. TUNEL Assay

TUNEL assays were performed on larval ventral and adult thoracic ganglia, using the In Situ Cell Death Detection Kit (Roche, Indianapolis, Ind., USA). Briefly, ganglia were fixed in semi-intact preparations with 3.5% formaldehyde in PBS, pH 7.2, for 20 min. After permeabilization with 0.05% Triton X-100, the ganglia were treated with 10 μg/ml proteinase K (Fermentas) for 10 min at 37° C. The ganglia were then dissected into microtiter dishes. The positive control for the TUNEL reaction consisted of ganglia treated with 2 N HCl for 30 min. The TUNEL reaction was carried out for 1.5 h at 37° C. as per manufacturer's instructions using, as appropriate, a TMR-Red or an FITC label. Ganglia were then stained with Hoechst 33342 (Invitrogen) at 1/10 000 and, if appropriate, anti-GFP-FITC (Rockland) at 1/200. Preps were mounted in 4% N-propyl gallate in 95% glycerol and imaged on a Zeiss Meta 510 confocal microscope. Ganglia were displayed as projections of 1 mm serial Z sections.

### f. Locomotor Function Assays

### (1) Larval Turning Assays

Third instar wandering larvae were placed on a grape juice plate at room temperature. After becoming acclimated, crawling larvae were gently turned onto their backs (ventral side up) and monitored until they were able to turn back (dorsal side up) and continue their forward movement. The amount of time that it took each larva to complete this task was recorded. Three to five trials of 8 to 10 larvae were performed for each genotype. Student's t-test was performed to assess statistical significance.

### (2) Climbing Assays

Expression of the Drosophila transgenes using D42 Gal 4 was lethal before adulthood, primarily at the pupal stage. Therefore, climbing assays were performed with adults expressing wild-type and A315T mutant hTDP-43 in motor neurons. Owing to effects on viability from these transgenes as well, flies were raised at both 25 and 18° C. Ten (1-day-old) adult males of each genotype were collected and tested for their climbing ability starting when 2 DO and every 4 days thereafter, until they reached 30 days after eclosion. Five to ten such cohorts (50-100 males) were tested for each genotype. To assess their climbing ability, the flies were transferred to an empty vial marked at 5 cm from the bottom (40). After being allowed to acclimate to the new environment (˜30 s), flies were gently tapped down to the bottom of the vial and then the time it took each fly to pass the 5 cm mark was recorded. All flies that climbed the 5 cm up the vial in 18 s or less passed, whereas those that could not climb that high or took longer than 18 s failed. Both the number of flies that passed and the number of flies surviving were recorded each time the test was performed. The climbing index for each genotype was calculated as the number of flies that passed the climbing test, normalized to the number of survivors on the day of the test. Survivability was calculated by dividing the number of flies alive on each day by the number alive on day 2.

### G. References for Example 3

1. Banks, G. T., et al. (2008) TDP-43 is a culprit in human neurodegeneration, and not just an innocent bystander. Mamm. Genome, 19, 299-305.
2. Rosen, D. R., et al. (1993) Mutations in Cu/Zn superoxide dismutase gene are associated with familial amyotrophic lateral sclerosis. Nature, 362, 59-62.
3. Kabashi, E., et al. (2008) TARDBP mutations in individuals with sporadic and familial amyotrophic lateral sclerosis. Nat. Genet., 40, 572-574.
4. Beleza-Meireles, A., et al. (2009) Genetic studies of amyotrophic lateral sclerosis: controversies and perspectives. Amyotroph. Lateral Scler., 10, 1-14.
5. Vance, C., et al. (2009) Mutations in FUS, an RNA processing protein, cause familial amyotrophic lateral sclerosis type 6. Science, 323, 1208-1211.
6. Valdmanis, P. N., et al. (2009) Recent advances in the genetics of amyotrophic lateral sclerosis. Curr. Neurol. Neurosci. Rep., 9, 198-205.
7. Lagier-Tourenne, C., et al. (2009) Rethinking ALS: the FUS about TDP-43. Cell, 136, 1001-1004.
8. Kwiatkowski, T. J. Jr., et al. (2009) Mutations in the FUS/TLS gene on chromosome 16 cause familial amyotrophic lateral sclerosis. Science, 323, 1205-1208.
9. Maekawa, S., et al. (2009) TDP-43 is consistently co-localized with ubiquitinated inclusions in sporadic and Guam amyotrophic lateral sclerosis but not in familial amyotrophic lateral sclerosis with and without SOD1 mutations. Neuropathology, 29, 672-683.
10. Tan, C. F., et al. (2007) TDP-43 immunoreactivity in neuronal inclusions in familial amyotrophic lateral sclerosis with or without SOD1 gene mutation. Acta Neuropathol., 113, 535-542.
11. Van Deerlin, V. M., et al. (2008) TARDBP mutations in amyotrophic lateral sclerosis with TDP-43 neuropathology: a genetic and histopathological analysis. Lancet Neurol., 7, 409-416.
12. Neumann, M., et al. (2006) Ubiquitinated TDP-43 in frontotemporal lobar degeneration and amyotrophic lateral sclerosis. Science, 314, 130-133.
13. Daoud, H., (2009) Contribution of TARDBP mutations to sporadic amyotrophic lateral sclerosis. J. Med. Genet., 46, 112-114.
14. Gitcho, M. A., et al. (2008) TDP-43 A315T mutation in familial motor neuron disease. Ann. Neurol., 63, 535-538.
15. Kuhnlein, P., et al. (2008) Two German kindreds with familial amyotrophic lateral sclerosis due to TARDBP mutations. Arch. Neurol., 65, 1185-1189.
16. Pamphlett, R., et al. (2009) TDP-43 neuropathology is similar in sporadic amyotrophic lateral sclerosis with or without TDP-43 mutations. Neuropathol. Appl. Neurobiol., 35, 222-225. Human Molecular Genetics, 2011, Vol. 20, No. 12 2321
17. Rutherford, N. J., et al. (2008) Novel mutations in TARDBP (TDP-43) in patients with familial amyotrophic lateral sclerosis. PLoS Genet., 4, e1000193.
18. Sreedharan, J., et al. (2008) TDP-43 mutations in familial and sporadic amyotrophic lateral sclerosis. Science, 319, 1668-1672.
19. Johnson, B. S., et al. (2008) A yeast TDP-43 proteinopathy model: exploring the molecular determinants of TDP-43 aggregation and cellular toxicity. Proc. Natl Acad. Sci. USA, 105, 6439-6444.
20. Buratti, E., et al. (2001) Characterization and functional implications of the RNA binding properties of nuclear factor TDP-43, a novel splicing regulator of CFTR exon 9. J. Biol. Chem., 276, 36337-36343.
21. Wang, I. F., et al. (2008) TDP-43, the signature protein of FTLD-U, is a neuronal activity-responsive factor. J. Neurochem., 105, 797-806.
22. Voigt, A., et al. (2010) TDP-43-mediated neuron loss in vivo requires RNA-binding activity. PLoS One, 5, e12247.
23. Li, Y., et al. (2010) A Drosophila model for TDP-43 proteinopathy. Proc. Natl Acad. Sci. USA, 107, 3169-3174.
24. Hanson, K. A., et al. (2010) Ubiquilin modifies TDP-43 toxicity in a Drosophila model of amyotrophic lateral sclerosis (ALS). J. Biol. Chem., 285, 11068-11072.
25. Feiguin, F., et al. (2009) Depletion ofTDP-43 affects Drosophila motoneurons terminal synapsis and locomotive behavior. FEBS Lett., 583, 1586-1592.
26. Lu, Y., et al. (2009) Frontotemporal dementia and amyotrophic lateral sclerosis-associated disease protein TDP-43 promotes dendritic branching. Mol. Brain, 2, 30.
27. Ash, P. E., et al. (2010) Neurotoxic effects of TDP-43 overexpression in C. elegans. Hum. Mol. Genet., 19, 3206-3218.
28. Wegorzewska, I., et al. (2009) TDP-43 mutant transgenic mice develop features of ALS and frontotemporal lobar degeneration. Proc. Natl Acad. Sci. USA, 106, 18809-18814.
29. Kabashi, E., et al. (2009) Gain and loss of function of ALS-related mutations of TARDBP (TDP-43) cause motor deficits in vivo. Hum. Mol. Genet., 19, 671-683.
30. Liachko, N. F., Guthrie, C. R. and Kraemer, B. C. (2010) Phosphorylation promotes neurotoxicity in a Caenorhabditis elegans model of TDP-43 proteinopathy. J. Neurosci., 30, 16208-16219.
31. Brand, A. H., et al. (1993) Targeted gene expression as a means of altering cell fates and generating dominant phenotypes. Development, 118, 401-415.
32. Arai, T., et al. (2006) TDP-43 is a component of ubiquitin-positive tau-negative inclusions in frontotemporal lobar degeneration and amyotrophic lateral sclerosis. Biochem. Biophys. Res. Commun., 351, 602-611.
33. Johnson, B. S., et al. (2009) TDP-43 is intrinsically aggregation-prone and ALS-linked mutations accelerate aggregation and increase toxicity. J. Biol. Chem., 284, 20329-20339.
34. Barmada, S. J., et al. (2010) Cytoplasmic mislocalization of TDP-43 is toxic to neurons and enhanced by a mutation associated with familial amyotrophic lateral sclerosis. J. Neurosci., 30, 639-649.
35. Gustafson, K., et al. (1996) Distinct expression patterns detected within individual tissues by the GAL4 enhancer trap technique. Genome, 39, 174-182.
36. Koh, Y. H., et al. (2000) Drosophila larval neuromuscular junction: molecular components and mechanisms underlying synaptic plasticity. Microsc. Res. Tech., 49, 14-25.
37. Ranjan, R., et al. (1998) Cysteine string protein is required for calcium secretion coupling of evoked neurotransmission in Drosophila but not for vesicle recycling. J. Neurosci., 18, 956-964.
38. Eaton, B. A., et al. (2005) LIM Kinase 1 controls synaptic stability downstream of the type II BMP receptor. Neuron, 47, 695-708.
39. Bodily, K. D., et al. (2001) A novel member of the Ig superfamily, turtle, is a CNS-specific protein required for coordinated motor control. J. Neurosci., 21, 3113-3125.
40. Le Bourg, E., et al. (1992) Hypergravity and aging in Drosophila melanogaster. 4. Climbing activity. Gerontology, 38, 59-64.
41. Neumann, M. (2009) Molecular neuropathology of TDP-43 proteinopathies. Int. J. Mol. Sci., 10, 232-246.
42. Belote, J. M., et al. (2002) Targeted expression of dominant negative proteasome mutants in Drosophila melanogaster. Genesis, 34, 80-82.
43. Warrick, J. M., et al. (1999) Suppression of polyglutamine-mediated neurodegeneration in Drosophila by the molecular chaperone HSP70. Nat. Genet., 23, 425-428.
44. Auluck, P. K., et al. (2002) Chaperone suppression of alpha-synuclein toxicity in a Drosophila model for Parkinson's disease. Science, 295, 865-868.
45. Jin, P., et al. (2003) RNA-mediated neurodegeneration caused by the fragile X premutation rCGG repeats in Drosophila. Neuron, 39, 739-747.
46. Zhou, L., et al. (1997) Cooperative functions of the reaper and head involution defective genes in the programmed cell death of Drosophila central nervous system midline cells. Proc. Natl Acad. Sci. USA, 94, 5131-5136.
47. Elden, A. C., et al. (2010) Ataxin-2 intermediate-length polyglutamine expansions are associated with increased risk for ALS. Nature, 466, 1069-1075.
48. Ritson, G. P., et al. (2010) TDP-43 mediates degeneration in a novel Drosophila model of disease caused by mutations in VCP/p97. J. Neurosci, 30, 7729-7739.
49. Yang, C., Tan, W., Whittle, C., Qiu, L., Cao, L., Akbarian, S. and Xu, Z. (2010) The C-terminal TDP-43 fragments have a high aggregation propensity and harm neurons by a dominant-negative mechanism. PLoS ONE, 5, e15878.
50. Jin, P., et al. (2004) Biochemical and genetic interaction between the fragile X mental retardation protein and the microRNA pathway. Nat. Neurosci., 7, 113-117.
51. Zarnescu, D. C., et al. (2005) Fragile X protein functions with lgl and the par complex in flies and mice. Dev. Cell, 8, 43-52.
52. Sephton, C. F., et al. (2011) Identification of neuronal RNA targets of TDP-43-containing ribonucleoprotein complexes. J. Biol. Chem., 286, 1204-1215.
53. Zhou, H., et al. (2010) Transgenic rat model of neurodegeneration caused by mutation in the TDP gene. PLoS Genet., 6, e1000887.
54. Cairns, N. J., et al. (2010) TDP-43 proteinopathy in familial motor neuron disease with TARDBP A315T mutation: a case report. Neuropathol. Appl. Neurobiol., 36, 673-679.
55. Estes, P. S., et al. (1996) Traffic of dynamin within individual Drosophila synaptic boutons relative to compartment-specific markers. J. Neurosci., 16, 5443-5456.

### 7. Example 4

Amyotrophic lateral sclerosis (ALS) is a fatal disease characterized by complex neuronal and glial phenotypes. Disclosed herein is a Drosophila model of ALS based on TDP-43 that recapitulates several aspects of pathology, including motor neuron loss, locomotor dysfunction and reduced survival. Also disclosed are the phenotypic consequences of expressing wild-type and four different ALS-linked TDP-43 mutations in neurons and glia. TDP-43-driven neurodegeneration phenotypes were dose-dependent and age-dependent. In motor neurons, TDP-43 appeared restricted to nuclei, which were significantly misshapen due to mutant but not wild-type protein expression. In glia and in the developing neuroepithelium, TDP-43 associated with cytoplasmic puncta. TDP-43-containing RNA granules were motile in cultured motor neurons, although wild-type and mutant variants exhibited different kinetic properties. Regarding neuromuscular junctions, the expression of TDP-43 in motor neurons versus glia lead to opposite synaptic phenotypes that translated into comparable locomotor defects. Finally, sleep was explored as a behavioral readout of TDP-43 expression and evidence of sleep fragmentation consistent with hyperexcitability was found. These data indicate that although motor neurons and glia are both involved in ALS pathology, at the cellular level they can exhibit different responses to TDP-43. These data indicate that individual TDP-43 alleles utilize distinct molecular mechanisms, which can influence strategies for developing therapeutics.

Amyotrophic lateral sclerosis (ALS) is a progressive neurodegenerative disorder that affects motor neurons and leads to paralysis and respiratory failure, followed by death usually within 2-5 years of diagnosis. Familial ALS (fALS) affects 10% of patients and the remaining 90% of ALS cases are sporadic (sALS) and remain poorly understood. To date, the most common gene linked to ALS is C90RF72, whose mechanism of action appears to be linked to RNA dysregulation (DeJesus-Hernandez et al., 2011; Renton et al., 2011). Several other loci have been linked to both fALS and sALS and include SOD1, alsin, senataxin, VAMP/synaptobrevin-associated protein B, P150 dynactin, angiogenin, TAR DNA-binding protein (TDP-43), Fused in Sarcoma (FUS) and profilin (Alexander et al., 2002; Beleza-Meireles and Al-Chalabi, 2009; Lagier-Tourenne and Cleveland, 2009; Wu et al., 2012).

Based on the known functions of these loci and extensive phenotypic studies in model organisms, ALS appears to be the result of abnormalities in diverse cellular processes ranging from oxidative stress, intracellular transport, RNA metabolism, apoptosis and actin dynamics. Of these, RNA-based mechanisms have recently taken center stage in the ALS field due to findings that the RNA-binding proteins such as TDP-43 and FUS constitute markers of pathology and, in addition, when mutated, neural degeneration occurs in human patients (Colombrita et al., 2011; Kabashi et al., 2008; Kwiatkowski et al., 2009; Sreedharan et al., 2008; Vance et al., 2009). Studies in animal models including worms, flies, zebrafish and rodents support the notion that alterations in these RNA-binding proteins and in RNA metabolism cause motor neuron disease (Couthouis et al., 2011; Estes et al., 2011; Lanson et al., 2011; Li et al., 2010; Liachko et al., 2010; Lu et al., 2009; Wegorzewska et al., 2009). Together with the recent discovery of GGGGCC repeat RNA caused by expansions in C9ORF72, these studies indicate that RNA dysregulation is an important aspect of ALS (DeJesus-Hernandez et al., 2011; Lagier-Tourenne and Cleveland, 2009; Renton et al., 2011).

Of the RNA-binding proteins linked to ALS, TDP-43 has first captured the attention of the field due to pathological studies that identified it as a component of cytoplasmic inclusions in neurons and the surrounding glia (Maekawa et al., 2009; Neumann et al., 2006; Tan et al., 2007). Subsequently, TDP-43 was shown to harbor several missense mutations linked to ALS, some of which are studied here using Drosophila as a model (Kabashi et al., 2008; Rutherford et al., 2008; Sreedharan et al., 2008). Structurally, TDP-43 consists of two RNA recognition motifs (RRM1 and RRM2) and a glycine-rich domain within the C terminus (Johnson et al., 2008). Its known cellular functions include transcriptional repression, splicing, miRNA biogenesis and apoptosis (Banks et al., 2008). In vitro assays and RNA sequencing approaches have shown that TDP-43 binds UG-rich sequences with high affinity and regulates splicing of numerous targets, including its own transcript (Ayala et al., 2011; Buratti and Baralle, 2001; Polymenidou et al., 2011; Sephton et al., 2011; Tollervey et al., 2011). TDP-43 is ubiquitously expressed and associates with other RNA-binding proteins (Freibaum et al., 2010). In cultured neurons, TDP-43 associates with neuronal RNA granules and colocalizes with RNA-binding proteins such as Fragile X protein (FMRP) and Staufen in an activity-dependent manner, which indicates that TDP-43 regulates the expression of synaptic mRNAs (Fallini et al., 2012; Wang et al., 2008; Yu et al., 2012).

The majority of TDP-43 mutations found in ALS patients represent amino acid substitutions that are thought to increase TDP-43 phosphorylation and target it for degradation (Kabashi et al., 2008; Rutherford et al., 2008; Sreedharan et al., 2008). These missense mutations can mimic a loss of function for TDP-43 and that the RNA-binding domain is required to mediate neurotoxicity (Estes et al., 2011; Feiguin et al., 2009; Voigt et al., 2010). Different TDP-43 mutations can differentially regulate stress granule formation in cell lines subjected to environmental stress (Dewey et al., 2011; Liu-Yesucevitz et al., 2010; McDonald et al., 2011).

A battery of phenotypic assays were used with wild-type and mutant A315T TDP-43, which exhibited differential levels of neurotoxicity in the Drosophila model of ALS disclosed herein. These data indicate that different TDP-43 alleles utilized distinct mechanisms in motor neuron disease. The studies disclosed herein compared the phenotypic consequences of expressing wild-type TDP-43 and four different variants (D169G, G298S, A315T and N345K) in motor neurons versus glia, in vivo, using the disclosed Drosophila model. The D169G mutation lies within the first RNA-binding domain of TDP-43 (RRM1) and has been linked to sporadic ALS, whereas G298S, A315T and N345K reside within the C-terminus domain of TDP-43 and have been linked to familial ALS (Neumann, 2009). Using the bipartite Gal4-UAS expression system in Drosophila, the expression of D169G, G298S and N345K mutations in the eye neuroepithelium led to a dose- and age-dependent neurodegeneration. The presence of TDP-43 puncta in photoreceptor axons was confirmed. TDP-43 was detectable in insoluble aggregates. The effects of TDP-43 expression in motor neurons versus glia were compared. These cells handled TDP-43 expression differently, both in a cell type- and a variant-dependent manner. In an aspect, TDP-43 was primarily restricted to nuclei in motor neurons in vivo, whereas in glial cells, TDP-43 puncta were detected in the cytosol including the glial cytoplasmic extensions (i.e., glial ‘feet’) that enveloped the neuromuscular junction synapse. The motor neuron nuclei were grossly misshapen whereas glial nuclei exhibited milder shape abnormalities. These data indicate that TDP-43 elicited different responses in glial cells than in motor neurons.

Upon culturing primary motor neurons from the ventral ganglia, cytoplasmic TDP-43-positive puncta were detected, consistent with the notion that these structures represent stress granules, which can have protective role (Dormann and Haass, 2011). These TDP-43 cytoplasmic puncta were motile and exhibited dynamic features typical of neuronal RNA transport granules. Fluorescence recovery after photobleaching (FRAP) experiments showed that wild-type TDP-43 exhibited different kinetic properties to the mutant TDP-43 alleles used. The consequences of expressing TDP-43 in motor neurons versus glia on the architecture as well as the synaptic marker distribution at the larval neuromuscular junction were compared. These experiments indicated profound phenotypic differences, including opposite effects on the number of active zones and glutamate receptor distribution. Larval locomotor activity, a measure of synaptic function, appeared to be affected by TDP-43 expression in both motor neurons and glia. Finally, adult activity and sleep patterns were examined and evidence of locomotor dysfunction and sleep fragmentation was found.

These experiments provide an in vivo comparison of several neuroanatomical, cellular, and functional phenotypes due to motor neuron versus glial expression of multiple TDP-43 variants linked to ALS in humans.

### a. Overexpression of Several Mutant Forms of TDP-43 LED to Age- and Dose-Dependent Neurodegeneration in the Eye

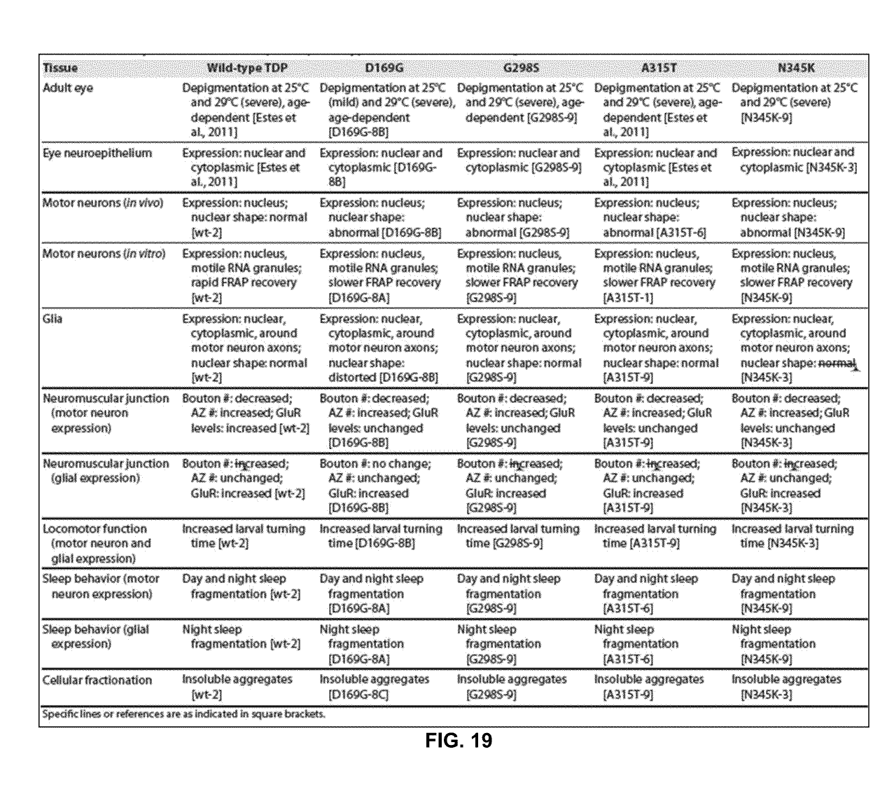

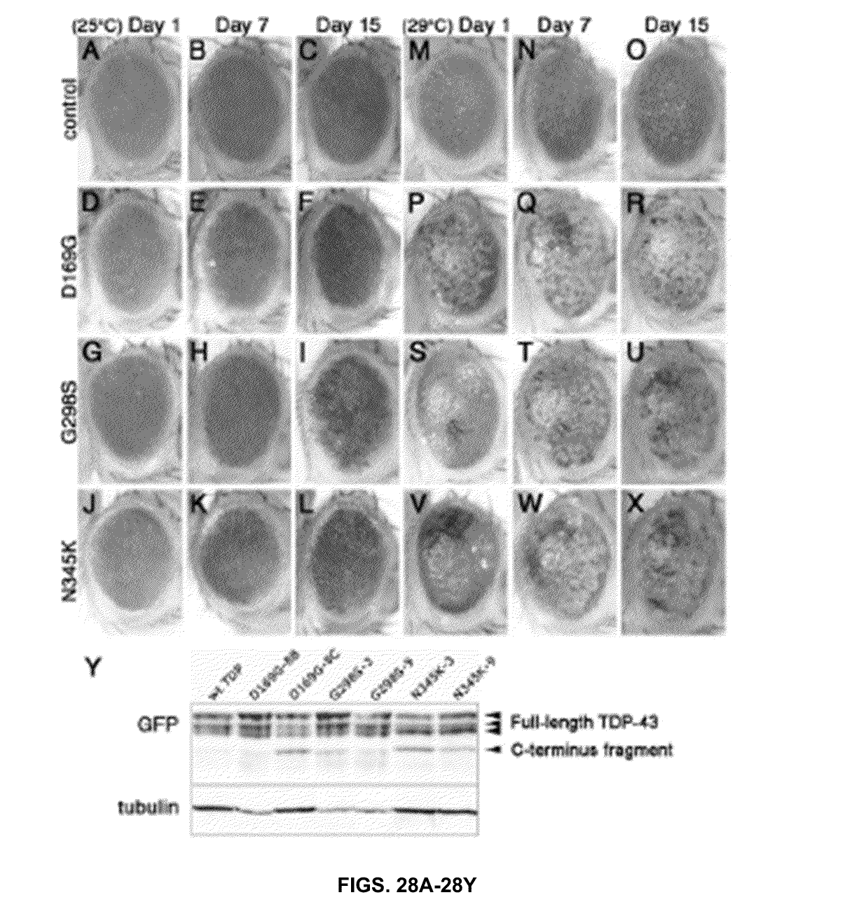

Overexpression of human wild-type TDP-43 or the ALS-linked variant A315T TDP-43 in the developing neuroepithelium resulted in neurodegeneration. The phenotypic consequences of expressing additional missense mutations linked to ALS, namely D169G, G298S and N345K in the retina were examined. With the exception of D169G, which lies within the first RNA-binding domain RRM1 and has been linked to sporadic ALS, the remainder of the mutations used in this study have been linked to familial ALS and are found within the C-terminus domain of TDP-43 (Kabashi et al., 2008). For each variant, several transgenic lines were surveyed and those that exhibited similar levels of expression were selected (for a representative set, see FIG. 28Y). Some mutant variant lines displayed clearly visible truncated C-terminal fragments (˜25 kDa) while other lines only showed a modest or barely detectable truncation (FIG. 28Y). Using the bipartite Gal4-UAS system, overexpression of all TDP-43 variants in the developing retina resulted in progressive neurodegeneration (as indicated by depigmentation, FIG. 28D-L) compared with GMR Gal4 controls (FIG. 28A-C). The D169G mutant exhibited a milder phenotype (especially at day 15, compare FIG. 28D-F with 28G-L). These phenotypic differences appeared less pronounced when TDP-43 levels were increased by raising the flies at a higher temperature (29° C. versus the standard 25° C.), which led to an enhanced neurodegeneration in the retina (compare FIG. 28A-L with 28M-X). This phenotypic enhancement can be explained by higher levels of TDP-43, which were due to an increase in Gal4 activity at higher temperatures. These results demonstrated that the D169G, G298S and N345K variants were toxic in vivo and were consistent with data indicating that TDP-43 expression in the retina led to an age- and dose-dependent neurodegeneration phenotype. These data further indicated that the D169G mutation in the RRM1 domain exhibited a lower level of toxicity than other variants, at least in the retina (see FIG. 19 for a summary of variant-specific phenotypes).

### b. TDP-43 Localized to Axonal Aggregates in the Developing Neuroepithelium

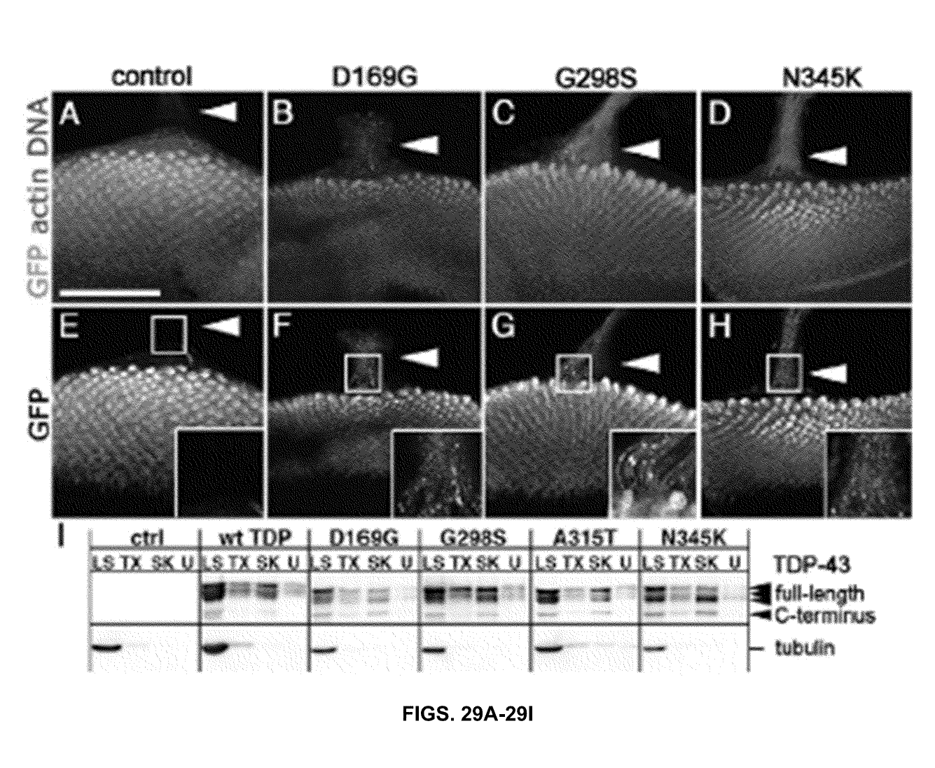

A hallmark of ALS pathology is the presence of cytoplasmic aggregates containing TDP-43 (Neumann et al., 2006). The distribution of TDP-43 in the eye neuroepithelium cytoplasm was evaluated. TDP-43-positive structures were visible in axons for all variants tested here (FIG. 29A-H, high magnification insets in E-H). To determine whether these corresponded to insoluble aggregates found in patient samples, a cellular fractionation approach was taken. All the TDP variants were largely found in low salt (LS) or detergent-soluble fractions (Triton X-100 and Sarkosyl fractions in FIG. 29I). A relatively small amount of TDP-43 was found in detergent-insoluble aggregates (urea fraction in FIG. 29I). Most of the phosphorylated mutant TDP-43 was found in the detergent-soluble (e.g., Triton X-100 and Sarkosyl) and insoluble (i.e., urea) fractions, which correlated with more misfolded protein (Liachko et al., 2010). These data indicated a positive correlation between photoreceptor neurodegeneration and the accumulation of TDP-43 in cytoplasmic puncta, some of which were insoluble aggregates.

### c. TDP-43 Overexpression in Motor Neurons In Vivo Did not Result in Detectable Cytoplasmic Puncta but Affected Nuclear Morphology

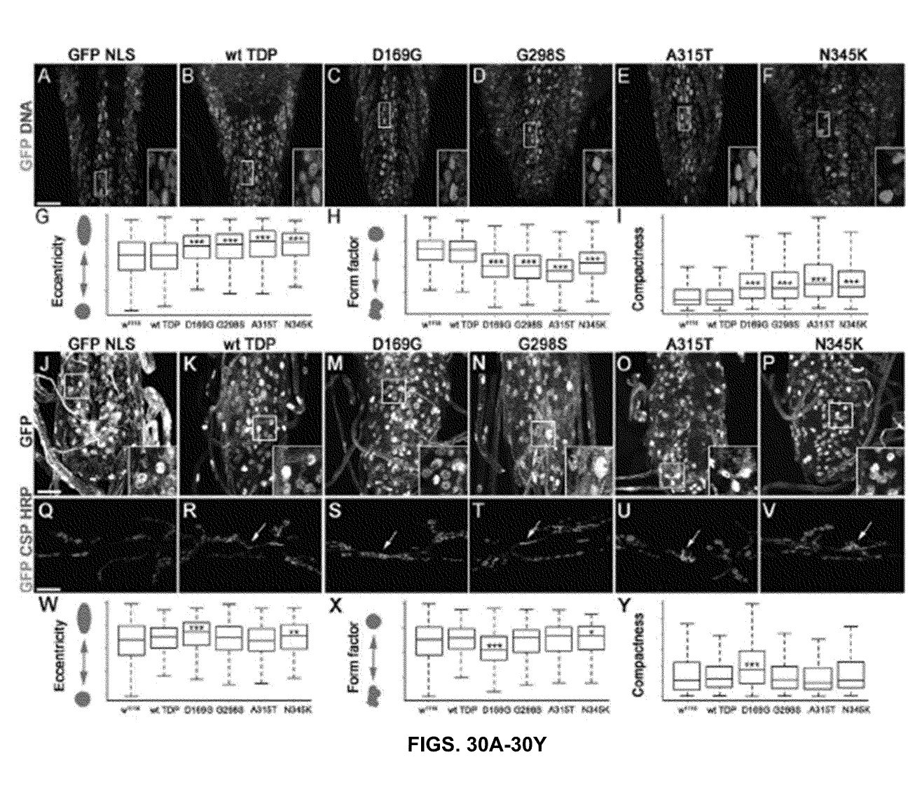

The localization of TDP-43 variants when expressed in motor neurons was examined using the D42 Gal4 driver (FIG. 30A-F). TDP-43 appeared mostly restricted to the nucleus compared with controls (compare FIG. 30B-F with 30A). Upon close examination, motor neuron nuclei expressing TDP-43 were misshapen, a feature associated with changes in gene expression and apoptosis (FIG. 30A-F, insets). Using Cell Profiler software, eccentricity, form factor, and compactness were quantified (all of which measured shape), of nuclei expressing TDP-43 in comparison to control nuclei (which appeared round and smooth). As shown in FIG. 30G-I, all mutant TDP variants used in this study (D169G, G298S, A315T and N345K) exhibited a significantly higher eccentricity and compactness compared with D42 driver controls. TDP-43 mutations led to a reduction in form factor, which was consistent with nuclear malformation from a sphere to a random three-dimensional shape (FIG. 30H). These results indicated that the overexpression of TDP-43 mutations in motor neurons results in shape abnormalities and distorted nuclei. All mutant variants exhibited stronger phenotypes than wild-type TDP-43, indicating that this assay can be useful in distinguishing variant-specific effects of TDP-43 in motor neurons in vivo.

### d. TDP-43 Formed Cytoplasmic Puncta in Glia but Had Little or No Effect on Nuclear Shape

Recent evidence indicates that glial cell toxicity has a contribution to ALS pathology (Ince et al., 2011). To determine whether TDP-43-mediated pathology has a glial component, wild-type and mutant TDP-43 were expressed in glial cells using the panglial driver repo Gal4 (Xiong et al., 1994). In contrast to motor neurons, it was found that TDP-43 exits the nucleus and localizes to cytoplasmic puncta in glial cells (FIG. 30J-P) that can be observed as far from the nucleus as the glial ‘feet’ surrounding the neuromuscular junction synapse (Fuentes-Medel et al., 2009) (FIG. 30Q-V, arrows). Upon examining the glial nuclei, only D169G and N345K TDP had a significant effect on nuclear shape (see insets in FIG. 30J-P and see 30W-Y for quantification). These data indicated an inverse correlation between nuclear restriction or cytoplasmic localization and nuclear shape distortion due to TDP-43 expression in glia and provide insights into phenotypic differences between TDP-43 variants.

### e. TDP-43 Formed Dynamic Puncta in Motor Neurons in Culture

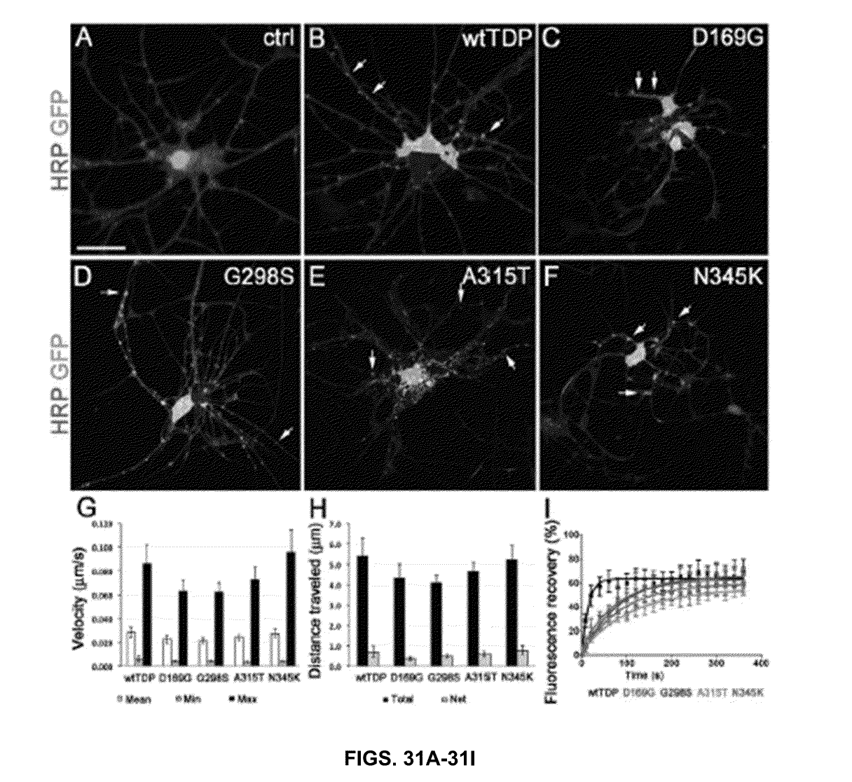

Next, the issue of the subcellular localization of TDP-43 was addressed using a primary culture approach. TDP-43 in cytoplasmic puncta in motor neurons were not detected in vivo. TDP-43 formed well-defined puncta distributed throughout the soma and neurites (FIG. 31B-F, arrows). D169G-containing puncta appeared to be less defined (compare FIG. 31C with 31B,D-F).

Using live imaging in primary motor neurons, the dynamic properties of several TDP-43 variants were compared and it was found that TDP-43 puncta undergo transport at rates comparable to neuronal RNA granules containing Fragile X protein (FMRP) (Estes et al., 2008) (FIG. 31G). In this assay, there were not any significant differences detected between wild-type and mutant TDP-43, with granule speeds that ranged between 0.01 and 0.1 μm/second, consistent with microtubule-based transport. Similarly, total and net distances traveled within neurites were comparable among the TDP-43 variants used in this study (4.1-5.4 μm for total and 0.4-0.8 μm for net distances; FIG. 31H). To further characterize the kinetics of TDP-43 in neurites, FRAP was used. FRAP determines the ability of TDP-43 to shuttle in and out of stationary RNA granules (i.e., mobility). These experiments indicated that TDP-43 was highly mobile within neurites, as indicated by a 55-64% recovery of the initial fluorescence intensity (FIG. 31I). Wild-type TDP-43 exhibited significantly different dynamics compared to mutant TDP-43, with a recovery half-time of 9.7 seconds compared with 51.2 seconds for D169G, 56.8 seconds for G298S, 76.1 seconds for A315T and 62.0 for N345K TDP-43. These results were consistent with the experimental observations that wild-type TDP-43 granules are difficult to bleach down to 20% or less fluorescence (which was the set threshold for bleaching RNA granules with the laser) due to their fast recovery.

### f. TDP-43 Overexpression in Motor Neurons or Glia Affected Axonal Growth and Locomotor Function at the Neuromuscular Junction

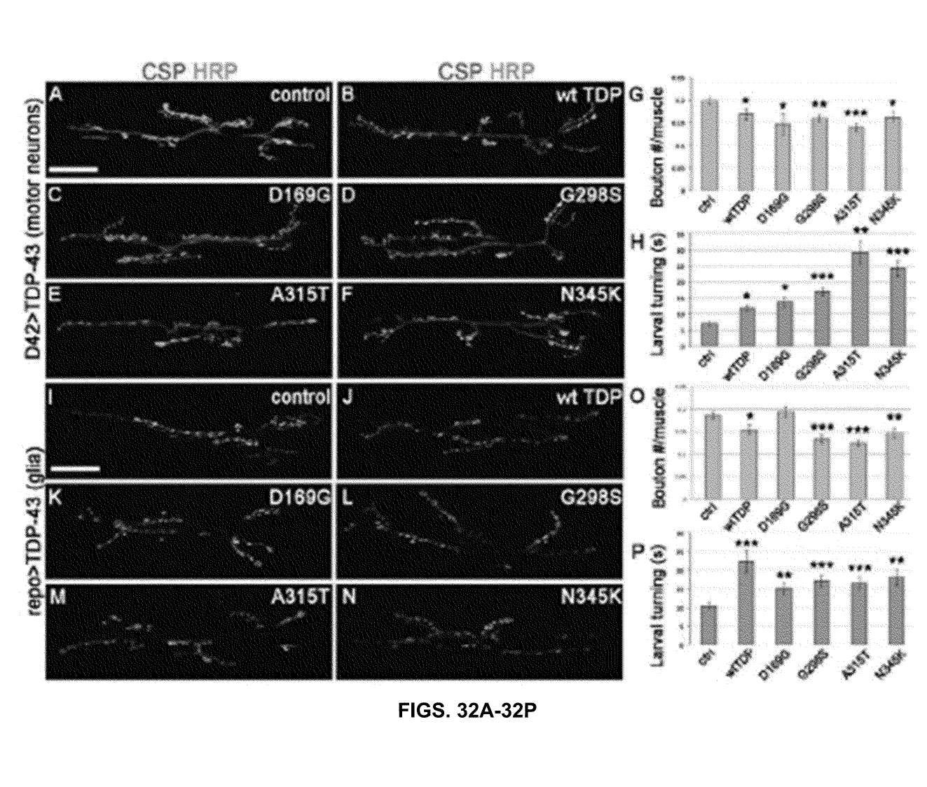

TDP-43 is required for the architecture of the larval neuromuscular junction synapse (NMJ). Each NMJ contains several varicosities referred to as synaptic boutons, which form as motor neuron axons innervate the larval body wall musculature. The synaptic vesicle marker cysteine string protein (CSP) (Bronk et al., 2005) and the neuronal membrane marker horseradish peroxidase (HRP) were examined. The examination focused on the well-characterized synapses at muscles 6/7 in abdominal segment 3 (Koh et al., 2000). The effects of overexpressing wild-type as well as D169G, G298S, A315T and N345K TDP-43 in motor neurons (FIG. 32A-F) or glia (FIG. 32I-N) on the morphology of the NMJ were compared. When expressed in motor neurons, all TDP-43 variants tested resulted in smaller synapses, as indicated by a reduced number of synaptic boutons (FIG. 32G). This anatomical phenotype was accompanied by impairment in locomotor function, as indicated by a significant increase in larval turning time compared with controls (FIG. 32H).

Similarly, when expressed in glial cells, all variants except D169G TDP-43 resulted in significantly smaller NMJs, with a decreased number of synaptic boutons (FIG. 320). When tested for their ability to turn, larvae expressing TDP-43 in glia also exhibited a significant impairment in locomotor function (FIG. 32P). These data show that, when expressed in either motor neurons or glia, the phenotypic consequences of TDP-43 on synaptic architecture and locomotor function were comparable.

### g. Synaptic Markers were Differentially Affected by TDP-43 Expression in Motor Neurons Versus Glia

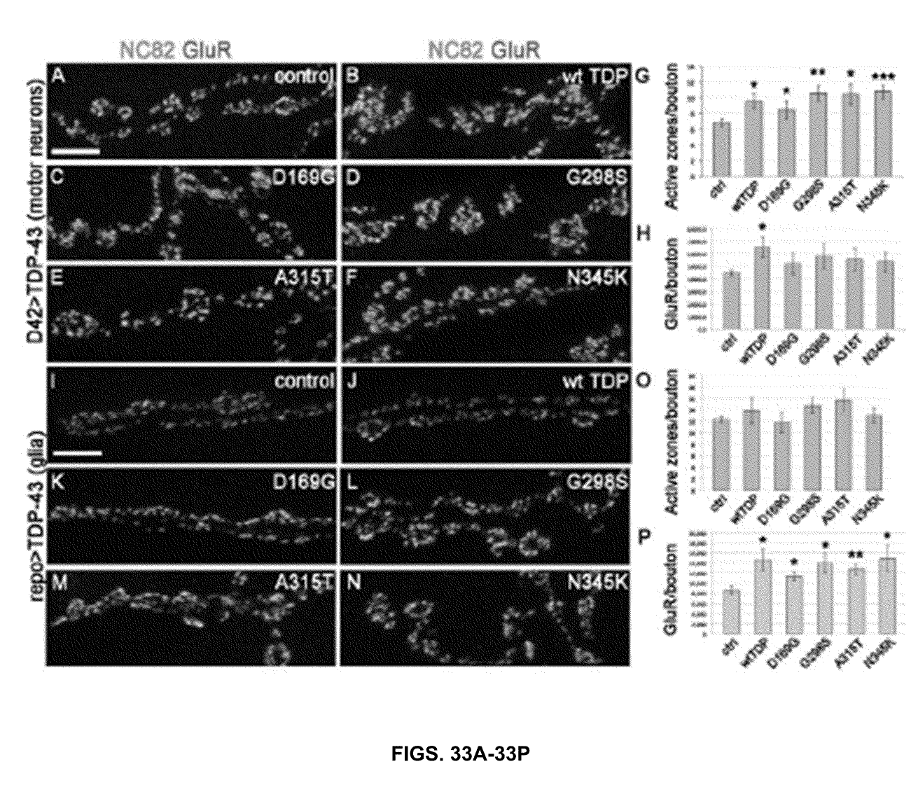

The findings that TDP-43 modulated the architecture of the NMJ indicate potential effects on synaptic function. Thus, the distribution of various synaptic markers including Bruchpilot (a core component of active zones recognized by the NC82 antibody) (Kittel et al., 2006; Wagh et al., 2006) and the glutamate receptor GluRIIC (Marrus and DiAntonio, 2004) were examined. In wild-type larvae, there was generally a 1:1 correspondence of pre-synaptic active zones to post-synaptic glutamate receptor densities, which contributes to the efficient relay of synaptic signals from motor neurons to muscles. As shown in FIG. 33, TDP-43 overexpression in motor neurons led to a significant increase in active zones (areas of neurotransmitter release) compared with controls (see NC82 staining in FIG. 33A-F and quantification in 33G). In contrast, there was no significant effect on glutamate receptor levels (as measured by quantitative confocal fluorescence) when TDP-43 mutations were expressed in motor neurons (FIG. 33H). An increase in glutamate receptor levels was found due to wild-type TDP-43 expression, which provides further support for the notion that the TDP-43 mutants utilize distinct cellular mechanisms. These data indicate an impairment in synaptic function at the NMJ due to overexpression of TDP-43 in motor neurons and explain the larval turning defect shown in FIG. 32H.

Next, the synaptic phenotypes due to TDP-43 overexpression in glia were examined. There were no obvious changes in the number and distribution of pre-synaptic active zones (FIG. 33I-N, O), but the levels of post-synaptic glutamate receptor were significantly increased (FIG. 33I-N, P). These data indicated a non-autonomous role for TDP-43 in glia and in modulating synaptic properties. There data were consistent with the locomotor function defects observed when TDP-43 was overexpressed in glia (FIG. 32P). These findings showed that when expressed in either motor neurons or glia, TDP-43 leads to a mismatch, albeit in opposite direction between pre-synaptic active zones and post-synaptic glutamate receptor densities, which in turn, translated into synaptic and locomotor dysfunction.

### h. Sleep and Locomotor Activity in Adult Flies were Impaired Due to TDP-43 Expression

In addition to impaired motor function, ALS patients exhibit sleep disturbances (Lo Coco et al., 2011). To determine whether sleep patterns are also affected in the Drosophila model disclosed herein, Drosophila Activity Monitors (DAMs) were used. Both locomotor and sleep activity in adult flies expressing TDP-43 were quantified. DAMs are kept at 25° C. in an incubator equipped with 12-hour alternating dark-light cycles and can hold several long and narrow glass tubes filled with food on one end, plugged with cotton at the other end and housing one fly each.

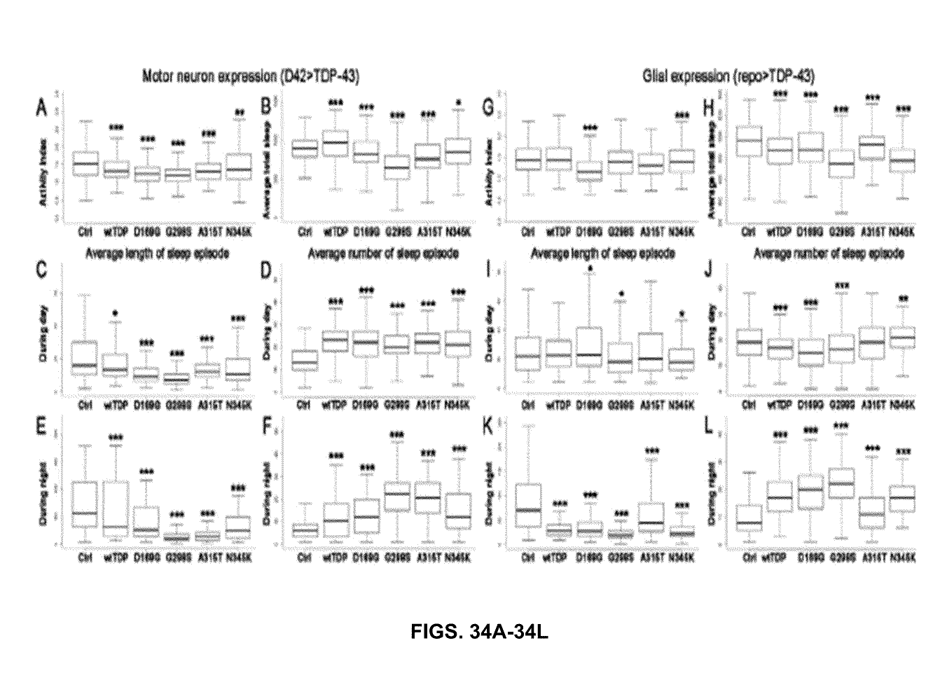

As a fly walked back and forth within its tube, it interrupted an infrared beam that crossed the tube at its midpoint and this interruption, detected by the onboard electronics, was added to the tube's activity count as a measure of fly activity. This daily record was simultaneously acquired from several flies and provided a good measure of both the intensity of locomotor activity and the relative periods of rest. A period of rest of 5 minutes or more was defined as sleep (Sehgal and Mignot, 2011). Locomotor activity was evaluated for populations of flies with the same genotype as an average activity over time using Pysolo, a multi-platform software for analyzing sleep and locomotor patterns in Drosophila (Gilestro and Cirelli, 2009). As seen in FIG. 34, these experiments showed that TDP-43 expression in motor neurons led to a significant reduction in locomotor activity (FIG. 34A). In addition, total sleep was altered, with wild-type TDP-43-expressing flies sleeping more that those expressing mutant TDP-43, which exhibited a reduction in total sleep (FIG. 34B). When the sleep data was analyzed, adults expressing TDP-43 variants exhibited, on average, shorter sleep episodes both during day and night (FIG. 34C,E), but exhibited significantly more sleep episodes (FIG. 34D,F).

When expressed in glia, TDP-43 also led to alterations in locomotor activity, although only D169G and N345K were significantly different from controls (FIG. 34G). With regard to sleep activity, total sleep was significantly reduced for all variants used in this study (FIG. 34H). During the day, alterations in both the number of sleep episodes and their duration were found; however significant differences were not consistently detected (FIG. 34I, J). In contrast, during the night, significantly more (FIG. 34L), albeit shorter, sleep episodes (FIG. 34K) were found for all TDP-43-expressing flies compared with controls. These results indicated that both motor neuron and glial expression of TDP-43 led to alterations in adult locomotion and sleep patterns, although glial expression was mildly less toxic, especially during the day. These data were consistent with sleep fragmentation and parallel the disturbances in sleep patterns reported in patients (Lo Coco et al., 2011)

### i. Summary of Results for Example 4

In human patients, ALS is primarily diagnosed by eliminating other known motor neuron or neurodegenerative conditions. This is due in part to a lack of biomarkers and in part due to the presence of complex and variable phenotypes, an issue further compounded by genetic background effects in sporadic ALS, which represents the majority of cases. Disclosed herein is a Drosophila model of ALS based on TDP-43, an RNA-binding protein that has emerged as a common denominator for most ALS cases known to date, due to its presence in cytoplasmic inclusions as well as the discovery of point mutations in ALS patients (Baloh, 2011; Colombrita et al., 2011; Fiesel and Kahle, 2011; Neumann, 2009).

The overexpression of the D169G, G298S and N345K variants in the developing neuroepithelium led to a progressive and dose-dependent neurodegeneration. The D169G mutation, which lied in the RRM1 domain, appeared less toxic in the eye than the other alleles, consistent with the notion that RNA-binding mediated by the RRM1 domain is important for mediating neurotoxicity (Voigt et al., 2010). D169G behaved differently to the other TDP-43 variants in multiple phenotypic assays. For example, there appeared to be less defined D169G puncta in primary motor neurons compared with other variants, indicating that the association with neuronal RNA granules was dependent on a fully functional RRM1 domain. Conversely, when TDP-43 variants were expressed in glia, the D169G mutant was one of two (the other was N345K) that showed significant effects on nuclear shape and adult locomotor activity (see FIG. 20 for a summary of phenotypes). Furthermore, D169G expression in glia had no effect on synaptic size, as indicated by the number of synaptic boutons that were similar to controls.

In other assays, similar phenotypic effects were generated by the different TDP-43 mutations, including the presence of cytoplasmic puncta in photoreceptor axons, a detectable truncated C-terminal fragment, nuclear shape defects and transport kinetics when expressed in motor neurons. Larval locomotor function and the distribution of synaptic markers such as presynaptic active zones and postsynaptic glutamate receptors were also affected by the different TDP-43 variants used in this study. When expressed in motor neurons, wild-type TDP-43 showed a different effect to that of the mutant variants on nuclear shape, transport kinetics, and glutamate receptor distribution. These findings support the assertion that various TDP-43 alleles are divergent and underscore the importance of determining their in vivo phenotypes.

The direct comparison of the effects of TDP-43 in motor neurons and glia showed that these different cell types exhibited a differential response to TDP-43 expression. TDP-43 appeared restricted to the nucleus in motor neurons but formed cytoplasmic puncta in glial cells in vivo. These differences in subcellular localization translated into differential effects on nuclear shape, which was often an indicator of cellular health (Dahl et al., 2008). Furthermore, motor neurons and glia appeared to handle TDP-43 expression by differentially modulating the distribution of synaptic proteins including Bruchpilot, a presynaptic active zone marker CSP and postsynaptic glutamate receptors. The significant increase in active zones due to TDP-43 overexpression in motor neurons was not matched by a similar increase in glutamate receptors, indicating that excess glutamate was released in the synaptic cleft. This was consistent with increased excitability. When TDP-43 was expressed in glia, there was an increase in postsynaptic glutamate receptor levels but not in presynaptic active zones. This mismatch between the pre- and postsynaptic sides of the neuromuscular junction was consistent with the functional defects identified using larval turning assays. These findings demonstrated that TDP-43 expression in glia was also toxic with respect to locomotor function.

The side-by-side comparison of motor neuron and glial expression showed that adult locomotor activity was also impaired in both conditions. TDP-43 toxicity in glia appeared to be slightly milder due (in part) to differences in levels of expression between the D42 and repo Gal4 lines that were used to drive expression in motor neurons and glia, respectively. Total sleep time was similarly affected by TDP-43 expression in both motor neurons and glia, indicating that the sleep phenotypes were less sensitive to differences between Gal4 driver levels of expression. These studies uncovered a sleep fragmentation phenotype similar to that reported in ALS patients.

In summary, the data generated herein using the disclosed Drosophila model of ALS showed that both motor neurons and glia were susceptible to TDP-43 toxicity. Moreover, distinct cell types appeared to mount different responses to TDP-43 expression. TDP-43 was required autonomously in motor neurons and non-autonomously in glia to regulate various morphological and functional aspects of the neuromuscular junction.

### 8. Experimental Design for Example 4

### A. Drosophila Genetics

All Drosophila stocks and crosses were kept on standard yeast/cornmeal/molasses food at 25° C. unless otherwise noted. Human TDP-43, wild-type and D169G, G298S, A315T, and N345K mutants with YFP C-terminal tags (originally in pRS416 yeast expression vector, from Aaron Gitler, Stanford University, CA) were cloned into the pUAST germline transformation vector and transgenic lines were made as disclosed herein. Gal4 drivers used in this study included the eye-specific GMR Gal4 R13, the motor neuron driver D42 Gal4 (Gustafson and Boulianne, 1996) and the glial-specific driver repo (Xiong et al., 1994). Several insertions were surveyed for each TDP-43 variant to ensure that the phenotypes observed in the eye are caused by TDP-43 and not due to positional effects. One or two lines for each variant were chosen for further study in individual assays (see FIG. 20). For controls, w1118 or UAS GFP NLS was crossed with the appropriate Gal4 drivers. The expression of GFP NLS resulted in mild phenotypes, presumably due to the accumulation of high levels of GFP in the nucleus compared with TDP-43, which was expressed at lower levels than GFP NLS and also shuttles in and out of the nucleus. Furthermore, because untagged and tagged wild-type TDP-43 produced comparable eye neurodegeneration and larval turning defects, for quantification purposes Gal4 driver crossed to w1118 were used as controls. GFP NLS was used to mark the cells where individual Gal4 lines are driving the expression of TDP-43.

### B. Western Blots

Western blots were performed as described herein. TDP-43 transgenes were detected using rabbit polyclonal anti-GFP (Invitrogen) at 1:3000. Tubulin was used as a loading control and was detected using a mouse monoclonal anti-tubulin antibody (Millipore) at 1:1000. Secondary antibodies were HRP-conjugated goat anti-mouse or goat anti-rabbit, as appropriate, at 1:1000 (Thermo Scientific).

### c. Immunohistochemistry and Imaging

Adult fly eyes were imaged with a Leica MZ6 microscope equipped with an Olympus DP71 camera and controlled by Olympus DP Controller and Olympus DP Manager software. Individual images were processed using Adobe Photoshop CS2 (Adobe). Larval neuromuscular junction preparations have been disclosed herein. Briefly, wandering third instar larvae were filleted in HL-3 saline, pinned out on Sylgard dishes and fixed in 3.5% formaldehyde in PBS, pH 7.2 for 20 minutes, then permeabilized with 0.1% Triton X-100. The blocking agent consisted of 2% BSA and 5% normal goat serum. The following antibodies were used: anti-DCSP2 at 1:300 (DSHB), anti-HRP-FITC at 1:50 (Sigma), anti Dlg polyclonal at 1:900 (gift from Peter Bryant, University of California, CA), anti-GluRC polyclonal at 1:2000 (gift from Aaron DiAntonio, Washington University, MO) and anti-Bruchpilot, NC82 at 1/50 (DSHB). Secondary antibodies were used at 1:1000 (Molecular Probes). Larval muscles 6-7 were imaged in abdominal segment A3 on a Nikon PCM 2000 or Zeiss Meta 510 confocal microscope and were displayed as projections of 1-μm serial sections. Boutons were manually counted and total CSP, glutamate receptor and HRP total pixel intensities were determined using Metamorph Image Analysis software (Universal Imaging). NC82 spot counts were performed using the Cell Counting Macro in the Metamorph software. All measurements were divided by total muscle area, and in some cases bouton number, to take into account variations in size of the individual larvae or synapse size.

Eye discs and larval ventral ganglia were dissected and prepared for immunohistochemistry as described above. Eye discs were stained with anti-GFP-FITC (Rockland) at 1:200, rhodamine phalloidin at 1:300 (Molecular Probes), as well as Hoechst 33342 (Invitrogen) at 1:10,000. Ventral ganglia were stained as above except that instead of phalloidin, anti-HRP-TRITC (Sigma) was used at 1:50. Samples were imaged on a Zeiss Meta 510 confocal microscope and displayed as projections of 1-μm serial sections.

Nuclear shape features were measured by CellProfiler 2.0, subversion 11710 (Carpenter et al., 2006) (pipeline provided upon request). Shape measurements of nuclei were quantified in CellProfiler as follows: nuclei stained with Hoechst 33342 were identified using the Otsu global threshold algorithm and filtered to represent those along the midline of the ventral ganglia. Shapes were then measured by their individual descriptions. Compactness is measured as the variance of the radial distance of the nuclei's perimeter from the centroid divided by the area. Form factor is measured as 4π×area/perimeter². Eccentricity is measured as the ratio of the distance between the foci of a fitted ellipse and its major axis length. Measurements were first checked for normality using the Anderson-Darling test and then for significant differences using t-tests for normal data and Wilcox tests for non-normal data. Data processing, statistical tests and box plots were computed by the statistical software package R, version 2.15.0 (R Core Development Team).

### d. Behavioral Assays

### (1) Larval Turning Assays

Assays were performed as disclosed herein. Briefly, wandering third instar larvae were placed on a grape juice plate at room temperature. After becoming acclimated, crawling larvae were gently turned onto their backs (ventral side up) and monitored until they were able to turn back (dorsal side up) and continue their forward movement. The amount of time that it took each larva to complete this task was recorded. Twenty to thirty larvae were used per genotype. Student's t-test was performed to assess statistical significance.

### (2) Sleep Assays

Adult flies of 1-3 days old were monitored individually in autoclavable plastic tubes (5 mm in diameter and 95 mm in length) on the same fly food described above under 12-hour light-dark cycles at 22° C. with 45-60% humidity. Preparation of individual fly vials was performed prior to the experiment by melting pre-made fly food to liquidity and ¾ filling plastic vial end-caps with the mixture. Vials were then inserted into the caps and the food was allowed to set for ˜30 minutes prior to use. Sleep and locomotor activity data were collected using the Drosophila Activity Monitoring (DAM) system (Trikinetics, Waltham, Mass.) and analyzed using Pysolo, a multiplatform software for analyzing sleep and locomotor activity in Drosophila (Gilestro and Cirelli, 2009). Flies were given 32 hours to habituate to the experimental conditions prior to data collection. Approximately 32 flies were gathered and placed in each monitor (maximum capacity 32) for each genotype for each experimental replicate. Sleep and locomotor activity data were collected at 1-minute intervals for 5 days. Three experimental replicates were performed and data from each replicate was compiled. Pre-processing of the Pysolo data was performed in Microsoft Excel. Normality of the data was tested using the Anderson-Darling test using statistical software package R. Based on the fact that at least one group per parameter was not normally distributed compared with the control, a non-parametric Wilcox test was used in R (R Core Development Team) to calculate all P values.

### e. Primary Neuron Cultures and Trafficking Measurements

Larval motor neurons were cultured as described (Estes et al., 2008) with the following exceptions: cells were isolated from wandering third instar larval ventral ganglia and imaged 5-6 days after plating at 25° C. Measurements of FRAP and speed were done as disclosed herein.

### f. Cellular Fractionations

Cellular fractionation was carried out as described in (Liachko et al., 2010) using as a starting material adult heads snap-frozen in liquid nitrogen and ground to a fine powder.

### g. References for Example 4

Alexander, M. D., et al. (2002). “True” sporadic ALS associated with a novel SOD-1 mutation. Ann. Neurol. 52, 680-683.
Ayala, Y. M., et al. (2008). TDP-43 regulates retinoblastoma protein phosphorylation through the repression of cyclin-dependent kinase 6 expression. Proc. Natl. Acad. Sci. USA 105, 3785-3789.
Ayala, Y. M., et al. (2011). TDP-43 regulates its mRNA levels through a negative feedback loop. EMBO J. 30, 277-288.
Baloh, R. H. (2011). TDP-43: the relationship between protein aggregation and neurodegeneration in amyotrophic lateral sclerosis and frontotemporal lobar degeneration. FEBS J. 278, 3539-3549.
Banks, G. T., et al. (2008). TDP-43 is a culprit in human neurodegeneration, and not just an innocent bystander. Mamm. Genome 19, 299-305.
Beleza-Meireles, A., et al. (2009). Genetic studies of amyotrophic lateral sclerosis: controversies and perspectives. Amyotroph. Lateral Scler. 10, 1-14.
Bronk, P., et al. (2005). The multiple functions of cysteine-string protein analyzed at Drosophila nerve terminals. J. Neurosci. 25, 2204-2214.
Buratti, E., et al. (2001). Characterization and functional implications of the RNA binding properties of nuclear factor TDP-43, a novel splicing regulator of CFTR exon 9. J. Biol. Chem. 276, 36337-36343.
Carpenter, A. E., et al. et al. (2006). CellProfiler: image analysis software for identifying and quantifying cell phenotypes. Genome Biol. 7, R100.
Colombrita, C., et al. (2011). RNA-binding proteins and RNA metabolism: a new scenario in the pathogenesis of Amyotrophic lateral sclerosis. Arch. Ital. Biol. 149, 83-99.
Couthouis, J., et al. (2011). A yeast functional screen predicts new candidate ALS disease genes. Proc. Natl. Acad. Sci. USA 108, 20881-20890.
Dahl, K. N., et al. (2008). Nuclear shape, mechanics, and mechanotransduction. Circ. Res. 102, 1307-1318.
DeJesus-Hernandez, M., al. (2011). Expanded GGGGCC hexanucleotide repeat in noncoding region of C9ORF72 causes chromosome 9p-linked FTD and ALS. Neuron 72, 245-256.
Dewey, C. M., et al. (2011). TDP-43 are directed to stress granules by sorbitol, a novel physiological osmotic and oxidative stressor. Mol. Cell. Biol. 31, 1098-1108.
Dormann, D., et al. (2011). TDP-43 and FUS: a nuclear affair. Trends Neurosci. 34, 339-348.
Estes, P. S., et al. (2008). Fragile X protein controls the efficacy of mRNA transport in Drosophila neurons. Mol. Cell. Neurosci. 39, 170-179.
Fallini, C., et al. (2012). The ALS disease protein TDP-43 is actively transported in motor neuron axons and regulates axon outgrowth. Hum. Mol. Genet. 21, 3703-3718.
Feiguin, F., et al. (2009). Depletion of TDP-43 affects Drosophila motoneurons terminal synapsis and locomotive behavior. FEBS Lett. 583, 1586-1592.
Fiesel, F. C., et al. (2011). TDP-43 and FUS/TLS: cellular functions and implications for neurodegeneration. FEBS J. 278, 3550-3568.
Freibaum, B. D., et al. (2010). Global analysis of TDP-43 interacting proteins reveals strong association with RNA splicing and translation machinery. J. Proteome Res. 9, 1104-1120.
Fuentes-Medel, Y., et al. (2009). Glia and muscle sculpt neuromuscular arbors by engulfing destabilized synaptic boutons and shed presynaptic debris. PLoS Biol. 7, e1000184.
Gilestro, G. F., et al. (2009). pySolo: a complete suite for sleep analysis in Drosophila. Bioinformatics 25, 1466-1467.
Gustafson, K., et al. (1996). Distinct expression patterns detected within individual tissues by the GAL4 enhancer trap technique. Genome 39, 174-182.
Ince, P. G., et al. (2011). Molecular pathology and genetic advances in amyotrophic lateral sclerosis: an emerging molecular pathway and the significance of glial pathology. Acta Neuropathol. 122, 657-671.
Johnson, B. S., et al. (2008). A yeast TDP-43 proteinopathy model: Exploring the molecular determinants of TDP-43 aggregation and cellular toxicity. Proc. Natl. Acad. Sci. USA 105, 6439-6444.
Kabashi, E., et al. (2008). TARDBP mutations in individuals with sporadic and familial amyotrophic lateral sclerosis. Nat. Genet. 40, 572-574.
Kittel, R. J., et al. (2006). Bruchpilot promotes active zone assembly, Ca2+ channel clustering, and vesicle release. Science 312, 1051-1054.
Koh, Y. H., et al. (2000). Drosophila larval neuromuscular junction: molecular components and mechanisms underlying synaptic plasticity. Microsc. Res. Tech. 49, 14-25.
Kwiatkowski, T. J., Jr., et al. (2009). Mutations in the FUS/TLS gene on chromosome 16 cause familial amyotrophic lateral sclerosis. Science 323, 1205-1208.
Lagier-Tourenne, C., et al. (2009). Rethinking ALS: the FUS about TDP-43. Cell 136, 1001-1004.
Lanson, N. A., Jr, et al. (2011). A Drosophila model of FUS-related neurodegeneration reveals genetic interaction between FUS and TDP-43. Hum. Mol. Genet. 20, 2510-2523.
Li, Y., et al. (2010). A Drosophila model for TDP-43 proteinopathy. Proc. Natl. Acad. Sci. USA 107, 3169-3174.
Liachko, N. F., et al. (2010). Phosphorylation promotes neurotoxicity in a Caenorhabditis elegans model of TDP-43 proteinopathy. J. Neurosci. 30, 16208-16219.
Liu-Yesucevitz, L., al. (2010). Tar DNA binding protein-43 (TDP-43) associates with stress granules: analysis of cultured cells and pathological brain tissue. PLoS ONE 5, e13250.
Lo Coco, D., et al. (2011). Sleep-wake disturbances in patients with amyotrophic lateral sclerosis. J. Neurol. Neurosurg. Psychiatry 82, 839-842.
Lu, Y., et al. (2009). Frontotemporal dementia and amyotrophic lateral sclerosis-associated disease protein TDP-43 promotes dendritic branching. Mol. Brain 2, 30.
Maekawa, S., et al. (2009). TDP-43 is consistently co-localized with ubiquitinated inclusions in sporadic and Guam amyotrophic lateral sclerosis but not in familial amyotrophic lateral sclerosis with and without SOD1 mutations. Neuropathology 29, 672-683.
Manus, S. B., et al. (2004). Preferential localization of glutamate receptors opposite sites of high presynaptic release. Curr. Biol. 14, 924-931.
McDonald, K. K., et al. (2011). TAR DNA-binding protein 43 (TDP-43) regulates stress granule dynamics via differential regulation of G3BP and TIA-1. Hum. Mol. Genet. 20, 1400-1410.
Neumann, M. (2009). Molecular neuropathology of TDP-43 proteinopathies. Int. J. Mol. Sci. 10, 232-246.
Neumann, M., et al. (2006). Ubiquitinated TDP-43 in frontotemporal lobar degeneration and amyotrophic lateral sclerosis. Science 314, 130-133.
Parker, S. J., et al. (2012). Endogenous TDP-43 localized to stress granules can subsequently form protein aggregates. Neurochem. Int. 60, 415-424.
Polymenidou, M., et al. (2011). Long pre-mRNA depletion and RNA missplicing contribute to neuronal vulnerability from loss of TDP-43. Nat. Neurosci. 14, 459-468.
Renton, A. E., et al. (2011). A hexanucleotide repeat expansion in C9ORF72 is the cause of chromosome 9p21-linked ALS-FTD. Neuron 72, 257-268.
Rutherford, N. J., et al. (2008). Novel mutations in TARDBP (TDP-43) in patients with familial amyotrophic lateral sclerosis. PLoS Genet. 4, e1000193.
Sehgal, A., et al. (2011). Genetics of sleep and sleep disorders. Cell 146, 194-207.
Sephton, C. F., et al. (2011). Identification of neuronal RNA targets of TDP-43-containing ribonucleoprotein complexes. J. Biol. Chem. 286, 1204-1215.
Sreedharan, J., et al. (2008). TDP-43 mutations in familial and sporadic amyotrophic lateral sclerosis. Science 319, 1668-1672.
Tan, C. F., et al. (2007). TDP-43 immunoreactivity in neuronal inclusions in familial amyotrophic lateral sclerosis with or without SOD1 gene mutation. Acta Neuropathol. 113, 535-542.
Tollervey, J. R., et al. (2011). Characterizing the RNA targets and position-dependent splicing regulation by TDP-43. Nat. Neurosci. 14, 452-458.
Vance, C., et al. (2009). Mutations in FUS, an RNA processing protein, cause familial amyotrophic lateral sclerosis type 6. Science 323, 1208-1211.
Voigt, A., et al. (2010). TDP-43-mediated neuron loss in vivo requires RNA-binding activity. PLoS ONE 5, e12247.
Vucic, S., et al. (2008). Cortical hyperexcitability may precede the onset of familial amyotrophic lateral sclerosis. Brain 131, 1540-1550.
Wagh, D. A., et al. (2006). Bruchpilot, a protein with homology to ELKS/CAST, is required for structural integrity and function of synaptic active zones in Drosophila. Neuron 49, 833-844.
Wang, I. F., et al. (2008). TDP-43, the signature protein of FTLD-U, is a neuronal activity-responsive factor. J. Neurochem. 105, 797-806.
Wegorzewska, I., et al. (2009). TDP-43 mutant transgenic mice develop features of ALS and frontotemporal lobar degeneration. Proc. Natl. Acad. Sci. USA 106, 18809-18814.
Wu, C. H., et al. (2012). Mutations in the profilin 1 gene cause familial amyotrophic lateral sclerosis. Nature 488, 499-503.
Xiong, W. C., et al. (1994). repo encodes a glial-specific homeo domain protein required in the Drosophila nervous system. Genes Dev. 8, 981-994.
Yu, Z., et al. (2012). Neurodegeneration-associated TDP-43 interacts with fragile X mental retardation protein (FMRP)/Staufen (STAU1) and regulates SIRT1 expression in neuronal cells. J. Biol. Chem. 287, 22560-22572.

### 9. Example 5

Amyotrophic Lateral Sclerosis (ALS) is a progressive neuromuscular disease for which there is no cure. In one aspect, disclosed herein is a Drosophila model of ALS based on TAR DNA-Binding Protein 43 (TDP-43) that recapitulates several aspects of disease pathophysiology. Using this model, a drug screening strategy was designed based on the pupal lethality phenotype induced by TDP-43 when expressed in motor neurons. In screening 1,200 FDA approved compounds, the Peroxisome Proliferator Activated Receptor (PPARγ) agonist pioglitazone was identified as neuroprotective in Drosophila. Here, it is shown that pioglitazone can rescue TDP-43 dependent locomotor dysfunction in motor neurons and glia but not in muscles. Testing additional models of ALS it was found that pioglitazone is also neuroprotective when Fused in Sarcoma (FUS), but not Superoxide Dismutase 1 (SOD1), is expressed in motor neurons. Survival analyses of TDP or FUS models showed no increase in lifespan, which is consistent with recent clinical trials. Using a pharmacogenetic approach, the data described herein indicate that the predicted Drosophila PPARγ homologs, E75 and E78 are in vivo targets of pioglitazone. Finally, using a global metabolomic approach, a set of metabolites that pioglitazone can restore in the context of TDP-43 expression in motor neurons were identified. Taken together, the data provide evidence that modulating PPARγ activity, although not effective at improving lifespan, provides a molecular target for mitigating locomotor dysfunction in TDP-43 and FUS but not SOD1 models of ALS in Drosophila. Furthermore, the data also identifies several “biomarkers” of the disease that can be useful in developing therapeutics and in future clinical trials.

### a. Introduction for Example 5

ALS is a progressive neurodegenerative disease that leads to paralysis and death within 2-5 years after diagnosis (1). It is a debilitating neuromuscular disease affecting upper and lower motor neurons for which there is no cure, and the available treatments are palliative at best. Several loci, including SOD1, TDP-43, FUS, C9ORF72 and profilin have been implicated in ALS (2-6). In particular, TDP-43 protein has been linked to a vast majority of ALS cases due to its presence in pathological aggregates. In addition to being a marker of pathology, TDP-43 can harbor disease causative mutations that lie primarily in the C terminal domain of the protein (7-11). TDP-43 is an RNA-binding protein that normally resides within the nucleus, can shuttle into the cytoplasm via a Nuclear Export Signal (NES), and harbors a prion-like domain within its C-terminus (12, 13). TDP-43's normal functions include various aspects of RNA processing such as splicing, transport and translation (14-17), with several studies providing evidence for RNA dysregulation in disease (18-20).

Evidence exists to suggest that ALS is a proteinopathy accompanied by abnormalities in diverse cellular processes including oxidative stress response, defects in ER-mitochondrial interaction, apoptosis and dysregulation of cellular metabolism among others (21-28). Findings that ALS patients exhibit hypermetabolism accompanied by dramatic weight loss have led to the hypothesis that cellular and/or systemic metabolism are involved in the onset and the progression of the disease (29, 30). Additional observations supporting this idea include studies in SOD1 mice showing that a high fat diet is protective (31) and a recent Phase 2 clinical trial that found high calorie diets to be safe and well-tolerated in patients, with an apparent improvement in lifespan (28) In another study, diabetic patients had a later onset and slower progression of ALS (32). Taken together, these data suggest that some aspects of cellular metabolism can be linked to disease progression.

To identify compounds and molecular targets with neuroprotective potential in ALS, a drug screen was performed using a Drosophila model of ALS based on TDP-43 (33-35). In screening 1,200 FDA approved compounds for their ability to rescue TDP-43 induced lethality, several antidiabetic drugs were identified, including Thiazolidinediones (TZDs), which bind with high affinity and stimulate the activity of the nuclear receptor PPARγ (36, 37). PPARs are ligand activated transcription factors belonging to the nuclear hormone receptor superfamily (38). They control several physiological processes including glucose and lipid metabolism as well as growth and differentiation. Upon ligand binding, PPARγ forms heterodimers with Retinoid X receptors (RXRs), then binds and activates the transcription of target genes involved in lipid metabolism. PPARγ is expressed in several cell types, including adipose tissues, cells of the immune system, and parts of the brain including microglia and astrocytes, the sites that contribute to anti-inflammatory response in the CNS (39). Activation of PPARγ has been shown to have anti-inflammatory and neuroprotective effects in ALS and related neurodegenerative conditions (39). The effects of pioglitazone in multiple Drosophila models of ALS, including TDP-43, FUS and SOD1, as described herein. While pioglitazone rescued TDP-43 and FUS dependent phenotypes in motor neurons, it had no effect in the SOD1 model. The protective effects of pioglitazone were observed when TDP-43 was expressed in motor neurons or glia, but not in muscles, indicating that PPARγ activation is required in the nervous system to mitigate TDP-43 toxicity. In contrast to its positive effects on locomotor function, pioglitazone did not improve, and in some cases, shortened lifespan. Using a pharmacogenetic approach, the data also indicate that E75 and E78, the predicted Drosophila homologs of PPARγ are required for mediating the neuroprotective effect of pioglitazone on TDP-43 toxicity in vivo. Metabolic profiling experiments identify a subset of metabolites that are altered in the context of TDP-43 and can be restored by pioglitazone in a variant dependent manner. Taken together, these data indicate that PPAR activation in neurons and glia is partially neuroprotective, and can restore a subset of metabolic alterations in ALS that can serve as useful biomarkers in the development of therapeutic strategies and future clinical trials.

### b. Results for Example 5

(1) Drug Screening in a Drosophila Model of ALS Based on TDP-43 Identifies Pioglitazone, a PPARγ Agonist as Neuroprotective

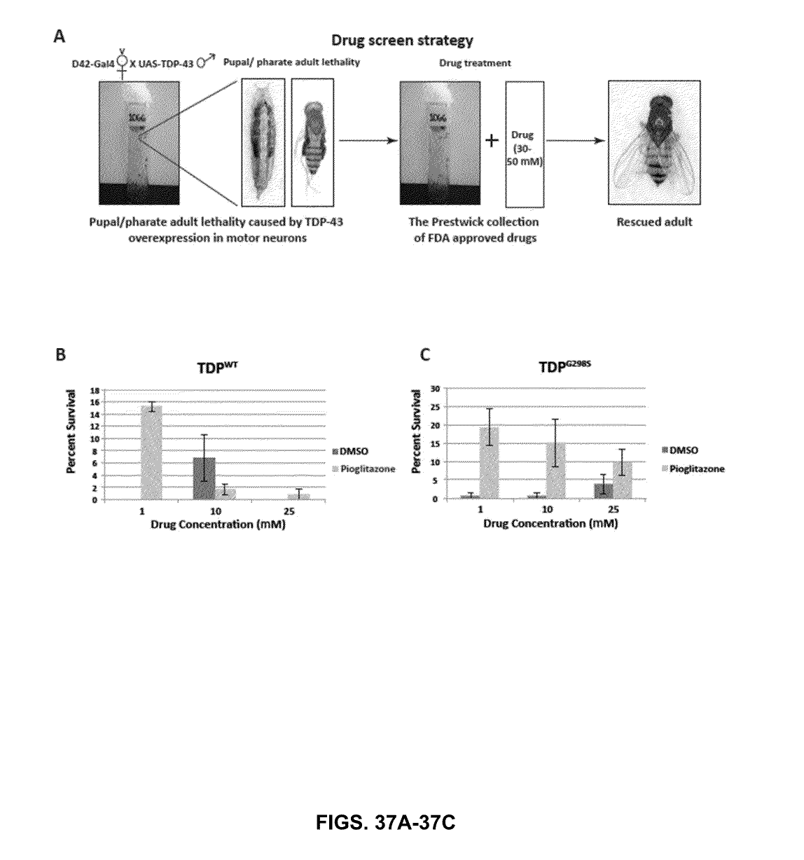

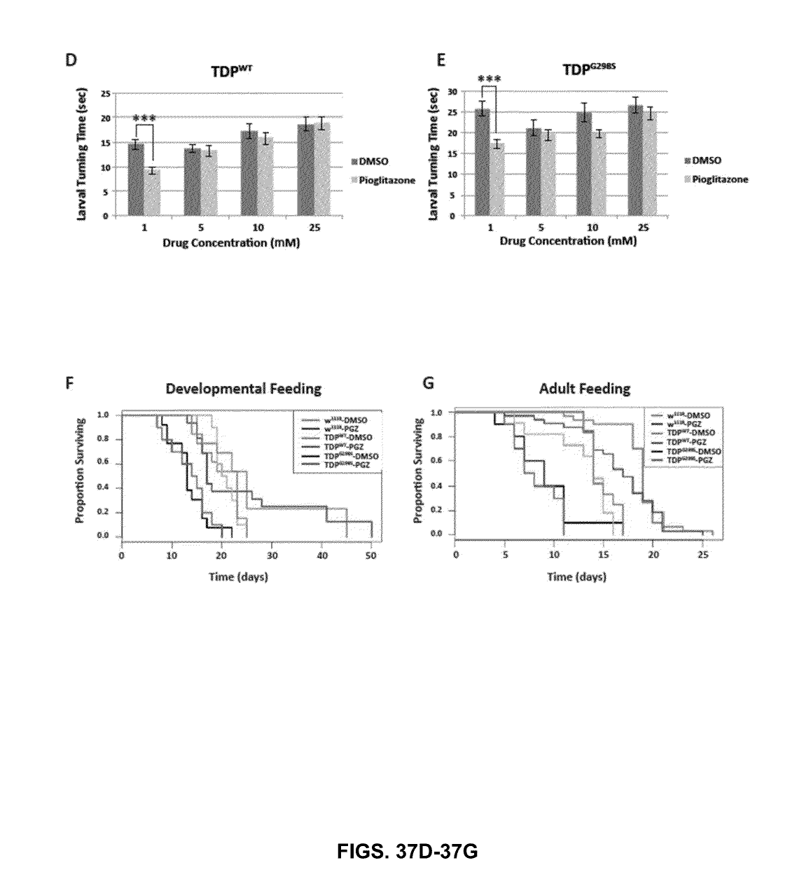

Using a Drosophila model of ALS based on TDP-43 that recapitulates several aspects of the disease pathophysiology (33, 35), a drug screen aimed at identifying FDA approved compounds with neuroprotective potential in vivo were designed (FIG. 37A). Human TDP-43, either wild type or disease-associated mutants D169G, G298S, A315T and N345K (8, 10, 40) were expressed in motor neurons using the D42-Gal4 driver (41) and it was found that this results in nearly 100% pupal lethality (FIG. 37A). Few pharate adults eclose but cannot extend their wings (FIG. 37A). This phenotype was used to screen for drug candidates with therapeutic potential as determined by their ability to rescue pupal lethality and produce viable adults with extended wings (FIG. 37A). There are several advantages to this screening strategy: 1) it is performed in vivo, 2) it is based on a robust phenotype (>95% pupal/adult pharate lethality), 3) it can lead to rapid identification of known safe drugs that are already used in humans and 4) it can inform the future development of novel small molecules with enhanced neuroprotective capabilities. In screening the Prestwick collection of 1,200 FDA-approved drugs at 30-50 μM, several antidiabetic drugs comprising different categories including thiazolidinediones, sulfonylureas and biguanides were identified. Of these, the focus was initially on the two thiazolidinediones identified in the primary screen, namely pioglitazone and troglitazone. Upon secondary screening, pioglitazone was determined to be more potent and selected for further studies.

To assess the neuroprotective effect of pioglitazone, two transgenic lines (TDPWT and TDPG298S) expressing comparable levels of TDP-43 were chosen. Larvae expressing TDP-43 in motor neurons (D42>TDP-43) were raised on fly food containing different amounts of drug (1, 10 and 25 μM, see Materials and Methods for details) or Dimethyl Sulfoxide (DMSO) as vehicle control (FIG. 37B, C). As seen in FIGS. 37B and C, the data indicate that although there was no clear dose response in this assay, lower drug concentrations exhibited higher neuroprotective potential and pupal lethality caused by the expression of either TDPWT or TDPG298S in motor neurons was best rescued by 1 M pioglitazone (see also Table 5). Taken together, these data indicate that pioglitazone mitigates TDP-43 dependent pupal lethality.

TABLE 5
Summary of rescue of lethality by pioglitazone. Genotype, drug
concentration and experimental conditions, as indicated. Average percent
rescue was calculated as (number of rescued adults/total number of pupae) ×
100%. Notably, lower drug concentrations led to better rescue.

| | TDP^{WT} | | | | TDP^{G298S} | | | |
|---|---|---|---|---|---|---|---|---|
| PGZ | DMSO | | PGZ | | DMSO | | PGZ | |
| conc | Av. % | | Av. % | | Av. % | | Av. % | |
| (μM) | rescue | SEM | rescue | SEM | rescue | SEM | rescue | SEM |
| 1 | 0 | 0 | 15.2 | 0.8 | 0.7 | 0.7 | 19.4 | 5.0 |
| 10 | 6.8 | 3.8 | 1.7 | 0.8 | 0.7 | 0.7 | 15.0 | 6.5 |
| 25 | 0 | 0 | 0.8 | 0.7 | 3.8 | 2.7 | 9.7 | 3.6 |

PGZ—pioglitazone containing food,
DMSO—vehicle control,
SEM—Standard error of mean.

(2) Larval Locomotor Deficits Caused by Tdp-43 Expression in Motor Neurons are Rescued by Pioglitazone

Next an ideal drug concentration was determined using a more sensitive and disease relevant assay that measures neuromuscular coordination, namely larval turning (see Materials and Methods for details). Larvae expressing TDPWT or TDPG298S in motor neurons (D42>TDP-43) were raised on food containing either 1, 5, 10, 25 μM pioglitazone or DMSO and assayed for their locomotor ability using larval turning assays. As shown in FIGS. 37D and E, pioglitazone significantly improved larval locomotion at 1 μM for both TDPWT (9.2+/−0.7 sec on pioglitazone compared to 14.5+/−1 sec on DMSO, Pvalue=0.7E-4) and TDPG298S (17.3+/−1.1 sec on pioglitazone compared to 25.8+/−1.8 sec on DMSO, Pvalue=0.2E-3). No rescue was observed at higher drug concentrations and lower doses were not tested. 1 μM pioglitazone had no effect on larval turning time in wild-type control larvae (D42>w1118) indicating that the drug effect is specific to TDP-43 dependent phenotypes. Finally, the neuroprotective effect of pioglitazone was not due to a reduction in TDP-43 protein levels as determined by Western blot. These results indicate that the ideal concentration for rescuing TDP-43 dependent larval locomotor defects in the range tested, is 1 μM pioglitazone, which was used for all subsequent experiments.

(3) Pioglitazone Exerts No Protective Effect on Lifespan in the Context of TDP-43 Expression in Motor Neurons

Next, the study tested whether pioglitazonecan also improve the decrease in lifespan caused by TDPWT or TDPG298S expression in motor neurons. To this end, larvae expressing either TDPWT or TDPG298S in motor neurons on 1 μM pioglitazone were raised and it was found that the rescued adults had a similar lifespan to larvae raised on DMSO containing food (FIG. 37F). Since obtaining adults on DMSO is very difficult due to the >95% pupal lethality caused by TDP-43, these studies were performed with 10-15 adult flies (“escapers”) only. Thus although 1 μM pioglitazone can improve locomotor function when administered during development, it has no effect on the TDP-43 dependent decrease in adult lifespan. To determine whether pioglitazone can be effective “after disease onset”, adult D42>TDP-43 “escapers” raised on normal food (see Materials and Methods for details) were fed either 1 μM pioglitazone or DMSO containing food after eclosion. As with the developmental feeding experiment, it was found that pioglitazone has no significant effect on lifespan (FIG. 37G) at least at the 1 μM concentration, which was determined to be optimal for rescuing larval locomotor defects (FIG. 37D, E). These data are consistent with recent clinical trials in which pioglitazone failed to show improvement in ALSFRS-R scores, a well-established clinical measure of disease progression (42). These results indicate that while pioglitazone is effective in improving locomotor function when administered during development it has no effect on lifespan regardless of whether it is provided throughout development or “after disease onset”.

(4) Glial Toxicity Caused by TDP-43 is Partially Mitigated by Pioglitazone

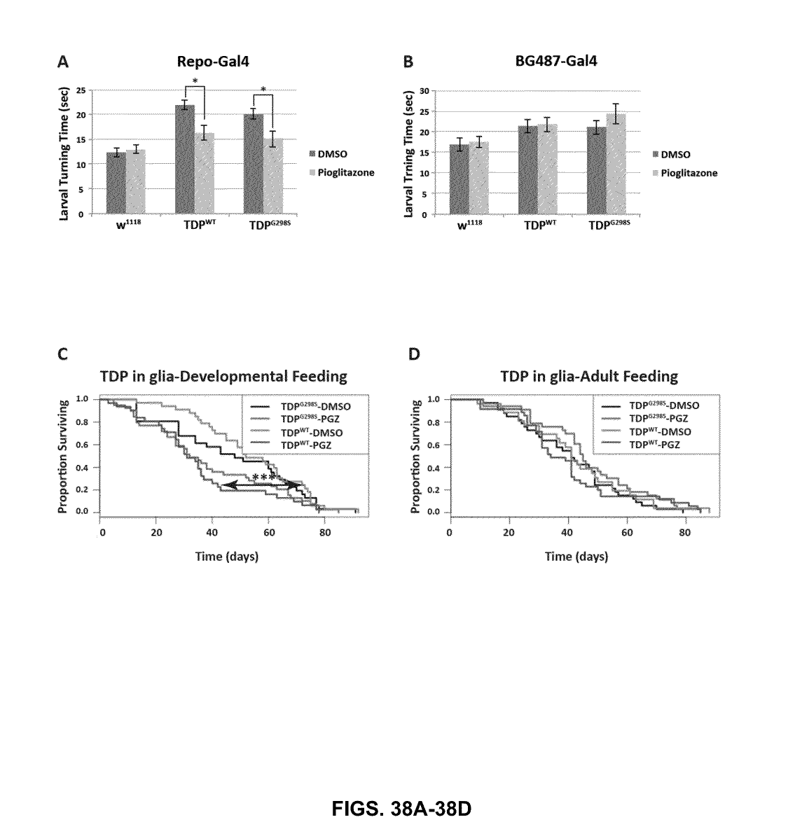

Glial and muscle cells have been previously implicated in ALS pathophysiology (43, 44). It has been shown that TDP-43 expression in glial cells results in cell autonomous locomotor defects and abnormalities in synaptic protein distribution at the neuromuscular junction (35). To determine whether pioglitazone can also mitigate glial toxicity, turning assays on larvae expressing TDP-43 was performed using the pan-glial Repo-Gal4 driver. As shown in FIG. 38A, these experiments indicate that 1 μM pioglitazone rescues locomotor function abnormalities caused by TDP-43 expression in glia compared to both DMSO and w1118 controls. These data demonstrate that pioglitazone exerts neuroprotective effects on TDP-43 induced glial toxicity as it does in motor neurons.

Next, the study tested whether 1 μM pioglitazone can also increase the lifespan of adult flies expressing TDP-43 in glia and raised on pioglitazone containing food throughout development. These experiments showed no difference in lifespan for TDPG298S, while TDPWT expressing flies lived significantly less on pioglitazone compared to DMSO (FIG. 38C). An increase in hazard ratio for mortality was found in ALS patients who were administered pioglitazone (42). This is in contrast to TDPWT expression in motor neurons, where there was no difference in lifespan between drug and DMSO fed groups (FIG. 37F). Furthermore, when pioglitazone was administered to adults expressing TDP-43 in glia, no difference in lifespan was found between the drug-treated flies and DMSO controls (FIG. 38D). These data indicate that although the drug can improve locomotor function caused by TDP-43 toxicity in glial cells, it has either no effect or can be detrimental to lifespan in flies as it is in humans.

(5) Locomotor Defects Caused by TDP-43 in Muscles are not Rescued by Pioglitazone

Next, the study tested whether pioglitazone can exert beneficial effects on muscle dependent locomotor phenotypes caused by TDPWT or TDPG298S expression in larvae using the muscle specific Gal4 driver, BG487 (45). In contrast to the results in motor neurons and glia, it was found that 1 μM pioglitazone did not rescue TDP-43 dependent larval turning defects when expressed in larval muscles (FIG. 38B). Because BG487-Gal4 drives expression only in larval body wall muscles 6/7 in an anterior to posterior gradient, the effect of pioglitazone in the context of mhc-Gal4, which drives expression strongly in all larval muscles was also tested and obtained similar results. These data indicate that pioglitazone cannot rescue TDP-43 dependent locomotor defects caused by muscle specific expression. Although it is possible that a different concentration is needed for pioglitazone to mitigate TDP-43 induced toxicity in muscles, the data indicate that the protective effects of pioglitazone can be restricted to the nervous system.

(6) FUS Dependent Toxicity in Motor Neurons is Partially Mitigated by Pioglitazone

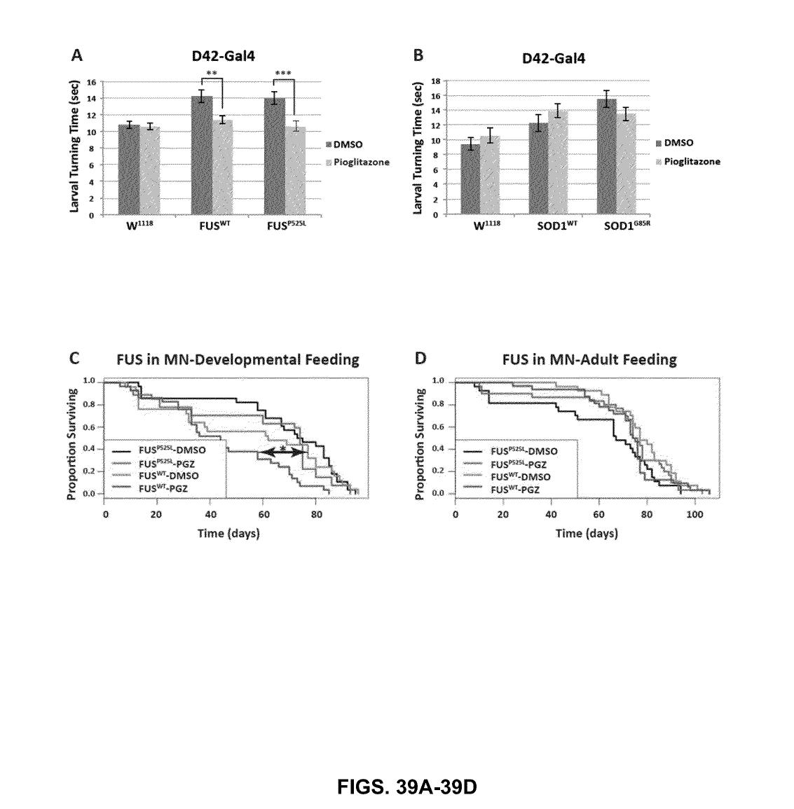

Having established that pioglitazone rescues several aspects of TDP-43 mediated toxicity in motor neurons and glia, whether it could also rescue toxicity in a Drosophila model of ALS based on human FUS (46), another RNA binding protein linked to ALS that shares some functional aspects with TDP-43 (47) was tested. As shown in FIG. 39A expression of both human FUSWT and disease associated mutant FUSP525L in motor neurons resulted in impaired larval locomotion, which is rescued by 1 μM pioglitazone. These data indicate that the beneficial effect of pioglitazone extends to FUS dependent toxicity in motor neurons at least in regards to locomotor dysfunction.

The study also tested whether pioglitazone can improve lifespan in adults expressing FUS in motor neurons. As with Repo>TDP-43 experiments (FIG. 38C), developmental feeding of pioglitazone resulted in a significantly shorter lifespan for FUSWT flies, but not FUSP525L flies (FIG. 39C). No difference was observed between the two variants following adult feeding (FIG. 39D). Overall these data indicate that although pioglitazone can rescue some aspects of the disease, it is not effective in improving lifespan in adult flies.

(7) Pioglitazone is not Protective in a Drosophila Model of ALS Based on SOD1

Since pioglitazone was previously shown to be protective in a SOD1 mouse model of ALS (48, 49), whether it could also mitigate locomotor defects in a Drosophila model was tested (50). These experiments showed that in contrast to the results with TDP-43 and FUS models, pioglitazone does not rescue the larval turning phenotype caused by the expression of SOD1G85R mutant in motor neurons (FIG. 3B). Unlike TDP-43 and FUS, the expression of wild-type human SOD1 (SOD1WT) in motor neurons does not result in a larval turning phenotype compared to w1118 controls (FIG. 39B). These findings are in contrast to previous studies in mice where oral administration of pioglitazone to animals expressing SOD1G93A showed both improved locomotion and increased lifespan (48, 49). Although a different SOD1 mutation was used than in the mouse study, SOD1G85R and SOD1G93A mice share several phenotypic features, including motor neuron degeneration, paralysis of fore- and hindlimbs, and muscle atrophy, leading to rapid progression of the disease (51, 52). Taken together these results indicate that pioglitazone rescues larval locomotion defects caused by motor neuron expression of TDP-43 and FUS, but not SOD1 and indicate that distinct molecular mechanisms can underlie different sub-types of motor neuron disease.

(8) PPAR Acts as the Molecular Target of Pioglitazone In Vivo, in DROSOPHILA

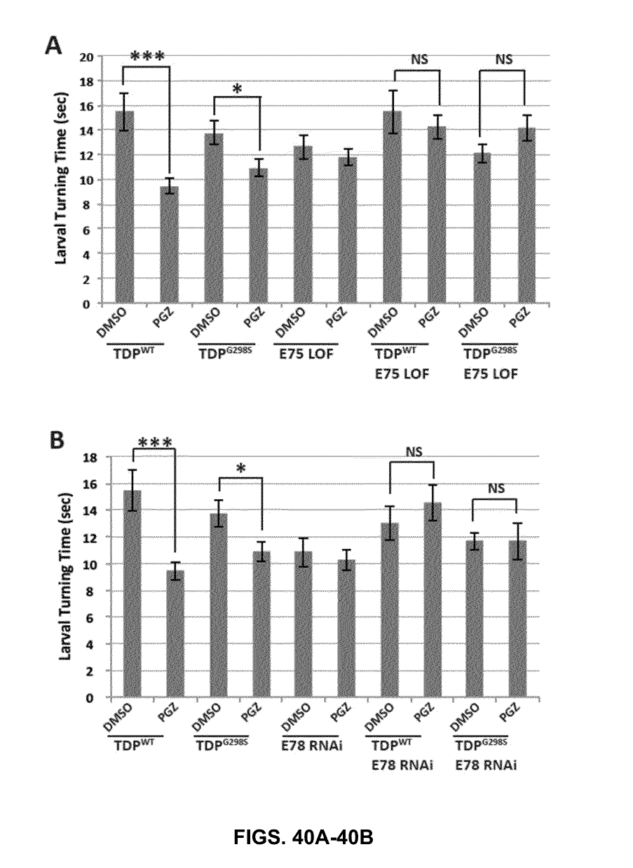

In humans, pioglitazone activates the nuclear receptor PPARγ, resulting in transcriptional modulation of factors that reduce insulin resistance (53). In the nervous system, PPARγ exerts neuroprotection by reducing inflammation (39, 54). Recently, pioglitazone shown to directly bind mitochondrial proteins suggesting that it can act on additional targets in vivo (55, 56). Although Drosophila PPARγ has not been well characterized, there are two predicted homologs, namely E75 and E78 that belong to the nuclear receptor superfamily and are most similar in sequence to the human REV-ERBA receptor, a member of the same subfamily as nuclear PPARs (57). To determine whether pioglitazone exerts its neuroprotective activities in Drosophila by activating PPARγ, a pharmacogenetic approach was taken. Indeed, if PPARγ is required for pioglitazone's action, reducing the receptor's expression in the context of TDP-43 can render motor neurons insensitive to the beneficial effects of the drug. To test this, a loss of function allele was used for E75 (Eip75B) in the context of TDPWT or TDPG298S and it was found that when E75 expression was reduced by 50%, pioglitazone no longer rescues TDP-43 dependent larval turning defects (FIG. 40A). Similar results were obtained when E78 was knocked down by RNAi in the context of TDP-43 (FIG. 40B). Knock-down was confirmed by semiquantitative PCR that showed a reduction of ˜40% in transcript levels compared to controls. These data indicate that PPARγ is the primary in vivo target of pioglitazone in the fly and are consistent with the notion that activating PPARγ mitigates aspects of TDP-43 dependent toxicity.

(9) Pioglitazone Restores a Subset of Metabolites Dysregulated in the Context of TDP-43 Proteinopathy

To gain insight into the mechanism by which pioglitazone is neuroprotective, a global metabolomic approach was taken using larvae expressing TDP-43 in motor neurons (D42>TDP-43) and D42>w1118 controls raised on either 1 μM pioglitazone, DMSO or regular food. To determine specific metabolites that were restored by pioglitazone in the context of TDPWT, the metabolic profiles of D42>TDPWT larvae raised on drug food versus DMSO were compared and 111 alterations were found among a total of 572 metabolites detected in the samples. Next, the study tested which of the 111 pioglitazone specific changes were also caused by TDPWT expression as determined by comparing the metabolic profiles of TDP-43 expressing larvae and D42>w1118 larvae raised on regular food. This led to the identification of 28 metabolites altered by both pioglitazone and TDPWT (Table 2). Of these, 14 metabolites were altered in opposite direction compared to TDPWT, consistent with a restoration by pioglitazone while 8 others appeared to be similarly affected by pioglitazone and TDPWT, indicating that pioglitazone does not further affect these biochemicals. In addition, 6 metabolites further altered by pioglitazone in the same direction as that of TDP-43 were found, consistent with a potential worsening effect. Among the 28 metabolites some were also affected by DMSO alone as determined by comparing the metabolic profiles of D42>TDPWT larvae raised on DMSO and D42>TDPWT larvae raised on regular food.

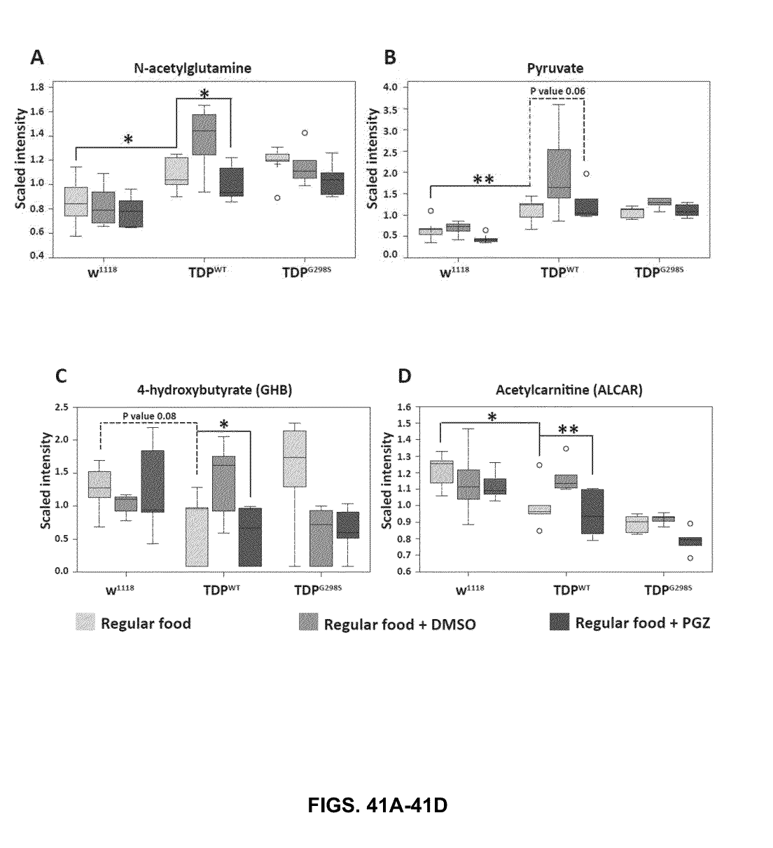

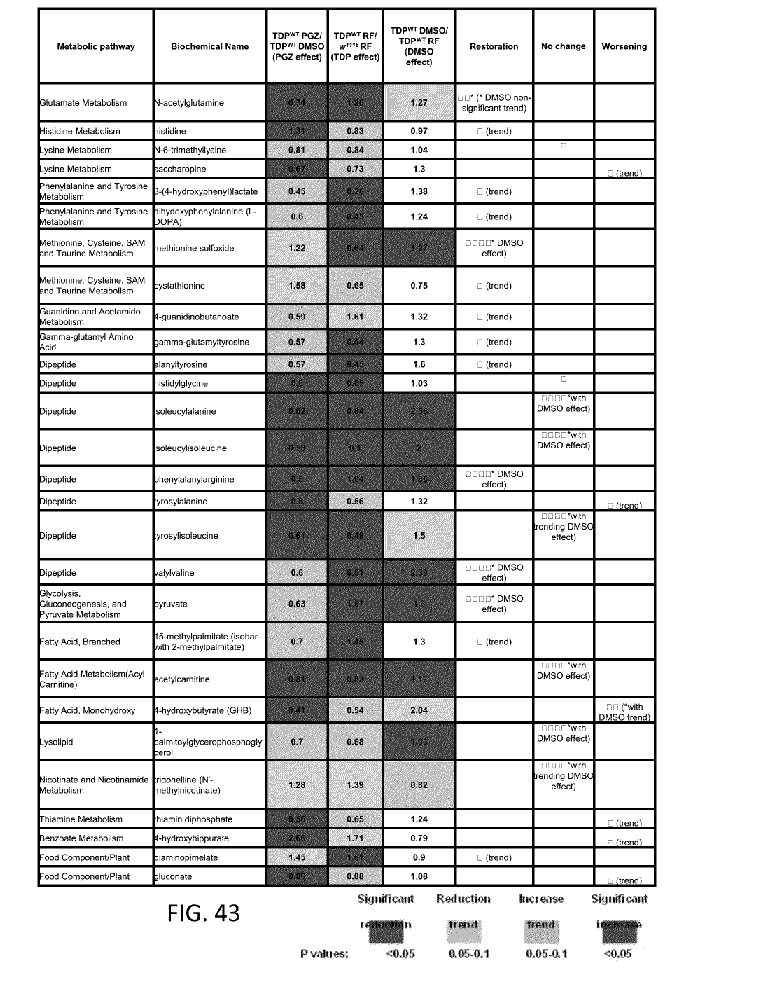

The sole significantly restored metabolite was N-acetylglutamine, which was increased in D42>TDPWT larvae versus D42>w1118 on regular food (1.26, Pvalue<0.05) and reduced by pioglitazone (in D42>TDPWT larvae on pioglitazone versus DMSO, 0.74, Pvalue<0.05, FIG. 41A and FIG. 43). There was a trend towards higher N-acetylglutamine on DMSO compared to regular food (1.27, Pvalue=0.06) however, since pioglitazone significantly reduced its levels, the effect of DMSO was considered to be negligible. Increased N-acetylglutamine, a stable form of glutamine indicates alterations in glutamate metabolism, consistent with reports of glutamate excitotoxicity in ALS (58). Pioglitazone also restored the levels of phenylalanylarginine (PAA), a dipepetide found to be elevated in D42>TDPWT larvae compared to D42>w1118 on regular food; however, DMSO had a significant effect on this metabolite, thus rescue was not definitely ascertained in this case (see FIG. 43). Increased levels of PAA indicate defects in protein turnover, which are consistent with alterations in protein clearance known to accompany TDP-43 proteinopathy (59). Several metabolites showed trends towards restoration but did not reach statistical significance. Among these, pyruvate, the end product of glycolysis and a key metabolite at the intersection between several metabolic pathways was significantly increased in both D42>TDPWT and D42>TDPG298S larvae (1.67 and 1.6, compared to D42>w1118 on regular food, respectively) and was slightly but not significantly reduced in D42>TDPWT raised on pioglitazone compared to DMSO (0.68, see FIG. 41B and FIG. 43).

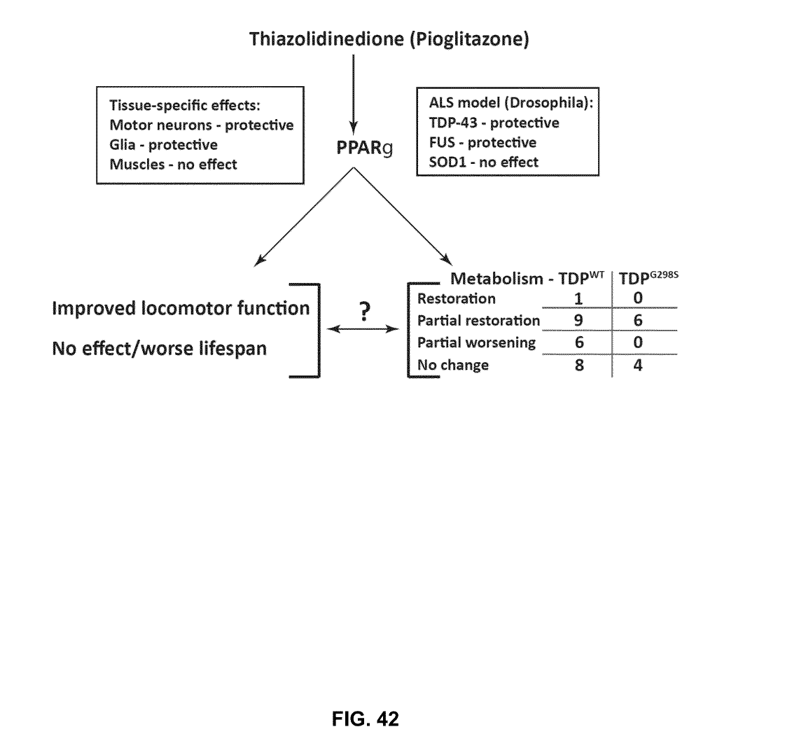

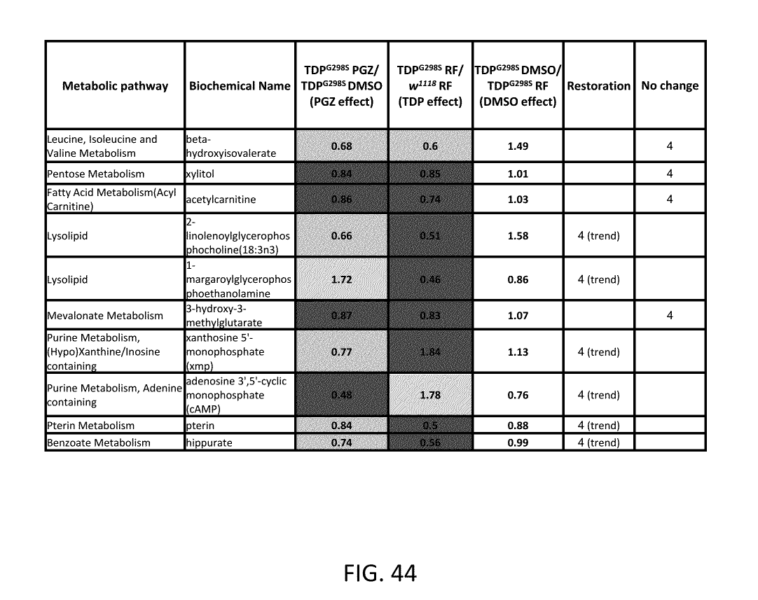

Saccharopine, an intermediate in Lysine metabolism, while it trends low in D42>TDPWT larvae (0.73 compared to D42>w1118 on regular food, Pvalue=0.08), it was significantly reduced on pioglitazone (0.67 compared to DMSO, FIG. 43). Similarly, 4-hydroxybutyrate (GHB), a ketone body was significantly reduced by pioglitazone (0.41, Pvalue=0.04) from trending low in TDPWT (0.54, Pvalue=0.08, FIG. 5C and FIG. 43). SOD1 mice on ketogenic diet exhibited slower disease progression, which can be attributed to the ability of ketone bodies to generate energy (60). These data indicate that some metabolites worsen in the context of pioglitazone, which can explain the deleterious effect of pioglitazone on lifespan. Additional metabolites of interest include acetylcarnitine (ALCAR), a precursor to carnitine, which was significantly reduced in the context of TDP-43 (Joardar and Zarnescu, unpublished observations) and remained low in the context of pioglitazone (FIG. 41D and FIG. 43). Given carnitine's role in transporting fatty acids into the mitochondria for breakdown, these data indicate a TDP-43 dependent decrease in lipid beta oxidation. Notably, while there were some trends towards restoration in D42>TDPG298S larvae, none of the metabolite rescues were statistically significant (see FIG. 44). These findings are consistent with distinct mechanisms underlying TDPWT versus mutant TDPG298S toxicity at the cellular level (FIG. 42). Overall, the vast majority of alterations in amino acid or lipid metabolism was not restored by pioglitazone or, was, in some cases worsened, consistent with the drug's ability to rescue some but not all TDP-43 dependent phenotypes in Drosophila.

### c. Discussion for Example 5

ALS is the third most common form of neurodegeneration following Alzheimer's and Parkinson's diseases (61). Although Riluzole is approved for ALS patients, its benefits are marginal and at this time there are no known effective treatments for the disease. There have been several efforts to design therapeutics using the SOD1 mouse, the most commonly used animal model of ALS. However, despite promising pre-clinical results, these candidate drugs have been disappointing in humans (62, 63). To address this significant issue, efforts are being made to develop other animal models of ALS that can be used not only to identify phenotypes and “early biomarkers” of the disease, but also will be useful in drug screens for therapeutic purposes. A Drosophila model of ALS has been generated based on TDP-43, which recapitulates several aspects of the human disease including locomotor dysfunction and reduced lifespan (33, 35). Here, using this model, it was shown that the antidiabetic drug pioglitazone acts as a neuroprotectant for aspects of TDP-43 proteinopathy by activating the putative Drosophila PPARγ homologs E75 and E78. It was also shown that pioglitazone mitigates FUS but not SOD1 dependent toxicity in Drosophila, consistent with previous published work showing that distinct mechanisms are likely at work in the context of these different models of ALS (64). Pioglitazone did not improve the lifespan of TDP-43 expressing flies, when administered either during development, or after “disease onset”, which is consistent with results from recent clinical trials (42, 65). Except for the possibility that different drug concentrations may be needed it remains unclear why pioglitazone is protective in mouse but not fly SOD1 models and, in retrospect, given the similarities between the effect of pioglitazone in Drosophila models of ALS and humans, the fly appears to be a more accurate predictor of clinical trial outcomes.

It is tempting to speculate that the predictive power of the Drosophila model may lie in the tools that enable motor neuronal versus glial versus muscle specific expression of the toxic TDP-43 protein. The results described herein show that pioglitazone mitigates neuronal and glial TDP-43 dependent toxicity but has no effect on the locomotor dysfunction caused by muscle specific expression of TDP-43. This type of knowledge is easily obtainable in the fly model and can provide helpful information about cell autonomous versus non-autonomous effects as well as the efficacy of candidate drugs in different tissues of interest. While it was shown that pioglitazone reduces inflammation in the glia, its effects in neurons or muscles have not been studied in the mouse prior to human trials (48). The results indicate that the protective effects of pioglitazone are specific to the nervous system and were not observed in muscles, at least within the limits of the experimental conditions (i.e., tissue specific levels of expression and drug concentration) described herein. These findings indicate that future preclinical studies can benefit from testing candidate therapies in multiple disease models in which tissue specificity and several phenotypic outcomes are easily ascertained.

Pioglitazone has been originally developed for the treatment of type 2 diabetes as PPARγ activation in the liver improves glucose metabolism systemically (66). In the nervous system, activation of the nuclear hormone receptor PPARγ has been shown to have anti-inflammatory and neuroprotective effects (39, 54). In the model used herein, pioglitazone was found to restore a rather limited set of metabolites altered in a TDP-43 dependent manner (see FIG. 42). Altered cellular metabolism has previously been implicated in ALS pathophysiology and ALS patients exhibit signs of hypermetabolism (28-32, 67). Notably, the fly model described herein also showed signs of hypermetabolism including an increase in pyruvate, a key metabolite linking glucose metabolism to the TCA cycle. Additionally the ketone body GHB is reduced in the context of TDPWT, consistent with a clinical study showing that a ketogenic diet slowed ALS disease progression (60). Given the similarities between the metabolic profile of the Drosophila model and human samples, it will be interesting in the future to design therapeutic approaches aimed at restoring these common metabolic changes using nutritional supplementation.

In summary, the data described herein show the potential of using the fly model of ALS as a rapid and efficacious system for drug screening in vivo. The results using FUS and SOD1 fly models of ALS indicate that pioglitazone is effective in mitigating some, but not all forms of the disease, which indicates that stratification of patient populations should be considered in future clinical trials. The primary endpoint tested in past clinical trials, namely lifespan, was not improved by pioglitazone, which is consistent with the data in Drosophila. However, the developmental and adult feeding experiments clearly demonstrate that locomotor function is improved indicating that PPARγ remains a molecular target with therapeutic potential, perhaps in combination with other strategies based on restoring the metabolic alterations caused by TDP-43 in the nervous system.

### 10. Materials and Methods for Example 5

### a. Drosophila Genetics

All Drosophila stocks and crosses were maintained on standard yeast/cornmeal/molasses food at 25° C. Transgenic flies expressing human TDP-43 variants with C-terminal YFP tags were generated described herein (33, 35). GMR Gal4, D42 Gal4, Repo-Gal4, BG487-Gal4 or mhc-Gal4 were used to drive expression in different tissues using the GAL4-UAS system (68) and were obtained from the Bloomington Stock Center together with Eip75B/TM6B, P{Ubi-GFP.S65T}PAD2, Tb, y1 v1; P{TRiP.JF02258}attP2, w1118; P{UAS-hSOD1.G85R}2a P{UAS-hSOD1.G85R}2b; P{UAS-hSOD1.G85R}3a P{UAS-hSOD1.G85R}3b, and w1118; P{UAS-hSOD1}16.2. w1118; UAS-FUSWT and w1118; UAS-FUSP525L transgenics were gifts from Brian McCabe. For genetic controls, w1118 was crossed with the appropriate Gal4 driver.

### b. Drug Screen

UAS TDP-43 males were crossed with D42-Gal4 female virgins on fly food containing either DMSO or pioglitazone. For DMSO controls, the same volume of DMSO as the corresponding drug concentration was added. Bromophenol blue was added to a final concentration of approximately 0.02% to ensure homogeneity. Crosses were made on drug food with 3 female virgins and 2 males in each vial, and were maintained at 25° C. unless noted. The parents were discarded after 5-7 days then the vials were screened for adult progeny with straight wings from day 14 to day 25. All adults were screened for TDP-43 expression by visualizing the YFP tag. Total number of pupae was counted on day 25. Percent survival was calculated using the formula (total number of straight-winged adults/total number of pupae)×100. All experiments were performed in triplicate.

### c. Larval Turning Assay

Assays were performed as described herein (33, 35). Briefly, wandering third instar larvae were placed on room temperature grape juice plates, allowed to acclimate then gently turned ventral side up. The time required for larvae to flip back to dorsal side up and start moving forward was noted as the larval turning time. At least 30 larvae per genotype and condition were tested.

### d. Lifespan Analysis with Pioglitazone

For developmental feeding experiments, crosses were made on drug food at 25° C. Newly enclosed flies were separated by sex and housed in separate vials with no more than 4 flies per vial at 25° C. For adult feeding experiments, crosses were made on regular food at 22° C. Immediately after eclosion, flies were separated by sex and placed in fresh vials with drug food at 25° C. Survival plots were generated using the survival and Hmisc packages in R (R Development Core Team, 2013) and Rstudio (R Studio, Inc. Boston, Mass., USA) software. Statistical analysis was done using the log-rank test in R.

### e. Metabolomics

D42 Gal4 virgin females were crossed with UAS TDP-43 YFP or w1118 males and raised on regular food (RF), DMSO or 1 μM Pioglitazone (PGZ) containing food. 50-60 third instar larvae (approximately 50-60 mg) were collected and flash frozen in liquid nitrogen. Samples were made in 5 replicates. Global metabolomic study was performed following standard protocols by Metabolon, INC (Durham, N.C.). Briefly, protein samples were divided into four fractions: one for analysis by UPLC-MS/MS with positive ion mode electrospray ionization, one for analysis by UPLC-MS/MS with negative ion mode electrospray ionization, one for analysis by GC-MS, and one sample was reserved for backup. Raw data was extracted, peak-identified and QC processed using Metabolon's hardware and software. Compounds were identified by comparison to library entries of purified standards or recurrent unknown entities. Peaks were quantified using area-under-the-curve. Following normalization to Bradford protein concentration data were log transformed. Missing values for a given metabolite were imputed with the minimum observed value for each compound based on the assumption that they were below the limit of instrument detection sensitivity. For studies spanning multiple days, a data normalization step was performed to correct variation resulting from instrument inter-day tuning differences.

### f. Western Blotting

Western blotting was done following standard protocol. Briefly, protein samples (whole larvae crushed in 2× Laemmli Buffer) were resolved on 4-20% SDS-PAGE gradient precast gels (BioRad) and then transferred to a PVDF membrane (Millipore). Following blocking in 5% fat-free milk in 1×TBST, the following primary antibodies were used for different experiments at 4° C. overnight: rabbit anti-GFP (Invitrogen) at 1:6,000, and mouse anti-tubulin β clone KMX-1 (Millipore) at 1:1,000. Secondary antibodies used were: goat anti-rabbit-conjugated-HRP (Thermo Scientific) at 1:1,000 or goat anti-mouse-conjugated-HRP (Thermo Scientific) at 1:1,000. Proteins were detected using SuperSignal West Femto Substrate (Thermo Scientific), and quantified using LICOR Image Studio Lite software.

### g. Semi-Quantitative PCR

Total RNA was prepared from fly heads (GMR Gal4>E78RNAi) using RNeasy Kit (Qiagen) with on-column DNAse treatment. First strand cDNA synthesis was performed with a Superscript III cDNA synthesis Kit (Invitrogen). Semi-quantitative PCR reactions were performed by normalizing E78 to GAPDH levels. Primers used were:

(forward)
(SEQ ID NO: 2)
TTTGGAGCGCTGCGAAGTTG
and
(reverse)
(SEQ ID NO: 3)
AGCTGTGGTTCCGTGTTG.

### h. Statistical Analyses

Student's T test was used for statistical analysis of larval turning experiments and Log-rank test was used for survival analyses. For metabolomics standard statistical analyses were performed in ArrayStudio on log transformed data. For those analyses not standard in ArrayStudio, the programs R (cran.r-project.org/) or JMP (jmp.com) were used. Welch's two-sample t-test and matched pairs t-test were used to compute p-values. False discovery rates were calculated as described (69). ANOVA contrasts were used to identify biochemicals that differed significantly between experimental groups. Analysis by two-way ANOVA identified biochemicals exhibiting significant interaction and main effects for experimental parameters of genotype and treatment.

### 11. References for Example 5

Sreedharan, J. and Brown, R. H., Jr. (2013) Amyotrophic lateral sclerosis: Problems and prospects. Ann Neurol, 74, 309-316.
Alexander, M. D., Traynor, B. J., Miller, N., Corr, B., Frost, E., McQuaid, S., Brett, F. M., Green, A. and Hardiman, O. (2002) “True” sporadic ALS associated with a novel SOD-1 mutation. Ann Neurol, 52, 680-683.
Lagier-Tourenne, C. and Cleveland, D. W. (2009) Rethinking ALS: the FUS about TDP-43. Cell, 136, 1001-1004.
Renton, A. E., Majounie, E., Waite, A., Simon-Sanchez, J., Rollinson, S., Gibbs, J. R., Schymick, J. C., Laaksovirta, H., van Swieten, J. C., Myllykangas, L. et al. (2011) A hexanucleotide repeat expansion in C9ORF72 is the cause of chromosome 9p21-linked ALS-FTD. Neuron, 72, 257-268.
Wu, C. H., Fallini, C., Ticozzi, N., Keagle, P. J., Sapp, P. C., Piotrowska, K., Lowe, P., Koppers, M., McKenna-Yasek, D., Baron, D. M. et al. (2012) Mutations in the profilin 1 gene cause familial amyotrophic lateral sclerosis. Nature.
Kwiatkowski, T. J., Jr., Bosco, D. A., Leclerc, A. L., Tamrazian, E., Vanderburg, C. R., Russ, C., Davis, A., Gilchrist, J., Kasarskis, E. J., Munsat, T. et al. (2009) Mutations in the FUS/TLS gene on chromosome 16 cause familial amyotrophic lateral sclerosis. Science, 323, 1205-1208.
Janssens, J. and Van Broeckhoven, C. (2013) Pathological mechanisms underlying TDP-43 driven neurodegeneration in FTLD-ALS spectrum disorders. Human molecular genetics, 22, R77-87.
Kabashi, E., Valdmanis, P. N., Dion, P., Spiegelman, D., McConkey, B. J., Vande Velde, C., Bouchard, J. P., Lacomblez, L., Pochigaeva, K., Salachas, F. et al. (2008) TARDBP mutations in individuals with sporadic and familial amyotrophic lateral sclerosis. Nat Genet, 40, 572-574.
Neumann, M., Sampathu, D. M., Kwong, L. K., Truax, A. C., Micsenyi, M. C., Chou, T. T., Bruce, J., Schuck, T., Grossman, M., Clark, C. M. et al. (2006) Ubiquitinated TDP-43 in frontotemporal lobar degeneration and amyotrophic lateral sclerosis. Science, 314, 130-133.
Sreedharan, J., Blair, I. P., Tripathi, V. B., Hu, X., Vance, C., Rogelj, B., Ackerley, S., Duman, J. C., Williams, K. L., Buratti, E. et al. (2008) TDP-43 mutations in familial and sporadic amyotrophic lateral sclerosis. Science, 319, 1668-1672.
Van Deerlin, V. M., Leverenz, J. B., Bekris, L. M., Bird, T. D., Yuan, W., Elman, L. B., Clay, D., Wood, E. M., Chen-Plotkin, A. S., Martinez-Lage, M. et al. (2008) TARDBP mutations in amyotrophic lateral sclerosis with TDP-43 neuropathology: a genetic and histopathological analysis. Lancet Neurol, 7, 409-416.
King, O. D., Gitler, A. D. and Shorter, J. (2012) The tip of the iceberg: RNA-binding proteins with prion-like domains in neurodegenerative disease. Brain research, 1462, 61-80.
Ou, S. H., Wu, F., Harrich, D., Garcia-Martinez, L. F. and Gaynor, R. B. (1995) Cloning and characterization of a novel cellular protein, TDP-43, that binds to human immunodeficiency virus type 1 TAR DNA sequence motifs. J Virol, 69, 3584-3596.
Buratti, E. and Baralle, F. E. (2001) Characterization and functional implications of the RNA binding properties of nuclear factor TDP-43, a novel splicing regulator of CFTR exon 9. J Biol Chem, 276, 36337-36343.
Lee, E. B., Lee, V. M. and Trojanowski, J. Q. (2012) Gains or losses: molecular mechanisms of TDP43-mediated neurodegeneration. Nature reviews. Neuroscience, 13, 38-50.
Polymenidou, M., Lagier-Tourenne, C., Hutt, K. R., Huelga, S. C., Moran, J., Liang, T. Y., Ling, S. C., Sun, E., Wancewicz, E., Mazur, C. et al. (2011) Long pre-mRNA depletion and RNA missplicing contribute to neuronal vulnerability from loss of TDP-43. Nature neuroscience, 14, 459-468.
Tollervey, J. R., Curk, T., Rogelj, B., Briese, M., Cereda, M., Kayikci, M., Konig, J., Hortobagyi, T., Nishimura, A. L., Zupunski, V. et al. (2011) Characterizing the RNA targets and position-dependent splicing regulation by TDP-43. Nature neuroscience, 14, 452-458.
Colombrita, C., Onesto, E., Tiloca, C., Ticozzi, N., Silani, V. and Ratti, A. (2011) RNA-binding proteins and RNA metabolism: a new scenario in the pathogenesis of Amyotrophic lateral sclerosis. Arch Ital Biol, 149, 83-99.
Voigt, A., Herholz, D., Fiesel, F. C., Kaur, K., Muller, D., Karsten, P., Weber, S. S., Kahle, P. J., Marquardt, T. and Schulz, J. B. (2010) TDP-43-Mediated Neuron Loss In Vivo Requires RNA-Binding Activity. PLoS One, 5.
Ling, S. C., Polymenidou, M. and Cleveland, D. W. (2013) Converging mechanisms in ALS and FTD: disrupted RNA and protein homeostasis. Neuron, 79, 416-438.
Pollari, E., Goldsteins, G., Bart, G., Koistinaho, J. and Giniatullin, R. (2014) The role of oxidative stress in degeneration of the neuromuscular junction in amyotrophic lateral sclerosis. Frontiers in cellular neuroscience, 8, 131.
Stoica, R., De Vos, K. J., Paillusson, S., Mueller, S., Sancho, R. M., Lau, K. F., Vizcay-Barrena, G., Lin, W. L., Xu, Y. F., Lewis, J. et al. (2014) ER-mitochondria associations are regulated by the VAPB-PTPIP51 interaction and are disrupted by ALS/FTD-associated TDP-43. Nature communications, 5, 3996.
Tadic, V., Prell, T., Lautenschlaeger, J. and Grosskreutz, J. (2014) The ER mitochondria calcium cycle and ER stress response as therapeutic targets in amyotrophic lateral sclerosis. Frontiers in cellular neuroscience, 8, 147.
Prell, T., Lautenschlager, J., Weidemann, L., Ruhmer, J., Witte, O. W. and Grosskreutz, J. (2014) Endoplasmic reticulum stress is accompanied by activation of NF-kappaB in amyotrophic lateral sclerosis. Journal of neuroimmunology, 270, 29-36.
Ari, C., Poff, A. M., Held, H. E., Landon, C. S., Goldhagen, C. R., Mavromates, N. and D'Agostino, D. P. (2014) Metabolic Therapy with Deanna Protocol Supplementation Delays Disease Progression and Extends Survival in Amyotrophic Lateral Sclerosis (ALS) Mouse Model. PLoS One, 9, e103526.
Lekoubou, A., Matsha, T. E., Sobngwi, E. and Kengne, A. P. (2014) Effects of diabetes mellitus on amyotrophic lateral sclerosis: a systematic review. BMC research notes, 7, 171.
Lim, M. A., Bence, K. K., Sandesara, I., Andreux, P., Auwerx, J., Ishibashi, J., Seale, P. and Kalb, R. G. (2014) Genetically altering organismal metabolism by leptin-deficiency benefits a mouse model of Amyotrophic Lateral Sclerosis. Hum Mol Genet.
Wills, A. M., Hubbard, J., Macklin, E. A., Glass, J., Tandan, R., Simpson, E. P., Brooks, B., Gelinas, D., Mitsumoto, H., Mozaffar, T. et al. (2014) Hypercaloric enteral nutrition in patients with amyotrophic lateral sclerosis: a randomised, double-blind, placebo-controlled phase 2 trial. Lancet, 383, 2065-2072.
Lawton, K. A., Cudkowicz, M. E., Brown, M. V., Alexander, D., Caffrey, R., Wulff, J. E., Bowser, R., Lawson, R., Jaffa, M., Milburn, M. V. et al. (2012) Biochemical alterations associated with ALS. Amyotrophic lateral sclerosis: official publication of the World Federation of Neurology Research Group on Motor Neuron Diseases, 13, 110-118.
Bouteloup, C., Desport, J. C., Clavelou, P., Guy, N., Derumeaux-Burel, H., Ferrier, A. and Couratier, P. (2009) Hypermetabolism in ALS patients: an early and persistent phenomenon. J Neurol, 256, 1236-1242.
Dupuis, L., Oudart, H., Rene, F., Gonzalez de Aguilar, J. L. and Loeffler, J. P. (2004) Evidence for defective energy homeostasis in amyotrophic lateral sclerosis: benefit of a high-energy diet in a transgenic mouse model. Proc Natl Acad Sci USA, 101, 11159-11164.
Jawaid, A., Salamone, A. R., Strutt, A. M., Murthy, S. B., Wheaton, M., McDowell, E. J., Simpson, E., Appel, S. H., York, M. K. and Schulz, P. E. (2010) ALS disease onset may occur later in patients with pre-morbid diabetes mellitus. European journal of neurology: the official journal of the European Federation of Neurological Societies, 17, 733-739.
Estes, P. S., Boehringer, A., Zwick, R., Tang, J. E., Grigsby, B. and Zarnescu, D. C. (2011) Wild-type and A315T mutant TDP-43 exert differential neurotoxicity in a Drosophila model of ALS. Human molecular genetics, 20, 2308-2321.
Li, Y., Ray, P., Rao, E. J., Shi, C., Guo, W., Chen, X., Woodruff, E. A., 3rd,
Fushimi, K. and Wu, J. Y. (2010) A Drosophila model for TDP-43 proteinopathy. Proc Natl Acad Sci USA, 107, 3169-3174.
Estes, P. S., Daniel, S. G., McCallum, A. P., Boehringer, A. V., Sukhina, A. S., Zwick, R. A. and Zarnescu, D. C. (2013) Motor neurons and glia exhibit specific individualized responses to TDP-43 expression in a Drosophila model of amyotrophic lateral sclerosis. Disease models & mechanisms, 6, 721-733.
Jia, Z., Sun, Y., Yang, G., Zhang, A., Huang, S., Heiney, K. M. and Zhang, Y. (2014) New Insights into the PPAR gamma Agonists for the Treatment of Diabetic Nephropathy. PPAR research, 2014, 818530.
Staels, B. and Fruchart, J. C. (2005) Therapeutic roles of peroxisome proliferator-activated receptor agonists. Diabetes, 54, 2460-2470.
Forman, B. M., Chen, J. and Evans, R. M. (1996) The peroxisome proliferator-activated receptors: ligands and activators. Ann N Y Acad Sci, 804, 266-275.
Kapadia, R., Yi, J. H. and Vemuganti, R. (2008) Mechanisms of anti-inflammatory and neuroprotective actions of PPAR-gamma agonists. Front Biosci, 13, 1813-1826.
Rutherford, N. J., Zhang, Y. J., Baker, M., Gass, J. M., Finch, N. A., Xu, Y. F., Stewart, H., Kelley, B. J., Kuntz, K., Crook, R. J. et al. (2008) Novel mutations in TARDBP (TDP-43) in patients with familial amyotrophic lateral sclerosis. PLoS Genet, 4, e1000193.
Gustafson, K. and Boulianne, G. L. (1996) Distinct expression patterns detected within individual tissues by the GAL4 enhancer trap technique. Genome, 39, 174-182.
Dupuis, L., Dengler, R., Heneka, M. T., Meyer, T., Zierz, S., Kassubek, J., Fischer, W., Steiner, F., Lindauer, E., Otto, M. et al. (2012) A randomized, double blind, placebo-controlled trial of pioglitazone in combination with riluzole in amyotrophic lateral sclerosis. PLoS One, 7, e37885.
Boyer, J. G., Ferrier, A. and Kothary, R. (2013) More than a bystander: the contributions of intrinsic skeletal muscle defects in motor neuron diseases. Frontiers in physiology, 4, 356.
Philips, T. and Rothstein, J. D. (2014) Glial cells in amyotrophic lateral sclerosis. Exp Neurol.
Budnik, V., Koh, Y. H., Guan, B., Hartmann, B., Hough, C., Woods, D. and Gorczyca, M. (1996) Regulation of synapse structure and function by the Drosophila tumor suppressor gene dlg. Neuron, 17, 627-640.
Lanson, N. A., Jr., Maltare, A., King, H., Smith, R., Kim, J. H., Taylor, J. P., Lloyd, T. E. and Pandey, U. B. (2011) A Drosophila model of FUS-related neurodegeneration reveals genetic interaction between FUS and TDP-43. Human molecular genetics.
Wang, J. W., Brent, J. R., Tomlinson, A., Shneider, N. A. and McCabe, B. D. (2011) The ALS-associated proteins FUS and TDP-43 function together to affect Drosophila locomotion and life span. The Journal of clinical investigation, 121, 4118-4126.
Kiaei, M., Kipiani, K., Chen, J., Calingasan, N. Y. and Beal, M. F. (2005) Peroxisome proliferator-activated receptor-gamma agonist extends survival in transgenic mouse model of amyotrophic lateral sclerosis. Exp Neurol, 191, 331-336.
Schutz, B., Reimann, J., Dumitrescu-Ozimek, L., Kappes-Horn, K., Landreth, G. E., Schurmann, B., Zimmer, A. and Heneka, M. T. (2005) The oral antidiabetic pioglitazone protects from neurodegeneration and amyotrophic lateral sclerosis-like symptoms in superoxide dismutase-G93A transgenic mice. J Neurosci, 25, 7805-7812.
Watson, M. R., Lagow, R. D., Xu, K., Zhang, B. and Bonini, N. M. (2008) A drosophila model for amyotrophic lateral sclerosis reveals motor neuron damage by human SOD1. J Biol Chem, 283, 24972-24981.
Bruijn, L. I., Becher, M. W., Lee, M. K., Anderson, K. L., Jenkins, N. A., Copeland, N. G., Sisodia, S. S., Rothstein, J. D., Borchelt, D. R., Price, D. L. et al. (1997) ALS-linked SOD1 mutant G85R mediates damage to astrocytes and promotes rapidly progressive disease with SOD1-containing inclusions. Neuron, 18, 327-338.
Gurney, M. E., Pu, H., Chiu, A. Y., Dal Canto, M. C., Polchow, C. Y., Alexander, D. D., Caliendo, J., Hentati, A., Kwon, Y. W., Deng, H. X. et al. (1994) Motor neuron degeneration in mice that express a human Cu, Zn superoxide dismutase mutation. Science, 264, 1772-1775.
Gillies, P. S. and Dunn, C. J. (2000) Pioglitazone. Drugs, 60, 333-343; discussion 344-335.
Gray, E., Ginty, M., Kemp, K., Scolding, N. and Wilkins, A. (2012) The PPAR-gamma agonist pioglitazone protects cortical neurons from inflammatory mediators via improvement in peroxisomal function. Journal of neuroinflammation, 9, 63.
Colca, J. R., McDonald, W. G., Waldon, D. J., Leone, J. W., Lull, J. M., Bannow, C. A., Lund, E. T. and Mathews, W. R. (2004) Identification of a novel mitochondrial protein (“mitoNEET”) cross-linked specifically by a thiazolidinedione photoprobe. Am J Physiol Endocrinol Metab, 286, E252-260.
Garcia-Ruiz, I., Solis-Munoz, P., Fernandez-Moreira, D., Munoz-Yague, T. and Solis-Herruzo, J. A. (2013) Pioglitazone leads to an inactivation and disassembly of complex I of the mitochondrial respiratory chain. BMC Biol, 11, 88.
King-Jones, K. and Thummel, C. S. (2005) Nuclear receptors—a perspective from Drosophila. Nature reviews. Genetics, 6, 311-323.
Rothstein, J. D. (2009) Current hypotheses for the underlying biology of amyotrophic lateral sclerosis. Ann Neurol, 65 Suppl 1, S3-9.
Blokhuis, A. M., Groen, E. J., Koppers, M., van den Berg, L. H. and Pasterkamp, R. J. (2013) Protein aggregation in amyotrophic lateral sclerosis. Acta Neuropathol, 125, 777-794.
Zhao, Z., Lange, D. J., Voustianiouk, A., MacGrogan, D., Ho, L., Suh, J., Humala, N., Thiyagarajan, M., Wang, J. and Pasinetti, G. M. (2006) A ketogenic diet as a potential novel therapeutic intervention in amyotrophic lateral sclerosis. BMC neuroscience, 7, 29.
Renton, A. E., Chio, A. and Traynor, B. J. (2014) State of play in amyotrophic lateral sclerosis genetics. Nat Neurosci, 17, 17-23.
Benatar, M. (2007) Lost in translation: treatment trials in the SOD1 mouse and in human ALS. Neurobiol Dis, 26, 1-13.
Scott, S., Kranz, J. E., Cole, J., Lincecum, J. M., Thompson, K., Kelly, N., Bostrom, A., Theodoss, J., Al-Nakhala, B. M., Vieira, F. G. et al. (2008) Design, power, and interpretation of studies in the standard murine model of ALS. Amyotroph Lateral Scler, 9, 4-15.
Kabashi, E., Bercier, V., Lissouba, A., Liao, M., Brustein, E., Rouleau, G. A. and Drapeau, P. (2011) FUS and TARDBP but not SOD1 interact in genetic models of amyotrophic lateral sclerosis. PLoS genetics, 7, e1002214.
Levine, T. D., Bowser, R., Hank, N. C., Gately, S., Stephan, D., Saperstein, D. S. and Van Keuren-Jensen, K. (2012) A Pilot Trial of Pioglitazone HCl and Tretinoin in ALS: Cerebrospinal Fluid Biomarkers to Monitor Drug Efficacy and Predict Rate of Disease Progression. Neurology research international, 2012, 582075.
Karak, M., Bal, N. C., Bal, C. and Sharon, A. (2013) Targeting peroxisome proliferator-activated receptor gamma for generation of antidiabetic drug. Curr Diabetes Rev, 9, 275-285.
Jawaid, A., Murthy, S. B., Wilson, A. M., Qureshi, S. U., Amro, M. J., Wheaton, M., Simpson, E., Harati, Y., Strutt, A. M., York, M. K. et al. (2010) A decrease in body mass index is associated with faster progression of motor symptoms and shorter survival in ALS. Amyotroph Lateral Scler, 11, 542-548.
Brand, A. H. and Perrimon, N. (1993) Targeted gene expression as a means of altering cell fates and generating dominant phenotypes. Development, 118, 401-415.
Storey, J. D. and Tibshirani, R. (2003) Statistical significance for genomewide studies. Proc Natl Acad Sci USA, 100, 9440-9445.

### 12. Example 6

Recently, alterations in cellular metabolism have taken center-stage in the field of Amyotrophic Lateral Sclerosis (ALS). Several studies indicate that diabetic patients have a later onset of ALS, and that mild to moderate obesity is a potential protective factor. Compounded with these observations are the findings that ALS patients exhibit hypermetabolism. Consistently, some reports indicate that a high-calorie diet in the form of high carbohydrate and/or high fat is safe, well-tolerated and mildly protective in patients. Thus, glucose and fatty acid metabolic pathways are emerging as attractive new approaches for therapeutic development. Using a Drosophila model of ALS based on human TDP-43, a protein involved in ALS and related neurodegenerative diseases, a global metabolomic study has been performed. The results indicate alterations in several key biosynthetic pathways, including, but not limited to, neurotransmitter synthesis and degradation, glucose uptake and oxidation as well as lipid beta oxidation. The latter indicates mitochondrial dysfunction, which has previously been implicated in ALS. Interestingly, cellular hypermetabolism in the form of elevated TCA cycle, which is now considered a hallmark of the disease in patients, is also observed in this fly model. Given other findings in this Drosophila model, the TCA cycle upregulation is likely a compensatory response to alterations in glucose and lipid metabolism. These findings not only reinforce the validity of this model system but also pinpoint possible therapeutic interventions based on nutritional supplementation and genetic intervention. This approach is unique in several ways: 1) it gives an in vivo metabolic signature that can provide “biomarkers” for the majority of ALS cases with TDP-43 pathology, 2) it provides TDP-43 variant specific metabolic signatures that can be used to formulate personalized therapeutics, and 3) it provides valuable information about pathways altered in disease states, which in turn, can be used for therapeutic intervention. Furthermore, given the unavailability of large cohorts of patient data, this approach is unparalleled as far as ease and cost are concerned. ALS flies were treated with pioglitazone, a drug effective in mitigating several aspects of the disease, and metabolomic profiling was performed. The data show that some of the TDP-induced changes in metabolites are indeed restored to control levels by the drug. Thus we propose that this global metabolomic approach is a useful and effective way of dissecting disease mechanisms, and will inform future therapeutic approaches.

What is claimed is:

1. An in vivo method of screening for a therapeutic for amyotrophic lateral sclerosis, the method comprising:
administering a candidate therapeutic for amyotrophic lateral sclerosis to Drosophila larvae, wherein the larvae express a human TDP-43 transgene; and
determining the survival of the larvae to an adult stage;
wherein the survival of the larvae indicates that the candidate therapeutic for amyotrophic lateral sclerosis is a therapeutic for amyotrophic lateral sclerosis.

2. The method of claim 1, wherein the human TDP-43 transgene is expressed in motor neurons.

3. The method of claim 1, wherein the human TDP-43 transgene comprises one or more mutations.

4. The method of claim 3, wherein the one or more mutations correspond to a D169G, G298S, A315T, or N345K amino acid substitution.

5. The method of claim 3, wherein the human TDP-43 transgene is expressed in motor neurons.

6. The method of claim 1, wherein the therapeutic for amyotrophic lateral sclerosis ameliorates one or more signs or symptoms associated with amyotrophic lateral sclerosis.

7. The method of claim 6 wherein the one or more signs or symptoms associated with amyotrophic lateral sclerosis comprise phenotypic signs or symptoms.

8. The method of claim 1, further comprising examining neuromuscular junctions, characterizing locomotion, quantifying motor neuron cell death, examining eye neuroepithelium, identifying cytoplasmic inclusions, or a combination thereof.

9. A transgenic Drosophila comprising a human TDP-43 gene.

10. The transgenic Drosophila of claim 9, wherein the TDP-43 gene comprises one or more mutations.

11. A therapeutic for amyotrophic lateral sclerosis identified by the method of claim 1.
